(19) United States
(12) Patent Application Publication
(10) Pub. No.: US 2012/0269826 A1
(43) Pub. Date: Oct. 25, 2012

(54) ANTI-CD3 ANTIBODY DOSING IN AUTOIMMUNE DISEASE

(76) Inventors: Charlotte McKee, Newton, MA (US); Paul Ponath, San Francisco, CA (US); Douglas Ringler, Boston, MA (US); Michael Rosenzweig, Boston, MA (US); Lou Vaickus, Hingham, MA (US)

(21) Appl. No.: 13/502,618

(22) PCT Filed: Oct. 20, 2010
(86) PCT No.: PCT/US10/53438

§ 371 Date: Jul. 9, 2012

Related U.S. Application Data
(60) Provisional application No. 61/253,482, filed on Oct. 20, 2009.

(51) Int. Cl. A61K 39/395; A61P 37/06

(52) U.S. Cl. 424/172.1

(57) ABSTRACT

Provided herein are methods of administering anti-CD3 antibodies or antigen-binding fragments thereof to an animal. In certain embodiments, the anti-CD3 antibody or fragment thereof does not bind or has reduced binding to at least one class of Fc (gamma) receptors. In certain embodiments, the animal has an immune-related disease.

### RELATED APPLICATIONS

The present application claims the benefit of U.S. Provisional Patent Application Ser. No. 61/253,482, filed Oct. 20, 2009, which is herein incorporated by reference in its entirety.

### TECHNICAL FIELD

Provided herein are methods of administering anti-CD3 antibodies or antigen binding fragments thereof to an animal.

### BACKGROUND

CD3 is part of a functional T cell receptor (TCR) complex found on the surface membranes of T lymphocytes. This complex is referred to interchangeably herein as the CD3/TCR complex or the CD3/TCR complex. In mammals, CD3 is a protein complex composed of several distinct polypeptide chains: a CD3-gamma chain, a CD3-delta chain, two CD3-epsilon chains, and two CD3-zeta chains. These chains associate with either an alpha/beta or a gamma/delta TCR complex to generate a functional CD3/TCR complex. Binding of a CD3/TCR complex to a peptide antigen presented on a MHC molecule leads to transduction of a signal (e.g., an activating signal, a suppressive signal, or an inactivating signal) from the CD3/TCR complex to the metabolic machinery of the relevant T cell.

Antibodies against the CD3 molecule have been tested for efficacy in the treatment of certain immune-related diseases in humans such as diabetes and psoriasis. Cytokine release syndrome and other negative effects are persistent problems in antibody-based therapeutic approaches, including therapeutic approaches involving anti-CD3 antibodies. Methods of administering anti-CD3 antibodies that overcome such problems would be advantageous.

### SUMMARY

Provided herein are methods of administering anti-CD3 antibodies or antigen binding fragments thereof to an animal. In certain embodiments, methods disclosed herein permit administration of higher cumulative doses of the anti-CD3 antibody or fragment with decreased pro-inflammatory cytokine release and immunogenicity, and no perturbation (eliminate or decrease) of Epstein Barr Virus immunity. In certain embodiments, methods disclosed herein facilitate higher individual doses of anti-CD3 antibodies or fragments later in a dosing regimen than would be possible with traditional dosing regimens.

In one embodiment, the present document provides a method of treating a human with an anti-CD3 antibody or an antigen binding fragment thereof. The method can include: administering the antibody or the fragment to the human in a regimen such that: (a) in a therapy window of at least two days and no more than 6 days, for at least 48 hours (e.g., at least: 50 hours, 52 hours, 54 hours, 56 hours, 58 hours, 60 hours, 65 hours, 70 hours, 75 hours, 80 hours, 90 hours, 100 hours, 110 hours, 120 hours, 130 hours, 140 hours; or 144 hours) of the window, the mean level of free CD3/TCR complexes on CD4+ and on CD8+ T cells can be at least 10 percent and less than 40 percent of the mean baseline level; or (b) in a therapy window of 7 days or more, for at least 48 hours (e.g., 50 hours, 52 hours, 54 hours, 56 hours, 58 hours, 60 hours, 65 hours, 70 hours, 75 hours, 80 hours, 90 hours, 100 hours, 110 hours, 120 hours, 130 hours, 140 hours, or 144 hours) of the first 6 days of the window, the mean level of free CD3/TCR complexes on CD4+ and on CD8+ T cells can be at least 10 percent and less than 40 percent of the mean baseline level; or (c) in a therapy window of at least 8 days, for at least 48 hours (e.g., at least: 50 hours, 52 hours, 54 hours, 56 hours, 58 hours, 60 hours, 65 hours, 70 hours, 75 hours, 80 hours, 90 hours, 100 hours, 110 hours, 120 hours, 130 hours, 140 hours, 150 hours, 160 hours, 170 hours, 180 hours, 190 hours; or 192 hours) of the window, the mean level of free CD3/TCR complexes on CD4+ and on CD8+ T cells can be at least 10 percent and less than 40 percent of the mean baseline level and at least 30 (e.g., at least: 32 hours, 34 hours, 36 hours, 38 hours, 40 hours; 44 hours; or 48 hours) of the 48 hours occur after the first 6 days of the window; or (d) in a therapy window of at least 4 days, for at least 90 hours (e.g., at least: 92 hours, 94 hours; or 96 hours) of the window, the mean level of free CD3/TCR complexes on CD4+ and on CD8+ T cells can be at least 10 percent and less than 40 percent of the mean baseline level. In the method, the antibody or fragment does not bind, or has reduced binding, to at least one class of Fc (gamma) receptor as compared to the OKT3 antibody. Moreover, for the above alternatives (b), (c), and (d) above, the regimen, the therapy window, or both the regimen and the therapy window can be 14 days or more. On the other hand, for the above alternatives (b), (c) and (d) above, the regimen, the therapy window, or both the regimen and the therapy window may not be more than 14 days. The time of the therapy window in which the mean level of free CD3/TCR complexes is at least 10 percent and less than 40 percent of the mean baseline level can be continuous or not continuous. Furthermore, in the method, at least one dose of the antibody or fragment administered in an administration can be greater than 0.5 mg (e.g., greater than: 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, 1.5 mg, 1.55 mg, 1.6 mg, 1.65 mg, 1.7 mg, 1.75 mg 1.8 mg, 1.85 mg, 1.9 mg, 1.95 mg, 2.0 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, or 2.95 mg) and the maximum daily dose can be no greater than 3.0 mg (e.g., no greater than: 0.55, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, 1.5 mg, 1.55 mg, 1.6 mg, 1.65 mg, 1.7 mg, 1.75 mg 1.8 mg, 1.85 mg, 1.9 mg, 1.95 mg, 2.0 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, or 2.95 mg). In addition, the maximum daily dose of the antibody or the fragment is 1.75 mg or less (e.g., 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, 1.5 mg, 1.55 mg, 1.6 mg, 1.65 mg, 1.7 mg, or less).

In a further embodiment, the present document features an additional method of treating a human with an anti-CD3 antibody or an antigen binding fragment thereof. The method can include: administering the antibody or the fragment to the human in a regimen such that: (a) in a therapy window of at least two days and no more than 6 days, for at least 12 hours (e.g., at least: 14 hours, 16 hours, 18 hours, 20 hours, 22 hours, 24 hours, 26 hours, 28 hours, 30 hours, 35 hours, 40 hours, 45 hours, 50 hours, 55 hours, 60 hours, 65 hours, 70 hours, 75 hours, 80 hours, 90 hours, 100 hours, 110 hours, 120 hours, 130 hours, 140 hours, or 144 hours) of the window, the mean level of free CD3/TCR complexes on CD4+ and on CD8+ T cells can be at least 20 percent and less than 30 percent of the mean baseline level; or (b) in a therapy window of 7 days or more, for at least 18 hours (e.g., at least: 20 hours, 22 hours, 24 hours, 26 hours, 28 hours, 30 hours, 35 hours, 40 hours, 45 hours, 50 hours, 55 hours, 60 hours, 65 hours, 70 hours, 75 hours, 80 hours, 90 hours, 100 hours, 110 hours, 120 hours, 130 hours, 140 hours, or 144 hours) of the first 6 days of the window, the mean level of free CD3/TCR complexes on CD4+ and on CD8+ T cells can be at least 20 percent and less than 30 percent of the mean baseline level; (c) in a therapy window of at least 7 days, for at least 24 hours (e.g., at least: 26 hours, 28 hours, 30 hours, 32 hours, 34 hours, 36 hours, 38 hours, 40 hours, 42 hours, 44 hours, 46 hours, 48 hours, 50 hours, 55 hours, 60 hours, 65 hours, 70 hours, 75 hours, 80 hours, 90 hours, 100 hours, 110 hours, 120 hours, 130 hours, 140 hours, 150 hours, 160 hours, or 168 hours) of the window, the mean level of free CD3/TCR complexes on CD4+ and on CD8+ T cells can be at least 20 percent and less than 30 percent of the mean baseline level and at least 15 of the at least 30 hours occur after the first 6 days of the window; or (d) in a therapy window of at least 7 days, for at least 40 hours (e.g., at least: 42 hours, 44 hours, 46 hours, 48 hours, 50 hours, 52 hours, 54 hours, 58 hours, 60 hours, 65 hours, 70 hours, 75 hours, 80 hours, 90 hours, 100 hours, 110 hours, 120 hours, 130 hours, 140 hours, 150 hours, 160 hours, or 168 hours) of the window, the mean level of free CD3/TCR complexes on CD4+ and on CD8+ T cells can be at least 20 percent and less than 30 percent of the mean baseline level and at least half (at least: 55%, 60%, 65%, 70%, 75%, 80%, 85%, 90%, 95%, 98%; or 100%) of the at least 40 hours in the window occur after the first 6 days of the window. In the method, the antibody or fragment does not bind, or has reduced binding, to at least one class of Fc (gamma) receptor as compared to the OKT3 antibody. Moreover, for the above alternatives (b), (c), and (d) above, the regimen, the therapy window, or both the regimen and the therapy window can be 14 days or more. On the other hand, for the above alternatives (b), (c) and (d) above, the regimen, the therapy window, or both the regimen and the therapy window may not be more than 14 days. Moreover, in the method, the time within the therapy window in which the mean level of free CD3/TCR complexes at least 20 percent and less than 30 percent of the mean baseline level can be not continuous. In addition, the first at least four days of the regimen can be a dosing ramp.

In an additional embodiment, the present document provides another method of treating a human with an anti-CD3 antibody or an antigen binding fragment thereof. The method can involve: administering the antibody or the fragment to the human in a regimen such that: (a) in a regimen of 3 days or more, the dose administered on each of at least 3 days of the regimen is at least 1 mg (e.g., at least: 1.5 mg, 2.0 mg, or 2.5 mg) and no greater than 3 mg; (b) in a regimen of 3 days or more, the daily dose administered is at least 1 mg (e.g., at least: 1.1 mg, 1.15 mg, 1.2 mg, 1.25 mg, 1.5 mg, 1.55 mg, 1.6 mg, 1.65 mg, 1.7 mg, or 1.75 mg) and no greater than 1.75 mg in any 24 hour period and on each of at least 3 days of the regiment; (c) in a regimen of 3 days or more, the daily dose administered is at least 14 μg/kg (e.g., at least: 18 μg/kg, 22 μg/kg, 28 μg/kg, 34 μg/kg, 40 μg/kg, or 42 μg/kg) and no greater than 42 μg/kg in any 24 hour period and on each of at least 3 days of the regimen; (d) in a regimen of 3 days or more, the total dose administered is 2.5 mg (e.g., 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, or 9.0 mg) to 9.0 mg and no greater than 3 mg on any single day of the regimen; (e) in a regimen of 3 days or more, the total dose administered is 2.5 mg (e.g., 3.0 mg, 3.5 mg, 4.0 mg, 4.5 mg, 5.0 mg, 5.5 mg, 6.0 mg, 6.5 mg, or 6.6 mg) to 6.6 mg and no greater than 2.2 mg on any single day of the regimen; (f) in a regimen 3 days or more, the total dose administered is 35 μg/kg (e.g., 45 μg/kg, 55 μg/kg, 65 μg/kg, 75 μg/kg, 85 μg/kg, or 93 μg/kg) to 93 μg/kg and no greater than 31 μg/kg on any single day of the regimen; (g) in a regimen 3 days or more, the total dose administered is 35 μg/kg (e.g., 45 μg/kg, 55 μg/kg, 65 μg/kg, 75 μg/kg, 85 μg/kg, 95 μg/kg, 105 μg/kg, 115 μg/kg or 126 μg/kg) to 126 μg/kg and no greater than 42 μg/kg on any single day of the regimen; (h) in a therapy window of at least three days, where a dose is administered over a period of 24 hours or more, the total dose administered to the human is at least 2.5 mg (e.g., at least: 2.6 mg, 2.7 mg, 2.8 mg, 2.9 mg, 3.0 mg, 3.1 mg, 3.2 mg, 3.3 mg, 3.4 mg, or 3.5 mg); or (i) in a therapy window of at least three days, where a dose is administered over a period of 24 hours or more, the total dose administered to the human is at least 35 μg/kg (e.g., 45 μg/kg, 55 μg/kg, 65 μg/kg, 75 μg/kg, 85 μg/kg, 95 μg/kg, or 100 μg/kg). In the method, the antibody or fragment does not bind, or has reduced binding, to at least one class of Fc (gamma) receptor as compared to the OKT3 antibody and, optionally, the three days are not continuous.

In yet another embodiment, the present document provides a method of treating a human with an anti-CD3 antibody, or an antigen binding fragment thereof. The method can include administering the antibody or fragment to the human in a regimen that comprises a dosing ramp of at least four (e.g., at least: four, five, six, seven, eight, nine, or ten) days. In the method, the antibody or fragment does not bind or has reduced binding to at least one class of the Fc (gamma) receptor as compared to the OKT3 antibody. Moreover, in the method, for at least days two to four of the ramp, the dosing can produce a daily decrease in the mean maximum levels of free CD3/TCR complexes on CD4+ and on CD8+ T cells as compared to the mean baseline levels, wherein the differences between the mean maximum levels on any day of the at least day two to day four of the ramp and the mean maximum levels on the preceding day are not greater than 25 percent (e.g., not greater than: 20 percent, 15 percent, 10 percent, or 5 percent) of the mean maximum levels on the preceding day. The first dose of the ramp can produce a decrease in the mean maximum levels of free CD3/TCR complexes on CD4+ and on CD8+ T cells as compared to the mean baseline levels of no greater than 30 percent of the mean maximum levels preceding the first dose of the ramp. In addition, for at least days two to four of the ramp, the dosing can produce a daily decrease in mean maximum levels of free TCR complex molecules on CD4+ and on CD8+ T cells as compared to the mean baseline levels, wherein the differences between the mean maximum levels on any day of the at least day two to day four of the ramp and the mean maximum levels on the preceding day are at least 5 percent of the mean maximum levels on the preceding day. Moreover, for at least days two through four of the ramp, the dosing of the ramp can produce a daily increase in minimum concentration of the anti-CD3 antibody or the fragment (Cₘᵢₙ) in the peripheral blood, optionally peripheral blood plasma, of the human. Also, for at least days two through four of the ramp, the dosing can produce a daily increase in the Cₘᵢₙ in the peripheral blood or peripheral blood plasma of the human of no greater than 2.5 times (e.g., no greater than: 2.0 times, 1.5 time, or 1.0 times) the Cₘᵢₙ in the peripheral blood or peripheral blood plasma on the preceding day, when the concentration of the antibody or fragment in the peripheral blood or peripheral blood plasma of the human is greater than 0.002 mg/L (e.g., greater than: 0.004 mg/L, 0.006 mg/ml, 0.008 mg/ml, 0010 mg/ml, or 0.012 mg/ml). The first dose of the ramp produces a Cₘᵢₙ in the peripheral blood or peripheral blood plasma of the human of no greater than 0.01 mg/L. Furthermore, for at least days two through four of the ramp, the dosing produces a daily increase in Cₘᵢₙ in the peripheral blood or peripheral blood plasma of the human of at least 10 percent (e.g., at least: 12%, 14%, 16%, 18%, 20%, 25%, 30%, or 40%) as compared to the Cₘᵢₙ in the peripheral blood or peripheral blood plasma of the human on the preceding day.

The following embodiments apply to all the described methods and their embodiments. Thus, in such methods, the anti-CD3 antibody or antigen binding fragment thereof can be administered in a dosing regimen of at least five days; the antibody or fragment can be administered on day one; the amount of antibody or fragment administered on each of days one and two does not exceed 0.5 mg per day; the amount of antibody or fragment administered on day three can be less than about 0.5 mg greater than the amount of antibody or fragment administered on day two; the amount of antibody or fragment administered on day four can be less than about 0.55 mg greater than the amount of antibody or fragment administered on day three; the amount of antibody or fragment administered on day five can be less than about 0.6 mg greater than the amount of antibody or fragment administered on day four; the amount of antibody or fragment administered on day five can be more than 0.3 mg greater than the amount of antibody or fragment administered on day two; and the amount of antibody or fragment administered on day five is at least about 0.5 mg. In any of the methods one or more pre-ramp doses are administered prior to dose day one. In any of the above methods, the ramp can be given prior to the administration of a maximum daily dose and causes a reduction in one or both of the (a) production of at least one pro-inflammatory cytokine or tryptase and (b) immunogenicity, as compared to one or both of the (i) production of the at least one pro-inflammatory cytokine or tryptase and (ii) immunogenicity, respectively, that is observed after administration of the maximum dose without a ramp of at least four days. The at least one pro-inflammatory cytokine can be IL2, IL6, IL10, IFN-gamma, or TNF-alpha. In addition, in the above methods, the antibody or fragment can be administered in the following dosing regimen: the amount of antibody or fragment administered on day one is about 0.1 mg; the amount of antibody or fragment administered on day two is about 0.2 mg; the amount of antibody or fragment administered on day three is about 0.3 mg; the amount of antibody or fragment administered on day four is about 0.75 mg; the amount of antibody or fragment administered on day five is about 1.0 mg; the amount of antibody or fragment administered on day six is about 1.25 mg; the amount of antibody or fragment administered on day seven is about 1.5 mg; and the amount of antibody or fragment administered on day eight is about 1.75 mg. Alternatively, in the above methods, the antibody or fragment can be administered in the following dosing regimen: the amount of antibody or fragment administered on day one is about 0.2 mg; the amount of antibody or fragment administered on day two is about 0.4 mg; the amount of antibody or fragment administered on day three is about 0.6 mg; the amount of antibody or fragment administered on day four is about 0.8 mg; and the amount of antibody or fragment administered on day five is about 1.1 mg. Furthermore, in any of the above methods, the method further comprises administration of one or more additional agents selected from the group consisting of analgesics, anti-histamines, anti-inflammatories, anti-emetics, and therapeutic agents. Any of the method further can further include one or more additional regimens comprising administration of the anti-CD3 antibody or an antigen binding fragment or a different anti-CD3 antibody or antigen binding fragment thereof. In addition, in any of the methods, the antibody or fragment can have a binding affinity constant of at least 0.968 μg/mL and a kel of about 1.39 day-1; moreover the antibody or fragment can have an IC50 of less than 75 ng/ml. Moreover, in these methods, the antibody can have a half-life of between 5 and 20 hours at the doses administered in the regimen. The antibody used in any of the methods can be an aglycosylated monoclonal antibody comprising a humanized γ heavy chain and a rat/human chimeric λ light chain. Furthermore, the methods can cause modulation in the activity or numbers of one or both of antigen-specific effector (Teff) or antigen-specific regulatory (Treg) T cells, e.g., the number of antigen-specific T regulatory cells can be enhanced. In addition, in the methods, on at least one day of the treatment window, the mean levels of CD3/TCR complexes on CD4+ and on CD8+ T-cells are decreased by at least 20% (e.g., at least: 25%, 30%, 35%, 40%, 45%, 50%, 55%, 60%, 65%, 70%, 75%, 80%, or 85%) and less than 90% as compared to the mean baseline levels. The human that is treated with any of the above methods can have an immune-related disease, e.g., type I diabetes, type II diabetes, psoriasis, rheumatoid arthritis, lupus, inflammatory bowel disease, ulcerative colitis, Crohn's disease, Graves thyroiditis, Graves ophthalmopathy, Metabolic Syndrome, multiple sclerosis, a pathological condition resulting from organ or tissue transplantation, graft versus host disease, or myasthenia gravis.

In certain embodiments, methods disclosed herein comprise administering an anti-CD3 antibody or antigen binding fragment thereof, both of which do not bind or have reduced binding to at least one class of Fc (gamma) receptor compared to the OKT3 antibody, e.g., at least 50% reduced binding. In certain embodiments, methods disclosed herein comprise administering an anti-CD3 antibody or fragment, both of which do not bind or have reduced binding to at least one class of Fc (gamma) receptor compared to the IgG1 antibody produced by the ARH-77 cell line deposited under ATCC catalog number CRL-1621, e.g., at least 50% reduced binding. In certain embodiments, the anti-CD3 antibody or fragment is administered over a dosing regimen of at least five days or at least eight days.

In certain embodiments, the anti-CD3 antibody or antigen binding fragment thereof is administered on day one of the dosing regimen, and the amount of anti-CD3 antibody or fragment administered on each of days one and two does not exceed 0.5 mg per day, e.g., does not exceed 0.2 mg per day or 0.3 mg per day. In certain embodiments, the amount of the anti-CD3 antibody or fragment administered on day one is about 0.1 mg, about 0.2 mg, or about 0.3 mg.

In certain embodiments, the amount of anti-CD3 antibody or antigen binding fragment thereof administered on day three of the dosing regimen is less than about 0.5 mg greater than the amount of the anti-CD3 antibody or fragment administered on day two, e.g., about 0.1 mg greater or about 0.2 mg greater. In certain embodiments, the amount of anti-CD3 antibody or fragment the administered on day four is less than about 0.55 mg greater than the amount of the anti-CD3 antibody or fragment administered on day three, e.g., about 0.4 mg greater or about 0.45 mg greater. In certain embodiments, the amount of the anti-CD3 antibody or fragment administered on day five is less than about 0.6 mg greater than the amount of the anti-CD3 antibody or fragment administered on day four, e.g., about 0.25 mg greater or about 0.4 mg greater. In certain embodiments, the amount of the anti-CD3 antibody or fragment administered on day five is more than 0.3 mg greater than the amount of anti-CD3 antibody or fragment thereof administered on day two, e.g., more than about 0.75 mg greater or more than about 1.0 mg greater. In certain embodiments, the amount of anti-CD3 antibody or fragment thereof administered on day five is at least about 0.5 mg.

In certain embodiments, the amount of anti-CD3 antibody or antigen binding antibody fragment thereof administered is about 0.1 mg on day one, about 0.2 mg on day two, about 0.3 mg on day three, and about 0.75 mg on each of days four through eight. In certain embodiments, the amount of anti-CD3 antibody or fragment thereof administered is about 0.1 mg on day one; about 0.2 mg on day two, about 0.3 mg on day 3, about 0.75 mg on day four, about 1.0 mg on day five, about 1.25 mg on day six, about 1.5 mg on day seven, and about 1.75 mg on day eight. In certain embodiments, the amount of anti-CD3 antibody or fragment thereof administered is about 0.1 mg on day one; about 0.2 mg on day two, about 0.3 mg on day 3, about 0.75 mg on day four, about 1.0 mg on day five, about 1.25 mg on day six, about 1.5 mg on day seven, and about 3.75 mg on day eight. In certain embodiments, the amount of the anti-CD3 antibody or fragment administered is about 0.2 mg on day one; about 0.4 mg on day two, about 0.6 mg on day 3, about 0.8 mg on day four, and about 1.1 mg on day five.

In certain embodiments, the anti-CD3 antibody or antigen binding fragment thereof is administered over a dosing regimen comprising at least four ramp days. In certain embodiments, the anti-CD3 antibody or fragment is administered in an amount greater than about 0.1 mg and less than about 0.5 mg on ramp day one. In certain embodiments, the amount of the anti-CD3 antibody or fragment administered on ramp day two is less than about 0.5 mg greater than the amount of the anti-CD3 antibody or fragment administered on ramp day one, e.g., about 0.1 mg greater or about 0.2 mg greater. In certain embodiments, the amount of the anti-CD3 antibody or fragment administered on ramp day three is less than about 0.55 mg greater than the amount of the anti-CD3 antibody or fragment administered on ramp day two, e.g., about 0.4 mg greater or about 0.45 mg greater. In certain embodiments, the amount of the anti-CD3 antibody or fragment administered on ramp day four is less than about 0.6 mg greater than the amount of the anti-CD3 antibody or fragment administered on ramp day three, e.g., about 0.25 mg greater or about 0.4 mg greater. In certain embodiments, the amount of the anti-CD3 antibody or fragment administered on ramp day four is more than 0.3 mg greater than the amount of the anti-CD3 antibody or fragment administered on ramp day one, e.g., more than about 0.75 mg greater or more than about 1.0 mg greater. In certain embodiments, the amount of the anti-CD3 antibody or fragment administered at least one ramp day is at least about 0.5 mg.

In certain embodiments, the anti-CD3 antibody or antigen binding fragment thereof is administered on at least one pre-ramp day prior to ramp day one. In certain embodiments, the amount of the anti-CD3 antibody or fragment administered on the at least one pre-ramp day does not exceed 0.3 mg or does not exceed 0.2 mg. In certain embodiments, the amount of anti-CD3 antibody or fragment thereof administered on the at least one pre-ramp day is about 0.1 mg, about 0.2 mg, or about 0.3 mg.

In certain embodiments, an animal administered an anti-CD3 antibody or antigen binding fragment thereof according to a dosing regimen as disclosed herein suffers from an immune-related disease, e.g., a disease selected from the group consisting of: type I diabetes, type II diabetes, psoriasis, rheumatoid arthritis, lupus, inflammatory bowel disease, ulcerative colitis, Crohn's disease, multiple sclerosis, effects of organ transplantation, and graft-versus-host disease (GVHD). In certain embodiments, the animal suffers from diabetes. In certain embodiments, the animal suffers from psoriasis or rheumatoid arthritis. In certain embodiments, the animal is a mammal, e.g. a human.

In certain embodiments, the total amount of antibody or fragment administered is no greater than about 8.6 mg, e.g., no greater than about 6.85 mg or no greater than about 3.1 mg. In certain embodiments, the anti-CD3 antibody or antigen binding fragment thereof is administered intravenously.

In certain embodiments, the anti-CD3 antibody or antigen binding fragment thereof is administered in a single daily dose on at least one day of the dosing regimen, e.g. on each day of the dosing regimen. In certain embodiments, the anti-CD3 antibody or fragment is administered more than once a day on at least one day of the dosing regimen, e.g., on each day of the dosing regimen. In certain embodiments, the interval between administrations is at least one hour. In certain embodiments, the anti-CD3 antibody or fragment is administered over a period of time on at least one day of the dosing regimen, e.g., over a period of at least fifteen minutes.

In certain embodiments, an antigen binding fragment is selected from the group consisting of a Fab fragment, a F(ab′)₂ fragment and a scFv fragment.

In certain embodiments, the anti-CD3 antibody or antigen binding fragment thereof is administered with a pharmaceutically acceptable carrier or diluent. In certain embodiments, the anti-CD3 antibody or fragment is administered in conjunction with another therapeutic agent.

In certain embodiments, the anti-CD3 antibody or antigen binding fragment thereof is chimeric or humanized. In certain embodiments, the antibody is otelixizumab (also referred to herein sometimes as “TRX4”). In certain embodiments, the anti-CD3 antibody or fragment comprises an Fc domain, wherein the Fc domain is aglycosylated. In certain embodiments, the anti-CD3 antibody or fragment comprises an amino acid sequence of SEQ ID NO: 3, an amino acid sequence of SEQ ID NO: 4, or both. In certain embodiments, the anti-CD3 antibody or fragment comprises an alanine at an amino acid position corresponding to amino acid position 299 of SEQ ID NO: 1. In certain embodiments, the antibody is hOKT3, hOKT3γ1(Ala-Ala), HUM291, NI-0401.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a line graph showing the percent of CD4+FoxP3+ T cells compared to baseline in human subjects administered otelixizumab intravenously according to the following 8-day dosing schedule: 0.1 mg on day 1, 0.2 mg on day 2, 0.3 mg on day 3, and 0.75 mg on days 4-8. Each consecutive data point on the line graph corresponds to the consecutive labels on the X axis. Means and standard deviations (SD) are shown. Abbreviations are as follows: Screen=data obtained with samples taken during patient screening, approximately 6-8 weeks prior to treatment. Baseline=data obtained with samples taken immediately prior to the first dose of the dosing regimen. Pre=data obtained with samples taken immediately prior to daily dosing. EOI=end of infusion. The three different lines shown in the graph (CH 2A, CH 2B, and CH2 Lot 2) represent data from studies using the same dosing schedule but different times of infusion and/or different batches of otelixizumab.

FIG. 2 is a line graph showing the percent of CD8+FoxP3+ T cells compared to baseline in human subjects administered otelixizumab intravenously according to the following 8-day dosing schedule: 0.1 mg on day 1, 0.2 mg on day 2, 0.3 mg on day 3, and 0.75 mg on days 4-8. Each consecutive data point on the line graph corresponds to the consecutive labels on the X axis. Means and SD are shown. Abbreviations are as described above for FIG. 1.

FIG. 3 is a line graph showing the percent of CD4+CD25+FoxP3+ T cells compared to baseline in human subjects administered otelixizumab intravenously according to the following 8-day dosing schedule: 0.1 mg on day 1, 0.2 mg on day 2, 0.3 mg on day 3, and 0.75 mg on days 4-8. Means and SD are shown. Each consecutive data point on the line graph corresponds to the consecutive labels on the X axis. Abbreviations are as described above for FIG. 1. The three different lines shown in the graph (CH 2A, CH 2B, and CH2 Lot 2) represent data from studies using the same dosing schedule but different times of infusion and/or different batches of otelixizumab.

FIG. 4 is a line graph showing the amount of cell bound otelixizumab detected by a fluorochrome-conjugated anti-human IgG antibody on CD4+ T cells, expressed in MESF (Molecules of Equivalent Soluble Fluorochrome) units. Subjects in the cohort designated CH2 (indicated by the line with square data points) were administered otelixizumab intravenously according to the following dosing schedule: 0.1 mg on day 1, 0.2 mg on day 2, 0.3 mg on day 3, and 0.75 mg on days 4-8. Subjects in the cohort designated CH3 (indicated by the line with triangle data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Subjects in the cohort designated CH4 (indicated by the line with diamond data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 3.5 mg on day 8 (2×1.75 mg doses). Each consecutive data point on the line graph corresponds to the consecutive labels on the X axis. Means and SD are shown. Abbreviations are as described above for FIG. 1.

FIG. 5 is a line graph showing the number of cell bound otelixizumab molecules on CD4+ T cells of human subjects treated as follows: Subjects in the cohort designated CH2 (indicated by the line with square data points) were administered otelixizumab intravenously according to the following dosing schedule: 0.1 mg on day 1, 0.2 mg on day 2, 0.3 mg on day 3, and 0.75 mg on days 4-8. Subjects in the cohort designated CH3 (indicated by the line with triangle data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Subjects in the cohort designated CH4 (indicated by the line with diamond data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 3.5 mg on day 8 (2×1.75 mg doses). Each consecutive data point on the line graph corresponds to the consecutive labels on the X axis. Means and SD are shown. Abbreviations are as described above for FIG. 1.

FIG. 6 is a line graph showing the percent of CD3/TCR sites detected on CD4+ T cells with a non-competing anti-CD3 antibody. Subjects in the cohort designated CH2 (indicated by the line with square data points) were administered otelixizumab intravenously according to the following dosing schedule: 0.1 mg on day 1, 0.2 mg on day 2, 0.3 mg on day 3, and 0.75 mg on days 4-8. Subjects in the cohort designated CH3 (indicated by the line with triangle data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Subjects in the cohort designated CH4 (indicated by the line with diamond data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 3.5 mg on day 8 (2×1.75 mg doses). Each consecutive data point on the line graph corresponds to the consecutive labels on the X axis. Means and SD are shown. Abbreviations are as described above for FIG. 1.

FIG. 7 is a line graph showing free CD3 sites (i.e., sites recognizable by otelixizumab i.e., sites without otelixizumab bound) on CD4+ T cells as detected with biotinylated otelixizumab and fluoroscein-conjugated streptavidin, expressed in MESF units. Subjects in the cohort designated CH2 (indicated by the line with square data points) were administered otelixizumab intravenously according to the following dosing schedule: 0.1 mg on day 1, 0.2 mg on day 2, 0.3 mg on day 3, and 0.75 mg on days 4-8. Subjects in the cohort designated CH3 (indicated by the line with triangle data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Subjects in the cohort designated CH4 (indicated by the line with diamond data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 3.5 mg on day 8 (2×1.75 mg doses). Each consecutive data point on the line graph corresponds to the consecutive labels on the X axis. Means and SD are shown. Abbreviations are as described above for FIG. 1.

FIG. 8 is a line graph showing absolute counts of CD4+ T cells. Subjects in the cohort designated CH2 (indicated by the line with square data points) were administered otelixizumab intravenously according to the following dosing schedule: 0.1 mg on day 1, 0.2 mg on day 2, 0.3 mg on day 3, and 0.75 mg on days 4-8. Subjects in the cohort designated CH3 (indicated by the line with triangle data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Subjects in the cohort designated CH4 (indicated by the line with diamond data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 3.5 mg on day 8 (2×1.75 mg doses). Each consecutive data point on the line graph corresponds to the consecutive labels on the X axis. Means and SD are shown. Abbreviations are as described above for FIG. 1.

FIG. 9 is a line graph showing absolute counts of CD8+ T cells. Subjects in the cohort designated CH2 (indicated by the line with square data points) were administered otelixizumab intravenously according to the following dosing schedule: 0.1 mg on day 1, 0.2 mg on day 2, 0.3 mg on day 3, and 0.75 mg on days 4-8. Subjects in the cohort designated CH3 (indicated by the line with triangle data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Subjects in the cohort designated CH4 (indicated by the line with diamond data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 3.5 mg on day 8 (2×1.75 mg doses). Each consecutive data point on the line graph corresponds to the consecutive labels on the X axis. Means and SD are shown. Abbreviations are as described above for FIG. 1.

FIG. 10 is a line graph showing the CD3/TCR sites detected on CD4+ T cells with a non-competing anti-CD3 antibody (i.e., an anti-CD3 antibody that does not compete with otelixizumab for binding to CD3). Subjects in the cohort designated CH2 (indicated by the line with square data points) were administered otelixizumab intravenously according to the following dosing schedule: 0.1 mg on day 1, 0.2 mg on day 2, 0.3 mg on day 3, and 0.75 mg on days 4-8. Subjects in the cohort designated CH3 (indicated by the line with triangle data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Subjects in the cohort designated CH4 (indicated by the line with diamond data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 3.5 mg on day 8 (2×1.75 mg doses). Each consecutive data point on the line graph corresponds to the consecutive labels on the X axis. Means and SD are shown. Abbreviations are as described above for FIG. 1.

FIG. 11 is a line graph showing otelixizumab serum concentration. Subjects in the cohort designated CH2 (indicated by the line with square data points) were administered otelixizumab intravenously according to the following dosing schedule: 0.1 mg on day 1, 0.2 mg on day 2, 0.3 mg on day 3, and 0.75 mg on days 4-8. Subjects in the cohort designated CH3 (indicated by the line with triangle data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Subjects in the cohort designated CH4 (indicated by the line with diamond data points) were administered otelixizumab intravenously according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 3.5 mg on day 8 (2×1.75 mg doses). Each consecutive data point on the line graph corresponds to the consecutive labels on the X axis. Abbreviations are as described above for FIG. 1. Means are shown. DD=dose day.

FIG. 12 is a line graph showing the effect of otelixizumab concentration and exposure time on primary MLR responses. PBL (peripheral blood lymphocytes) from normal individuals were separately used as responder cells and combined with stimulator PBL cells from an HLA incompatible normal donor treated with mitomycin C, in the presence of the indicated concentration of otelixizumab (0-1 μg/mL) for the indicated amount of time (2-120 hours). Cells were incubated for 5 days, after which ³H-thymidine was added to each well to measure lymphocyte proliferation. Incorporated ³H was measured by scintillation counting. Results are expressed as the percent of ³H incorporated by the antibody treated samples relative to untreated control wells. Data shown are the cumulative mean values with SD from 6 normal individuals.

FIGS. 13A and 13B are line graphs showing TCR Modulation and Saturation of CD3 Receptors by otelixizumab. PBL from 4 normal individuals were incubated at 37° C. with 0-1 μg/mL of otelixizumab for 2-120 hours in RPMI media with 10% human AB serum. After 5 days, samples were taken and free, unbound CD3 sites (i.e., sites without bound otelixizumab) present on cells were detected with FITC-conjugated otelixizumab (FIG. 13A). In addition, samples were stained with BMA031, an anti-TCR antibody demonstrated not to compete with otelixizumab for binding to CD3 (FIG. 13B). For each staining condition, the mean channel fluorescence (MCF) of the antibody treated cells was compared with the MCF of the control cells to determine the percent of the control level of expression for each reagent. Modulation can be detected as a decrease in TCR expression and a lack of free CD3 sites on cells. Data shown are the cumulative mean values with SD.

FIG. 14 is a line graph showing the effect of otelixizumab concentration on memory MLR responses. PBL (peripheral blood lymphocytes) from normal individuals were separately used as responder cells and cultured with stimulator PBL cells from an HLA incompatible-normal donor treated with mitomycin C for 7 days in the absence of otelixizumab. These cells were then re-stimulated with the original stimulator cells or new (novel) stimulators for 3 days in the presence of otelixizumab. After 3 days of restimulation, ³H-thymidine was added to each well to measure lymphocyte proliferation. Incorporated ³H was measured by scintillation counting. Results are expressed as the percent of ³H incorporated by the otelixizumab treated samples relative to untreated control wells. Data shown are the cumulative mean values with SD from 6 normal individuals.

FIGS. 15A and 15B are line graphs showing modulation of CD3/TCR complex on circulating mouse T-cells during anti-CD3 mAb F(ab′)₂ treatment in Study A (see Example 7). Mean (+/−SD) TCR expression levels are presented as MESF units. (FIG. 15A) BALB/c mice (n=3 per dose regimen) received 50 μg anti-CD3 mAb F(ab′)₂ (or vehicle control) per day for 5 consecutive days. TCR expression was evaluated on circulating CD4+ lymphocytes at 2 hr (post-dose) and 24 hr (pre-dose) after each dose. (FIG. 15B) BALB/c (n=3 per dose regimen) mice received 4 doses of 25, 5, 2, or 1 μg anti-CD3 mAb F(ab′)₂ or vehicle control every 72 hr. TCR expression was evaluated on circulating CD4+ lymphocytes at 2 hr (post-dose) and 72 hr (pre-dose) after each dose. At the pre dose 4 and post dose 4 time-points, differences in TCR expression levels between the 1 and 2 μg dose regimens were significant, p<0.05 and p<0.01 respectively.

FIG. 16 is a bar graph showing lymphocyte counts during the anti-CD3 mAb F(ab′)₂ treatment Study A shown in FIGS. 15A and 15B (Example 7). Complete blood counts were performed 2 hr after the last dose. The lymphocyte count (K/μL) is the mean of 3-5 mice/dose; error bars represent the SD. All dose regimens, with the exception of the 25 μg dose regimen, were significantly different from the vehicle group (*p<0.05). There was no significant difference between the 1 and 2 μg dose regimens.

FIGS. 17A and 17B are bar graphs showing evaluation of lymphocyte populations in peripheral blood of mice treated with CD3 mAb F(ab′)₂ fragments (1, 2, 5, 25, or 50 μg) in Studies B and C (see Example 7). The proportions of CD4+, CD8+, and CD4+FoxP3+ T-cells, measured by flow cytometry, in peripheral blood within 24 hr of the last antibody dose. (FIG. 17A) Mean (±SEM (standard errors of means)) proportions of T-cell subsets in the antibody treatment groups (all dose regimens combined (n=45-51) versus placebo group (n=9). (FIG. 17B) Proportions of T-cell subsets in each group for mice that entered remission vs. mice that remained diabetic (n=2-9 per group).

FIGS. 18A-D show estimation of beta-cell mass of NOD/ShiLtJ mice before and after treatment with CD3 mAb F(ab′)₂ fragments and histologic analyses of pancreata from treated mice that were either in remission or remained diabetic at the end of the study. (FIG. 18A) Comparison of blood glucose measurements prior to initiation of antibody treatment for treated mice that were either in remission (n=47) or remained diabetic (n=32) at study end (mean±SEM). (FIG. 18B) Comparison of serum C-peptide levels before (n=4-5) and 12 weeks after (n=8-9) antibody treatment in treated mice. (FIG. 18C) Representative photograph of peri-insulitis of islet from a mouse treated with 5 μg (4×/72 hr) dose that was in remission at the 12-week study end point. (FIG. 18D) Peri-insulitis scores (PIS) of islets in pancreatic sections from mice in Study B (Example 7) at the 12-week study assessment (diabetic, n=19; remission, n=36).

FIG. 19 is a line graph showing CD3/TCR-complex modulation on circulating T cells during anti-CD3 mAb treatment in a clinical study. Subjects (n=16) were dosed with an 8-day regimen of otelixizumab. TCR expression on circulating CD4+ T-cells was assessed by flow cytometry prior to infusion, at the end of infusion (EOI), and 2 hr after the EOI. Mean (+/−SD) TCR antibody expression levels are presented as MESF units.

FIG. 20 is a line graph showing the number of CD4+CD25+FoxP3+ T cells (Treg cells) during anti-CD3 mAb treatment in a clinical study. Subjects (n=5) were dosed with an 8-day regimen of otelixizumab (TTEDD CH4). Expression of CD4, CD25, and FoxP3 on circulating T-cells was assessed by flow cytometry prior to infusion, at the end of infusion (EOI), and 2 hr after the EOI. Mean (+/−SD). The number of Treg cells are expressed as percent of baseline.

FIG. 21 is a line graph showing the absolute numbers (×10⁹/L) of CD4+CD25+FoxP3+ T cells (Treg cells) during anti-CD3 mAb treatment in a clinical study. Subjects (n=19) were dosed with an 8-day regimen of otelixizumab (TTEDD CH4). Expression of CD4, CD25, and FoxP3 on circulating T-cells was assessed by flow cytometry prior to infusion, at the end of infusion (EOI), and 2 hr after the EOI. Mean (+/−SD).

FIG. 22 is a line graph showing the level of cell-bound otelixizumab on CD4+ T cells expressed as standard MESF units in age groups 17 and younger (square symbol) and 18 and older (triangle symbol). The line with the diamond symbol indicates the average of the two age groups. Subjects (n=13) were dosed with a 5-day regimen of otelixizumab (TTEDD CH5).

FIG. 23 is a line graph showing CD3/TCR-complex modulation on circulating T cells during anti-CD3 mAb treatment in a clinical study. Subjects (n=13) were dosed with a 5-day regimen of otelixizumab (TTEDD CH5). TCR expression on circulating CD4+ T-cells was assessed by flow cytometry prior to infusion, at the end of infusion (EOI), and 2 hr after the EOI. Mean (+/−SD) TCR antibody expression levels are presented as percent of baseline in age groups 17 and younger (square symbol) and 18 and older (triangle symbol). The line with the diamond symbol indicates the average of the two age groups.

FIG. 24 is a line graph showing the level of free CD3 sites on CD4+ T cells detected by biotinylated otelixizumab and expressed as standard MESF units in age groups 17 and younger (square symbol) and 18 and older (triangle symbol). The line with the diamond symbol indicates the average of the two age groups. Subjects (n=13) were dosed with a 5-day regimen of otelixizumab (TTEDD CH5).

FIG. 25 is a line graph showing the absolute numbers (×10⁹/L) of CD4+ T cells during anti-CD3 mAb treatment in a clinical study in age groups 17 and younger (square symbol) and 18 and older (triangle symbol). The line with the diamond symbol indicates the average of the two age groups. Subjects (n=13) were dosed with a 5-day regimen of otelixizumab (TTEDD CH5). Expression of CD4 on circulating T-cells was assessed by flow cytometry prior to infusion, at the end of infusion (EOI), and 2 hr after the EOI. Mean (+/−SD).

FIG. 26 is a line graph showing the absolute numbers (×10⁹/L) of CD8+ T cells during anti-CD3 mAb treatment in a clinical study in age groups 17 and younger (square symbol) and 18 and older (triangle symbol). The line with the diamond symbol indicates the average of the two age groups. Subjects (n=13) were dosed with a 5-day regimen of otelixizumab (TTEDD CH5). Expression of CD8 on circulating T-cells was assessed by flow cytometry prior to infusion, at the end of infusion (EOI), and 2 hr after the EOI. Mean (+/−SD).

FIG. 27 is a line graph showing the serum concentration (μg/ml) of otelixizumab during anti-CD3 mAb treatment in a clinical study in age groups 17 or 18 and younger (diamond symbol, “Adolescence Avg”) (n=8) and 17 or 18 and older (square symbol, “Adult Avg”) (n=10). The line with no symbol indicates the limit of quantitation (“LOQ”). Subjects were dosed with a 5-day regimen of otelixizumab (TTEDD CH5). Serum concentration was assessed by ELISA prior to infusion, at the end of infusion (EOI), and 2 hr after the EOI. Mean (+/−SD).

FIG. 28 is a line graph showing the Cₘᵢₙ and Cₘₐₓ for each daily dose of otelixizumab in Cohort C(RT-C).

FIG. 29 is a line graph showing the Cₘᵢₙ and Cₘₐₓ for each daily dose of otelixizumab in TTEDD CH1.

FIG. 30 is a line graph showing the Cₘᵢₙ and Cₘₐₓ for each daily dose of otelixizumab in TTEDD CH2.

FIG. 31 is a line graph showing the Cₘᵢₙ and Cₘₐₓ for each daily dose of otelixizumab in TTEDD CH3.

FIG. 32 is a line graph showing the Cₘᵢₙ and Cₘₐₓ for each daily dose of otelixizumab in TTEDD CH4.

FIG. 33 is a line graph showing the Cₘᵢₙ and Cₘₐₓ for each daily dose of otelixizumab in TTEDD CH5.

FIG. 34 is a line graph showing the Cₘᵢₙ and Cₘₐₓ for each daily dose of otelixizumab in BDR Group B. The otelixizumab half-life equals 1.52 day and volume of distribution is 7.56 L. The maximal and minimal concentrations for a typical subject were calculated using eq. (6) (Example 8). The dosing scheme was 24, 8.0, 8.0, 8.0, 8.0, and 8.0 (mg).

FIG. 35 is a line graph showing the Cₘᵢₙ and Cₘₐₓ for each daily dose of otelixizumab in BDR Group B. The otelixizumab half-life equals 1.52 day and volume of distribution 7.56 L. The maximal and minimal concentrations for a typical subject were calculated using eq. (6) (Example 8). The dosing scheme was 8.0, 8.0, 8.0, 8.0, 8.0, and 8.0 (mg).

FIG. 36 is a line graph showing the maximum and minimum levels of free receptors (FR) and drug-bound receptors (DR) on CD4+ and CD8+ T cells for each daily dose of otelixizumab in Cohort C.

FIG. 37 is a line graph showing the maximum and minimum levels of free receptors (FR) and drug-bound receptors (DR) on CD4+ and CD8+ T cells for each daily dose of otelixizumab in TTEDD CH 1.

FIG. 38 is a line graph showing the maximum and minimum levels of free receptors (FR) and drug-bound receptors (DR) on CD4+ and CD8+ T cells for each daily dose of otelixizumab in TTEDD CH2.

FIG. 39 is a line graph showing the maximum and minimum levels of free receptors (FR) and drug-bound receptors (DR) on CD4+ and CD8+ T cells for each daily dose of otelixizumab in TTEDD CH3.

FIG. 40 is a line graph showing the maximum and minimum levels of free receptors (FR) and drug-bound receptors (DR) on CD4+ and CD8+ T cells for each daily dose of otelixizumab in TTEDD CH4.

FIG. 41 is a line graph showing the maximum and minimum levels of free receptors (FR) and drug-bound receptors (DR) on CD4+ and CD8+ T cells for each daily dose of otelixizumab in TTEDD CH5.

FIG. 42 is a line graph showing the maximum and minimum levels of free receptors (FR) and drug-bound receptors (DR) on CD4+ and CD8+ T cells for each daily dose of otelixizumab in BDR, Group A.

FIG. 43 is a line graph showing the maximum and minimum levels of free receptors (FR) and drug-bound receptors (DR) on CD4+ and CD8+ T cells for each daily dose of otelixizumab in BDR, Group B.

FIG. 44 is a line graph showing the level of free receptors on CD4+ and CD8+ T cells and indicating the levels of 10%, 20%, 30%, and 40% of baseline values after various daily doses of otelixizumab in Cohort C.

FIG. 45 is a line graph showing the level of free receptors on CD4+ and CD8+ T cells and indicating the levels of 10%, 20%, 30%, and 40% of baseline values after various daily doses of otelixizumab in TTEDD CH1.

FIG. 46 is a line graph showing the level of free receptors on CD4+ and CD8+ T cells and indicating the levels of 10%, 20%, 30%, and 40% of baseline values after various daily doses of otelixizumab in TTEDD CH2.

FIG. 47 is a line graph showing the level of free receptors on CD4+ and CD8+ T cells and indicating the levels of 10%, 20%, 30%, and 40% of baseline values after various daily doses of otelixizumab in TTEDD CH3.

FIG. 48 is a line graph showing the level of free receptors on CD4+ and CD8+ T cells and indicating the levels of 10%, 20%, 30%, and 40% of baseline values after various daily doses of otelixizumab in TTEDD CH4.

FIG. 49 is a line graph showing the level of free receptors on CD4+ and CD8+ T cells and indicating the levels of 10%, 20%, 30%, and 40% of baseline values after various daily doses of otelixizumab in TTEDD CH5.

FIG. 50 is a line graph showing the level of free receptors on CD4+ and CD8+ T cells and indicating the levels of 10%, 20%, 30%, and 40% of baseline values after various daily doses of otelixizumab in Study II, Cohort 3 (1 dose of 4 mg).

FIG. 51 is a line graph showing the level of free receptors on CD4+ and CD8+ T cells and indicating the levels of 10%, 20%, 30%, and 40% of baseline values after various daily doses of otelixizumab in BDR, Group B.

FIG. 52 is a line graph showing the time in days for which CD4+ and CD8+ T cells had surface levels of free receptors (FR) of 10% to 40% and 20% to 30% of baseline levels after various daily doses of otelixizumab in Cohort C.

FIG. 53 is a line graph showing the time in days for which CD4+ and CD8+ T cells had surface levels of free receptors (FR) of 10% to 40% and 20% to 30% of baseline levels after various daily doses of otelixizumab in TTEDD CH1.

FIG. 54 is a line graph showing the time in days for which CD4+ and CD8+ T cells had surface levels of free receptors (FR) of 10% to 40% and 20% to 30% of baseline levels after various daily doses of otelixizumab in TTEDD CH2.

FIG. 55 is a line graph showing the time in days for which CD4+ and CD8+ T cells had surface levels of free receptors (FR) of 10% to 40% and 20% to 30% of baseline levels after various daily doses of otelixizumab in TTEDD CH3.

FIG. 56 is a line graph showing the time in days for which CD4+ and CD8+ T cells had surface levels of free receptors (FR) of 10% to 40% and 20% to 30% of baseline levels after various daily doses of otelixizumab in TTEDD CH4.

FIG. 57 is a line graph showing the time in days for which CD4+ and CD8+ T cells had surface levels of free receptors (FR) of 10% to 40% and 20% to 30% of baseline levels after various daily doses of otelixizumab in TTEDD CH5.

FIG. 58 is a line graph showing the time in days for which CD4+ and CD8+ T cells had surface levels of free receptors (FR) of 10% to 40% and 20% to 30% of baseline levels after various daily doses of otelixizumab in Study II, Cohort 3 (1 dose of 4 mg).

FIG. 59 is a line graph showing the time in days for which CD4+ and CD8+ T cells had surface levels of free receptors (FR) of 10% to 40% and 20% to 30% of baseline levels after various daily doses of otelixizumab in BDR, Group B.

### DESCRIPTION OF CERTAIN EMBODIMENTS

Provided herein are methods of administering anti-CD3 antibodies or antigen binding fragments thereof to an animal. Methods disclosed herein permit administration of higher cumulative doses of the anti-CD3 antibody or fragment with decreased pro-inflammatory cytokine release and immunogenicity, and with minimal to no perturbation of Epstein Barr Virus immunity. In certain embodiments, methods disclosed herein facilitate higher individual doses later in a dosing regimen than would be possible with traditional dosing regimens.

### DEFINITIONS

“Antibody” as the term is used herein refers to a protein that generally comprises heavy chain polypeptides and light chain polypeptides. IgG, IgD, and IgE antibodies comprise two heavy chain polypeptides and two light chain polypeptides. IgA antibodies comprise two or four of each chain and IgM generally comprises 10 of each chain. Single domain antibodies having one heavy chain and one light chain and heavy chain antibodies devoid of light chains are also contemplated. A given antibody comprises one of five types of heavy chains, called alpha, delta, epsilon, gamma and mu, the categorization of which is based on the amino acid sequence of the heavy chain constant region. These different types of heavy chains give rise to five classes of antibodies, IgA (including IgA1 and IgA2), IgD, IgE, IgG (IgG1, IgG2, IgG3 and IgG4) and IgM, respectively. A given antibody also comprises one of two types of light chains, called kappa or lambda, the categorization of which is based on the amino acid sequence of the light chain constant domains.

“Antigen binding fragment”, “antigen binding antibody fragment”, and “fragment” as the terms are used herein refer to an antigen binding molecule that is not an antibody as defined above, but that has at least one antigen binding site of an antibody. Thus an antigen binding fragment or antigen binding antibody fragment of an anti-CD3 antibody is a fragment of an antibody that binds to CD3, and also can be referred to herein as a “CD3-binding fragment.” Antigen binding fragments often comprise a cleaved portion of a whole antibody, although the term is not limited to such cleaved fragments. Antigen binding fragments can include, for example, Fab fragments, F(ab′)₂ fragments, scFv (single chain Fv) fragments, diabodies, linear antibodies, multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and V_{HH} containing antibodies.

“Humanized antibody” as the term is used herein refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those of ordinary skill in the art will be aware of humanized antibodies, and will also be aware of suitable techniques for their generation.

“Chimeric antibody” as the term is used herein refers to an antibody that has been engineered to comprise a human constant region. Chimeric antibodies are typically less immunogenic to humans, relative to non-chimeric antibodies, and thus offer therapeutic benefits in certain situations. Those of ordinary skill in the art will be aware of chimeric antibodies, and will also be aware of suitable techniques for their generation.

“Dosing regimen,” “regimen” and “antibody dosing regimen,” as the terms are used herein, refer to the total course of treatment administered to an animal, e.g., treatment with an anti-CD3 antibody or antigen binding fragment thereof. In some embodiments, the total amount of the anti-CD3 antibody or fragment administered to the patient does not exceed 300 μg/kg when administered intravenously, and when administered other than intravenously, the total amount administered does not exceed the bioequivalent of intravenous administration of 300 μg/kg.

A dosing regimen may include a given number of days of treatment. For example, an anti-CD3 dosing regimen may include administering an anti-CD3 antibody to an animal for a minimum number of days, a maximum number of days, or a specific number of days. As non-limiting examples, an anti-CD3 antibody may be administered to an animal over a regimen of five days, eight days, or any number of days in between or beyond. An anti-CD3 dosing regimen may be as short as one day, although as will be apparent from the remainder of the present specification, multiple day dosing regimens permit administration of higher amounts of antibody on later days while significantly reducing cytokine release syndrome and other negative effects. Regimens are generally 21 days or less (e.g., 18 days or less, 14 days or less, 12 days or less, 10 days or less, 8 days or less, 5 days or less, 3 days or less, 2 days or less, or 1 day) in length. Regimens can be separated by relatively short periods of time (e.g., 5 days, 10 days, 15 days, 20 days, 25 days, 30 days, 1.5 months, 2 months, 3 months, or 4 months) or longer periods of time (e.g., 6 months, 9 months, 12 months, 18 months. 2 years, 3 years, 4 years, 5 years, 10 years, 15 years, or 20 years). Additionally and/or alternatively, a regimen may include a given amount of therapeutic agent administered per day. For example, an anti-CD3 antibody or fragment may be administered to an animal in a minimum amount on one or more days of the regimen, in a maximum amount on one or more days of the regimen, or in a specific amount on one or more days of the regimen.

As used herein, the term “therapy window” refers to the time period starting on the first day of a dosing regimen and extending past the last day of the dosing regimen to the first time at which no anti-CD3 antibody or antigen binding fragment thereof is detectable (using a standard ELISA assay) in the peripheral blood plasma of the human undergoing the relevant dosing regimen.

As used herein, the term “continuous” in the context of the time in which the mean level of free CD3/TCR complexes on appropriate T cells is within a specific range of levels, means that the time the mean level is in that specific range is not interrupted by any time in which that mean level is not within that specific range of levels.

As used herein, the term “not continuous” in the context of the time in which the mean level of free CD3/TCR complexes on appropriate T cells is within a specific range of levels, means that the time the mean level is in that specific range is interrupted by some amount of time (e.g., 15 minutes, 20 minutes, 30 minutes, 45 minutes, 1 hour, 2 hours, 3 hours, 4, hours, 5 hours, 6 hours, 8 hours, 10 hours, 12 hours, 14 hours, 16 hours 18 hours, 20 hours, 24 hours 28 hours, 32 hours, 36 hours, 40 hours, 44 hours, 48 hours, 60 hours, 72 hours, 84 hours, 90 hours, or any range of time of having upper and lower limits of any of above the specifically stated times), in which that mean level is not within that specific range of levels.

### Fc Receptors

In certain embodiments, the anti-CD3 antibodies and antigen binding fragments thereof do not bind or have reduced binding to at least one class of Fc (gamma) receptor. The Fc receptors are a family of cell-surface molecules that bind the Fc portion of immunoglobulins. Each member of the family recognizes immunoglobulin of one isotype or a few closely related isotypes through a recognition domain on the alpha chain of the Fc receptor. Fc receptors are themselves members of the immunoglobulin superfamily. Different accessory cells bear Fc receptors for antibodies of different isotypes, and the isotype of the antibody thus determines which accessory cell will be engaged in a given response. There are at least four types of Fc receptors, including those belonging to the gamma (e.g., Fc (gamma) RI), epsilon (e.g., Fc (epsilon) RIa) and alpha (e.g., Fc (alpha) RI) groups, as well as the neonatal FcR (FcRn). FcRn transports IgG molecules across the placenta in humans and also across the gut in rats and mice. FcRn is also involved in the homeostasis of IgG in humans. Fc (epsilon) RI binds IgE with high affinity, Fc (alpha) RI binds IgA, and Fc (gamma) receptors bind IgG. The Fc (gamma) receptor group is further divided into classes, which include at least Fc (gamma) RI, Fc (gamma) RII-A, Fc (gamma) RII-C, Fc (gamma) RII-B2, Fc (gamma) RII-B1, Fc (gamma) RIIIA, Fc (gamma) RIIIB, and Fc (gamma) RIV. These classes of Fc (gamma) receptors can vary in the types of cells on which they are expressed, the effects of their ligation (e.g., inhibitory or activating), and their affinity for the Fc of different antibody isotypes. For example, the affinity of Fc (gamma) RI for IgG1 is about 10⁸ M⁻¹; the affinities of Fc (gamma) RII-A, RII-B2 and RII-B1 for IgG1 are each about 2×10⁶ M⁻¹; and the affinity of Fc (gamma) RIII is about 5×10⁵ M⁻¹. A detailed description of the Fc receptors is provided in Janeway, C. A. et al. Immunobiology; The Immune System in Health and Disease; (2001) 5^{th} edition; Garland Publishing, New York, N.Y.; see, e.g., pages 362-363 and 370-377; and a detailed description of Fc (gamma) receptors is provided in Nimmerjahn and Ravetch; “Fcgamma receptors as regulator of immune responses”; Nat Rev Immunol. 2008 January; 8(1):34-47, the disclosures of which are incorporated herein by reference in their entirety.

### Exemplary Dosing Regimens

Provided herein are methods of administering anti-CD3 antibodies or antigen binding fragments thereof to an animal. In certain embodiments, the anti-CD3 antibody or fragment to be administered does not bind or has reduced binding to at least one class of Fc (gamma) receptor. For example, an anti-CD3 antibody or fragment may have reduced binding to at least one class of Fc (gamma) receptor as compared to the OKT3 antibody. As another example, an anti-CD3 antibody or fragment may have reduced binding to at least one class of Fc (gamma) receptor as compared to the huOKT3-gamma-1 and/or huOKT3-gamma-1(A³¹⁸) antibodies as described in Xu et al., Cellular Immunology, 200, 16-26 (2000), incorporated herein by reference in its entirety. As another example, an anti-CD3 antibody or fragment may have reduced binding to at least one class of Fc (gamma) receptor as compared to the IgG1 immunoglobulin produced by the ARH-77 cell line deposited under ATCC catalog number CRL-1621.

Methods disclosed herein, including but not limited to methods disclosed in this section, permit administration of higher cumulative doses of the anti-CD3 antibody or antigen binding fragment thereof with decreased pro-inflammatory cytokine release and immunogenicity, and with minimal to no perturbation of Epstein Barr Virus immunity. In certain embodiments, methods disclosed herein, including but not limited to methods disclosed in this section, facilitate higher individual doses later in a dosing regimen than would be possible with traditional dosing regimens.

In certain embodiments, the anti-CD3 antibody or antigen binding fragment thereof may be administered over a dosing regimen of one day, two days, three days, four days, five days, six days, seven days, eight days, nine days, ten days, eleven days, twelve days, thirteen days, fourteen days, or more. In certain embodiments, the anti-CD3 antibody or fragment is administered over a dosing regimen of five days. In certain embodiments, the anti-CD3 antibody or fragment is administered over a dosing regimen of eight days. In certain embodiments, the anti-CD3 antibody or fragment is administered as a continuous infusion (e.g., by a microinfusion pump or slow-release patch) rather than a fixed dose. Limiting the number of days of a dosing regimen can confer practical benefits on a patient being treated. For example, limiting a dosing regimen to five days may minimize the inconvenience to a patient when that patient needs to travel to a hospital or clinic to receive anti-CD3 antibody or fragment treatment. Limiting the number of days in a dosing regimen can also increase patient safety since fewer hospital visits will result in fewer medical recordkeeping requirements, and thus fewer chances of making recording or filing mistakes. Limiting the number of days in a given dosing regimen can also decrease the costs associated with treatment, since the treatment provider will need to spend less total time with the patient.

In certain embodiments, an anti-CD3 antibody or antigen binding fragment thereof is administered on consecutive days during a given dosing regimen. In certain embodiments, the anti-CD3 antibody or fragment is not administered on consecutive days of a dosing regimen. For example, a given dosing regimen may include one or more days in which the anti-CD3 antibody or fragment is not administered. In certain embodiments, a dosing regimen comprises one, two, three, four, five, six, seven or more days in which the anti-CD3 antibody or fragment is not administered. In certain embodiments, the anti-CD3 antibody or fragment is administered every other day of a dosing regimen. In certain embodiments, the anti-CD3 antibody or fragment is administered every third day, or every fourth day.

In certain embodiments, an anti-CD3 antibody or antigen binding fragment thereof is administered in a low dose on at least one day of a dosing regimen. In certain embodiments, the anti-CD3 antibody or fragment is administered in a low dose during the early portion of a dosing regimen, e.g., on the first one, two and/or three days of the regimen. As will be appreciated by those of ordinary skill in the art upon reading the present specification, administering the anti-CD3 antibody or fragment in a low dose during the early portion of a dosing regimen facilitates the administration of higher individual doses later in a dosing regimen than would be possible with traditional dosing regimens. In certain embodiments, the anti-CD3 antibody or fragment is administered in an amount that does not exceed about 0.5 mg per day during the early portion of a dosing regimen. For example, the anti-CD3 antibody or fragment may be administered in an amount that does not exceed about 0.5 mg per day on the first one, two and/or three days of the regimen. In certain embodiments, the amount of the anti-CD3 antibody or fragment administered on the first two days of the dosing regimen does not exceed about 0.5 mg per day. In certain embodiments, the amount of the anti-CD3 antibody or fragment administered on the first day of the dosing regimen does not exceed about 0.5 mg. In certain embodiments, the anti-CD3 antibody or fragment is administered in an amount that does not exceed about 0.45 mg per day, about 0.4 mg per day, about 0.35 mg per day, about 0.3 mg per day, about 0.25 mg per day, about 0.2 mg per day, about 0.15 mg per day, about 0.1 mg per day, about 0.09 mg per day, about 0.08 mg per day, about 0.07 mg per day, about 0.06 mg per day, about 0.05 mg per day, about 0.04 mg per day, about 0.03 mg per day, about 0.02 mg per day, about 0.01 mg per day, or less during the early portion of a dosing regimen, e.g. on the first one, two and/or three days of the regimen.

In certain embodiments, the amount of the anti-CD3 antibody or antigen binding fragment thereof administered on each of days one and two of a given dosing regimen does not exceed about 0.3 mg per day. In certain embodiments, the amount of the anti-CD3 antibody or fragment administered on each of days one and two of a given dosing regimen does not exceed about 0.2 mg per day. In certain embodiments, the amount of the anti-CD3 antibody or fragment administered on day one of a given dosing regimen is about 0.1 mg. In certain embodiments, the amount of anti-CD3 antibody or fragment administered on day two of a given dosing regimen is about 0.2 mg. In certain embodiments, the amount of the anti-CD3 antibody or fragment administered on day two of a given dosing regimen is about 0.3 mg.

In certain embodiments, the amount of anti-CD3 antibody or antigen binding fragment thereof administered increases between days two and five of a given dosing regimen. In certain embodiments, the amount of increase between days two and five is more than about 0.3 mg. For example, the amount of the anti-CD3 antibody or fragment administered may increase more than about 0.3 mg, more than about 0.35 mg, more than about 0.4 mg, more than about 0.45 mg, more than about 0.5 mg, more than about 0.55 mg, more than about 0.6 mg, more than about 0.65 mg, more than about 0.7 mg, more than about 0.75 mg, more than about 0.8 mg, more than about 0.85 mg, more than about 0.9 mg, more than about 0.95 mg, more than about 1.0 mg, more than about 1.1 mg, more than about 1.2 mg, more than about 1.3 mg, more than about 1.4 mg, more than about 1.5 mg, more than about 1.6 mg, more than about 1.7 mg, more than about 1.8 mg, more than about 1.9 mg, more than about 2 mg, more than about 2.5 mg, more than about 3 mg, more than about 3.5 mg, more than about 4 mg, more than about 4.5 mg, more than about 5 mg, or more.

In certain embodiments, the amount of anti-CD3 antibody or antigen binding fragment thereof administered increases on each day between days two and five of a given dosing regimen such that the total increase between days two and five is more than about 0.3 mg. In certain embodiments, the amount of the anti-CD3 antibody or fragment administered between days two and five of a given dosing regimen increases by more than about 0.3 mg, but the amount of the anti-CD3 antibody or fragment administered does not increase on each day. For example, the amount of the anti-CD3 antibody or fragment administered may remain constant or even decrease between, e.g., days two and three, days three and four, or days four and five, but the total amount nevertheless increases by more than about 0.3 mg between days two and five.

In certain embodiments, the amount of anti-CD3 antibody or antigen binding fragment thereof administered on day three of a given dosing regimen is less than about 0.5 mg greater than the amount of the anti-CD3 antibody or fragment administered on day two of the dosing regimen. For example, the amount of the anti-CD3 antibody or fragment administered on day three of the dosing regimen may be less than about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on day two. In certain embodiments, the amount of the anti-CD3 antibody or fragment administered on day three of the dosing regimen is about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on day two. In certain embodiments, the amount of the anti-CD3 antibody or fragment administered on day three of the dosing regimen is about equal to the amount administered on day two. In certain embodiments, the amount of the anti-CD3 antibody or fragment administered on day three of the dosing regimen is less than the amount administered on day two. For example, the amount of the anti-CD3 antibody or fragment administered on day three of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on day two. In certain embodiments, the amount of the anti-CD3 antibody or fragment administered on day three of the dosing regimen is more than about 0.5 mg less than the amount administered on day two.

In certain embodiments, the amount of anti-CD3 antibody or antigen binding fragment thereof administered on day four of a given dosing regimen is less than about 0.55 mg greater than the amount of the anti-CD3 antibody or fragment administered on day three of the dosing regimen. For example, the amount of the anti-CD3 antibody or fragment administered on day four of the dosing regimen may be less than about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on day three. In certain embodiments, the amount of the anti-CD3 antibody or fragment administered on day four of the dosing regimen is about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on day three. In certain embodiments, the amount of the anti-CD3 antibody or fragment administered on day four of the dosing regimen is about equal to the amount administered on day three. For example, the amount of the anti-CD3 antibody or fragment administered on day four of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on day three. In certain embodiments, the amount of the anti-CD3 antibody or fragment administered on day four of the dosing regimen is more than about 0.5 mg less than the amount administered on day three.

In certain embodiments, the amount of anti-CD3 antibody or antigen binding fragment thereof administered on day five of a given dosing regimen is less than about 0.6 mg greater than the amount of anti-CD3 antibody or fragment administered on day four of the dosing regimen. For example, the amount of the anti-CD3 antibody or fragment administered on day five of the dosing regimen may be less than about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on day four. In certain embodiments, the amount of the anti-CD3 antibody or fragment administered on day five of the dosing regimen is about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on day four. In certain embodiments, the amount of the anti-CD3 antibody or fragment administered on day five of the dosing regimen is about equal to the amount administered on day four. In certain embodiments, the amount of the anti-CD3 antibody or fragment administered on day five of the dosing regimen is less than the amount administered on day four. For example, the amount of the anti-CD3 antibody or fragment administered on day five of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on day four. In certain embodiments, the amount of the anti-CD3 antibody or fragment administered on day five of the dosing regimen is more than about 0.5 mg less than the amount administered on day four.

In certain embodiments, the amount of anti-CD3 antibody or antigen binding fragment thereof administered on day five of a given dosing regimen is at least about 0.5 mg. For example, the amount of the anti-CD3 antibody or fragment administered on day five of a given dosing regimen can be at least about 0.5 mg, at least about 0.55 mg, at least about 0.6 mg, at least about 0.65 mg, at least about 0.7 mg, at least about 0.75 mg, at least about 0.8 mg, at least about 0.85 mg, at least about 0.9 mg, at least about 0.95 mg, at least about 1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2 mg, at least about 2.5 mg, at least about 3 mg, at least about 3.5 mg, at least about 4 mg, at least about 4.5 mg, at least about 5 mg, or higher. In certain embodiments, the amount of the anti-CD3 antibody or fragment administered on day five of a given dosing regimen is about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.2 mg, about 1.3 mg, about 1.4 mg, about 1.5 mg, about 1.6 mg, about 1.7 mg, about 1.8 mg, about 1.9 mg, about 2 mg, about 2.5 mg, about 3 mg, about 3.5 mg, about 4 mg, about 4.5 mg, about 5 mg, or higher.

In certain embodiments, an anti-CD3 antibody or antigen binding fragment thereof is administered according to the following dosing regimen: about 0.1 mg on day one, about 0.2 mg on day two, about 0.3 mg on day three, and about 0.5 mg on each of days four through eight. In certain embodiments, the anti-CD3 antibody or fragment is administered according to the following dosing regimen: about 0.1 mg on day one, about 0.2 mg on day two, about 0.3 mg on day three, and about 0.75 mg on each of days four through eight. In certain embodiments, the anti-CD3 antibody or fragment is administered according to the following dosing regimen: about 0.1 mg on day one, about 0.2 mg on day two, about 0.3 mg on day three, about 0.75 mg day four, about 1.0 mg on day five, about 1.25 mg on day six, about 1.5 mg on day seven, and about 1.75 mg on day eight. In certain embodiments, the anti-CD3 antibody or fragment is administered according to the following dosing regimen: about 0.1 mg on day one, about 0.2 mg on day two, about 0.3 mg on day three, about 0.75 mg day four, about 1.0 mg on day five, about 1.25 mg on day six, about 1.5 mg on day seven, and about 3.75 mg on day eight. In certain embodiments, the anti-CD3 antibody or fragment is administered according to the following dosing regimen: about 0.1 mg on day one, about 0.3 mg on day two, about 0.5 mg on day three, about 0.9 mg on day four, and about 1.3 mg on day five. In certain embodiments, the anti-CD3 antibody or fragment is administered according to the following dosing regimen: about 0.2 mg on day one, about 0.4 mg on day two, about 0.6 mg on day three, about 0.8 mg on day four, and about 1.1 mg on day five. In certain embodiments, the anti-CD3 antibody or fragment is administered according to the following dosing regimen: about 0.2 mg on day one, about 0.4 mg on day two, about 0.8 mg on day three, about 1.4 mg on day four, and about 1.6 mg on day five. In certain embodiments, the anti-CD3 antibody or fragment is administered according to the following dosing regimen: about 0.1 mg on day one, about 0.3 mg on day two, about 0.6 mg on day three, about 1.2 mg on day four, and about 2.2 mg on day five.

In certain embodiments, the anti-CD3 antibody or antigen binding antibody fragment is administered in multiple doses on one or more days of any of the above-described dosing regimens. For example, the anti-CD3 antibody or fragment may be administered in two doses on day eight of a given dosing regimen to achieve a total daily dose of 3.75 mg or more.

In certain embodiments, the total amount of the anti-CD3 antibody or antigen-binding fragment thereof administered to the patient does not exceed 300 μg/kg when administered intravenously, and when administered other than intravenously, the total amount administered does not exceed the bioequivalent of intravenous administration of 300 μg/kg.

In certain embodiments, the total amount of the anti-CD3 antibody or antigen binding fragment thereof administered over the course of a dosing regimen is no greater than about 21 mg. For example, the total amount of the anti-CD3 antibody or fragment administered to a patient over the course of a dosing regimen may no greater than about 21 mg, about 20 mg, about 19 mg, about 18 mg, about 17 mg, about 16 mg, about 15 mg, about 14 mg, about 13 mg, about 12 mg, about 11.5 mg, about 11 mg, about 10.5 mg, about 10 mg, about 9.5 mg, about 9 mg, about 8.5 mg, about 8 mg, about 7.5 mg, about 7 mg, about 6.5 mg, about 6 mg, about 5.5 mg, about 5 mg, about 4.5 mg, about 4 mg, about 3.9 mg, about 3.8 mg, about 3.7 mg, about 3.6 mg, about 3.5 mg, about 3.4 mg, about 3.3 mg, about 3.2 mg, about 3.1 mg, about 3 mg, about 2.9 mg, about 2.8 mg, about 2.7 mg, about 2.6 mg, about 2.5 mg, about 2.4 mg, about 2.3 mg, about 2.2 mg, about 2.1 mg, about 2 mg, about 1.9 mg, about 1.8 mg, about 1.7 mg, about 1.6 mg, about 1.5 mg, 1.4 mg, 1.3 mg, 1.2 mg, 1.1 mg, 1 mg, or less. In certain embodiments, the total amount of the anti-CD3 antibody or fragment administered over the course of a dosing regimen is no greater than about 8.6 mg. In certain embodiments, the total amount of the anti-CD3 antibody or fragment administered over the course of a dosing regimen is no greater than about 6.85 mg. In certain embodiments, the total amount of the anti-CD3 antibody or fragment administered over the course of a dosing regimen is no greater than about 3.1 mg.

Any method of administration may be used to administer anti-CD3 antibodies or antigen binding fragments thereof to a patient. In certain embodiments, the anti-CD3 antibody or fragment is administered to a patient intravenously. In certain embodiments, the anti-CD3 antibody or fragment is administered to a patient by a route other than an intravenous route. For example, the anti-CD3antibody or fragment may be administered to a patient orally, rectally, intramuscularly, intravenously, intranasally, subcutaneously, intraocularly, transdermally, by direct injection into an affected organ or tissue site, or inhaled. In certain embodiments, the anti-CD3 antibody or fragment is administered as a continuous infusion (e.g., by a microinfusion pump or slow-release patch) rather than a fixed dose. In some embodiments, the patient self-administers the antibody or fragment. Those of ordinary skill in the art will be aware of suitable routes of administration and will be able to adapt such routes of administration to any of the dosing regimens disclosed herein.

In certain embodiments, an anti-CD3 antibody or antigen binding fragment thereof is administered in a single daily dose on at least one day of a dosing regimen. In certain embodiments, the anti-CD3 antibody or fragment is administered in a single daily dose on each day of a dosing regimen. A single daily dose of the anti-CD3 antibody or fragment may be administered over a relatively short period of time, e.g., within a period of less than about fifteen minutes. Such embodiments minimize the hospital time and inconvenience to a patient. Alternatively, a single daily dose may be administered to a patient over a longer period of time, e.g., over a period of greater than fifteen minutes. For example, a single daily dose may be administered to a patient over a period of fifteen minutes, thirty minutes, forty-five minutes, one hour, two hours, three hours, four hours, five hours, six hours, seven hours, eight hours, nine hours, ten hours, eleven hours, twelve hours, or more. Such embodiments are useful when, for example, the patient experiences adverse side effects from administering the anti-CD3 antibody or fragment over a relatively short period of time. Administration of the anti-CD3 antibody or fragment to a patient over a period of time may be accomplished in any of a variety of ways such as, without limitation, intravenous administration.

In certain embodiments, a patient may receive more than one course of treatment with the same or different regimen of dosing with the anti-CD3 antibody or antigen binding fragment.

In certain embodiments, an anti-CD3 antibody or antigen binding fragment thereof is administered more than once a day on at least one day of a dosing regimen. In certain embodiments, the anti-CD3 antibody or fragment is administered more than once a day on each day of a dosing regimen. For example, the anti-CD3antibody or fragment can be administered twice, three times or four times on at least one day, or each day, of a dosing regimen. In such embodiments, there will typically be an interval between daily doses. For example, the interval between daily doses can be 1 hour, 2 hours, three hours, four hours, five hours, six hours, seven hours, eight hours, nine hours, ten hours, eleven hours, twelve hour or more. Such embodiments are useful when, for example, the patient experiences adverse side effects from administration of the anti-CD3 antibody or fragment in a single daily dose.

In certain embodiments, methods disclosed herein can be used to treat any of a variety of disease conditions (e.g., immune-related diseases such as any of those described below) in humans. Methods disclosed in the present specification may also be used in the treatment of any of a variety of disease conditions (e.g., immune-related diseases) in non-human animals. Accordingly, doses and methods of administration may be selected in accordance with known principles of veterinary pharmacology and medicine. Guidance may be found, for example, in Adams, R. (ed.), Veterinary Pharmacology and Therapeutics, 8.sup.th edition, Iowa State University Press; ISBN: 0813817439; 2001.

### Ramped Dosing Regimens

Any of the dosing regimens disclosed herein, e.g., any of the dosing regimens disclosed in the “Exemplary Dosing Regimens” section above, may contain a ramping period. “Ramp” or “ramping period” as the terms are used herein refer to a portion of a dosing regimen over which the amount of antibody or fragment administered increases from a ramp day at the beginning of the ramping period to a ramp day at the end of the ramping period. “Ramp day” as the term is used herein refers to a given day within the ramping period. In certain embodiments, the ramping period is at least two days, e.g., at least three days, at least four days, at least five days, at least six days, at least seven days, at least eight days, at least nine days, at least ten days, at least eleven days, at least twelve days, at least thirteen days, at least fourteen days, or more. In certain embodiments, the ramping period is at most fourteen days, e.g., at most thirteen days, at most twelve days, at most eleven days, at most ten days, at most nine days, at most eight days, at most seven days, at most six days, at most five days, at most four days, at most three days, or fewer. In certain embodiments, the ramping period is two days, three days, four days, five days, six days, seven days, eight days, nine days, ten days, eleven days, twelve days, thirteen days, fourteen days or more. In certain embodiments, the ramping period is four days.

In certain embodiments, the ramping period is two days, three days, four days, five days, six days, seven days, eight days, nine days, ten days, eleven days, twelve days, thirteen days, fourteen days or more. In certain embodiments, the ramping period is four days. The ramp (or ramping period) does not include the first day of two or more days in which the dose of anti-CD3 antibody or antigen binding fragment administered is the same or decreases. In this case, the two or more days are non-ramp days and the non-ramp period consists of the two or more days. Thus, for example, in a regimen consisting of an anti-CD3 antibody or antigen binding fragment dosing schedule of 0.1 mg on day 1, 0.2 mg on day 2, 0.3 mg on day 3, 0.4 mg on day 0.4, 0.5 mg on day 6, 0.5 mg on day 7, and 0.5 mg on day 8, days 1-4 are ramp days, the ramp (or ramp period) consists of days 1-4, days 5-8 are non-ramp days, and the non-ramp period consists of days 5-7. It is understood that one or more ramps (ramp periods) can follow one or more non-ramp periods. The first day of a ramp is a day on which a dose that is administered is less than the immediately following dose. A pre-ramp day is a day prior to the first day of a ramp. Naturally, a pre-ramp day can be one or more days (e.g., 2 or more, 3 or more, 4 or more, 5 or more, 6 or more, 7, or more, 8 or more, nine or more, 10 or more, 11 or more, 12 or more, 13 or more, or 14 or more) before the first day of a ramp.

Methods disclosed herein that include a ramping period permit administration of higher cumulative doses of the anti-CD3 antibody or antigen binding antibody fragment with decreased pro-inflammatory cytokine release and immunogenicity, and with minimal to no perturbation of Epstein Barr Virus immunity. In certain embodiments, methods disclosed herein that include a ramping period facilitate higher individual doses later in a dosing regimen than would be possible with traditional dosing regimens.

In general a ramping period comprises the following characteristics: the anti-CD3 antibody or antigen binding fragment thereof is administered in an amount greater than about 0.1 mg and less than about 0.5 mg on ramp day one; the amount of the antibody or fragment administered on ramp day two is less than about 0.5 mg greater than the amount of the antibody or fragment administered on ramp day one; the amount of the antibody or fragment administered on ramp day three is less than about 0.55 mg greater than the amount of the antibody or fragment administered on ramp day two; the amount of the antibody or fragment administered on ramp day four is less than about 0.6 mg greater than the amount of the antibody or fragment administered on ramp day three; the amount of the antibody or fragment administered on ramp day four is more than 0.3 mg greater than the amount of the antibody or fragment administered on ramp day one; and the amount of the antibody or fragment administered at least one ramp day is at least about 0.5 mg.

In certain embodiments, an anti-CD3 antibody or antigen binding fragment thereof is administered in an amount greater than about 0.1 mg and less than about 0.5 mg on ramp day one. For example, the anti-CD3 antibody or fragment may be administered in an amount of about 0.1 mg, 0.15 mg, 0.2 mg, 0.25 mg, 0.3 mg, 0.35 mg, 0.4 mg, 0.45 mg, or 0.5 mg on ramp day one.

In certain embodiments, the amount of anti-CD3 antibody or antigen binding fragment thereof administered increases between ramp day one and ramp day four of a given dosing regimen. In certain embodiments, the amount of increase between ramp day one and ramp day four is more than about 0.3 mg. For example, the amount of the anti-CD3 antibody or fragment administered may increase more than about 0.3 mg, more than about 0.35 mg, more than about 0.4 mg, more than about 0.45 mg, more than about 0.5 mg, more than about 0.55 mg, more than about 0.6 mg, more than about 0.65 mg, more than about 0.7 mg, more than about 0.75 mg, more than about 0.8 mg, more than about 0.85 mg, more than about 0.9 mg, more than about 0.95 mg, more than about 1.0 mg, more than about 1.1 mg, more than about 1.2 mg, more than about 1.3 mg, more than about 1.4 mg, more than about 1.5 mg, more than about 1.6 mg, more than about 1.7 mg, more than about 1.8 mg, more than about 1.9 mg, more than about 2 mg, more than about 2.5 mg, more than about 3 mg, more than about 3.5 mg, more than about 4 mg, more than about 4.5 mg, more than about 5 mg, or more.

In certain embodiments, the amount of anti-CD3 antibody or antigen binding fragment thereof administered increases on each day between ramp day one and ramp day four of a given dosing regimen such that the total increase between ramp day one and ramp day four is more than about 0.3 mg. In certain embodiments, the amount of the anti-CD3 antibody or fragment administered between ramp day one and ramp day four of a given dosing regimen increases by more than about 0.3 mg, but the amount of anti-CD3 antibody or fragment administered does not increase on each day. For example, the amount of the anti-CD3 antibody or fragment administered may remain constant or even decrease between, e.g., ramp day one and ramp day two, ramp day two and ramp day three, or ramp day three and ramp day four, but the total amount nevertheless increases by more than about 0.3 mg between ramp day one and ramp day four.

In certain embodiments, the amount of anti-CD3 antibody or antigen binding fragment thereof administered on ramp day two of a given dosing regimen is less than about 0.5 mg greater than the amount of the anti-CD3 antibody or fragment administered on ramp day one of the dosing regimen. For example, the amount of the anti-CD3 antibody or fragment administered on ramp day two of the dosing regimen may be less than about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on ramp day one. In certain embodiments, the amount of the anti-CD3 antibody or fragment administered on ramp day two of the dosing regimen is about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on ramp day one. In certain embodiments, the amount of the anti-CD3 antibody or fragment administered on ramp day two of the dosing regimen is about equal to the amount administered on ramp day one. In certain embodiments, the amount of the anti-CD3 antibody or fragment administered on ramp day two of the dosing regimen is less than the amount administered on ramp day one. For example, the amount of the anti-CD3 antibody or fragment administered on ramp day two of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on ramp day one. In certain embodiments, the amount of the anti-CD3 antibody or fragment administered on ramp day two of the dosing regimen is more than about 0.5 mg less than the amount administered on ramp day one.

In certain embodiments, the amount of the anti-CD3 antibody or antigen binding fragment thereof administered on ramp day three of a given dosing regimen is less than about 0.55 mg greater than the amount of the anti-CD3 antibody or fragment administered on ramp day two of the dosing regimen. For example, the amount of the anti-CD3 antibody or fragment administered on ramp day three of the dosing regimen may be less than about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on ramp day two. In certain embodiments, the amount of the anti-CD3 antibody or fragment administered on ramp day three of the dosing regimen is about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on ramp day two. In certain embodiments, the amount of the anti-CD3 antibody or fragment administered on ramp day three of the dosing regimen is about equal to the amount administered on ramp day two. For example, the amount of the anti-CD3 antibody or fragment administered on ramp day three of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on ramp day two. In certain embodiments, the amount of the anti-CD3 antibody or fragment administered on ramp day three of the dosing regimen is more than about 0.5 mg less than the amount administered on ramp day two.

In certain embodiments, the amount of anti-CD3 antibody or antigen binding fragment thereof administered on ramp day four of a given dosing regimen is less than about 0.6 mg greater than the amount of the anti-CD3 antibody or fragment administered on ramp day three of the dosing regimen. For example, the amount of the anti-CD3 antibody or fragment administered on ramp day four of the dosing regimen may be less than about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on ramp day three. In certain embodiments, the amount of the anti-CD3 antibody or fragment administered on ramp day four of the dosing regimen is about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on ramp day three. In certain embodiments, the amount of the anti-CD3 antibody or fragment administered on ramp day four of the dosing regimen is about equal to the amount administered on ramp day three. In certain embodiments, the amount of the anti-CD3 antibody or fragment administered on ramp day four of the dosing regimen is less than the amount administered on ramp day three. For example, the amount of the anti-CD3 antibody or fragment administered on ramp day four of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on ramp day three. In certain embodiments, the amount of the anti-CD3 antibody or fragment administered on ramp day four of the dosing regimen is more than about 0.5 mg less than the amount administered on ramp day three.

In certain embodiments, the amount of anti-CD3 antibody or antigen binding fragment thereof administered on ramp day four of a given dosing regimen is at least about 0.5 mg. For example, the amount of the anti-CD3 antibody or fragment administered on ramp day four of a given dosing regimen can be at least about 0.5 mg, at least about 0.55 mg, at least about 0.6 mg, at least about 0.65 mg, at least about 0.7 mg, at least about 0.75 mg, at least about 0.8 mg, at least about 0.85 mg, at least about 0.9 mg, at least about 0.95 mg, at least about 1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2 mg, at least about 2.5 mg, at least about 3 mg, at least about 3.5 mg, at least about 4 mg, at least about 4.5 mg, at least about 5 mg, or higher. In certain embodiments, the amount of the anti-CD3 antibody or fragment administered on ramp day four of a given dosing regimen is about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.2 mg, about 1.3 mg, about 1.4 mg, about 1.5 mg, about 1.6 mg, about 1.7 mg, about 1.8 mg, about 1.9 mg, about 2 mg, about 2.5 mg, about 3 mg, about 3.5 mg, about 4 mg, about 4.5 mg, about 5 mg, or higher.

In certain embodiments, an anti-CD3 antibody or antigen binding fragment thereof is administered on at least one pre-ramp day prior to ramp day one. For example, the anti-CD3antibody or fragment may be administered on one, two, three, four, five, six, seven, eight, nine, ten, or more pre-ramp days prior to ramp day one. In certain embodiments, the amount of the anti-CD3 antibody or fragment administered on at least one pre-ramp day does not exceed 0.3 mg, e.g., does not exceed 0.25 mg, 0.2 mg, 0.15 mg, 0.1 mg, 0.05 mg, or less. In certain embodiments, the amount of the anti-CD3 antibody or fragment administered on at least one pre-ramp day is about 0.1 mg. In certain embodiments, the amount of the anti-CD3 antibody or fragment administered on at least one pre-ramp day is about 0.2 mg. In certain embodiments, the amount of the anti-CD3 antibody or fragment administered on at least one pre-ramp day is about 0.3 mg.

### Dosing Regimens Based on Body Weight and Body Surface Area

In certain embodiments, anti-CD3 antibodies or antigen binding fragments thereof can be administered without regard to body weight of the patient or to the body surface area of the patient. For example, any of the dosing regimens described above can be administered to patient regardless of weight or body surface area.

In certain embodiments, anti-CD3 antibodies or antigen binding fragments thereof can be administered based on the body weight of the patient. Such body weight-based dosing regimens can be useful when, for example, the subject is significantly overweight or underweight compared to a typical patient. Such body weight-based dosing regimens can also be useful when the subject is a juvenile and thus weighs significantly less than a typical adult patient. In certain embodiments, by calibrating the amount of the anti-CD3 antibody or fragment administered based on the body weight of the patient, a more uniform amount of antibody or fragment thereof can be achieved across patients who differ in body weight.

Any dosing regimen, such as one of those described in the present specification, can be administered based on the body weight of the patient. A typical adult human has a body weight of between 70 and 80 kg, and dosing regimens described herein can be calculated on a per weight basis based on, for example, either of these weights. For example, if a non-weight-based dosing regimen calls for 0.1 mg of anti-CD3 antibody or antigen binding fragment thereof to be administered on particular day, a weight-based dose can be administered in an amount equal to 1.25 μg/kg (based on an 80 kg person in the original dosing regimen), or in an amount equal to 1.43 μg/kg (based on a 70 kg person in the original dosing regimen). Other daily doses of the anti-CD3 antibody or fragment for a given dosing regimen can be similarly calculated. Thus, in certain embodiments, an adult with a higher body weight can receive a greater amount of the anti-CD3 antibody or fragment, while adults with a lower body weight can receive a smaller amount of the anti-CD3 antibody or fragment.

As another non-limiting example, the dosing regimen disclosed in Example 5 can be administered on the basis of a patient's weight. Example 5 discloses the following dosing schedule: 0.1 mg on day 1, 0.3 mg on day 2, 0.5 mg on day 3, 0.9 mg on day 4, and 1.3 mg on day 5. Based on a typical 80 kg patient, for example, one can administer the dosing schedule of Example 5 to a patient based on his or her specific body weight as follows: 1.25 μg/kg on day 1, 3.75 μg/kg on day 2, 6.25 μg/kg on day 3, 11.25 μg/kg on day 4, and 16.25 μg/kg on day 5. Based on a typical 70 kg patient, for example, one can administer the dosing schedule of Example 5 to a patient based on his or her specific body weight as follows: 1.43 μg/kg on day 1, 4.29 μg/kg on day 2, 7.15 μg/kg on day 3, 12.87 μg/kg on day 4, and 18.59 μg/kg on day 5. Those of ordinary skill in the art can calculate the amounts to be given based on any given body weight for any of the dosing regimens disclosed herein.

Juveniles have a significantly lower body weight than that of the typical adult. For example, a juvenile patient may have a body weight of 40 kg. In such a case, again taking a non-weight-based dosing regimen that calls for 0.1 mg of anti-CD3 antibody or fragment, the juvenile patient may be administered 50 μg (based on 1.25 μg/kg for an 80 kg adult) or 57.2 μg (based on 1.43 μg/kg for a 70 kg adult) of an anti-CD3 antibody or fragment.

In certain embodiments, anti-CD3 antibodies or antigen binding fragments thereof can be administered based on the body surface area of the patient. Such body surface-based dosing regimens can be useful when, for example, the subject is significantly larger or smaller compared to a typical patient. Such body surface-based dosing regimens can also be useful when the subject is a juvenile whose body surface is significantly smaller than that of a typical adult patient. In certain embodiments, by calibrating the amount of antibody or fragment administered based on the surface area of the patient, a more uniform amount of antibody or fragment can be achieved across patients who differ in body surface areas.

Any dosing regimen, such as one of those described in the present specification, can be administered based on the body surface area of the patient. A typical adult human has a body surface area of approximately 1.7 square meters, and dosing regimens described herein can be calculated based on such a body surface area. For example, if a dosing regimen that is not based on body surface area calls for 0.1 mg of the anti-CD3 antibody or antigen binding fragment thereof to be administered on particular day, a body surface-based dose can be administered in an amount equal to 58.82 μg/square meter (based on an average adult body surface of 1.7 square meters). Other daily doses of the anti-CD3 antibody or fragment for a given dosing regimen can be similarly calculated. Thus, in certain embodiments, an adult with a larger body surface area can receive a greater amount of the anti-CD3 antibody or fragment, while adults with a smaller body surface area can receive a smaller amount of the anti-CD3 antibody or fragment.

As another non-limiting example, the dosing regimen disclosed in Example 5 can be administered on the basis of a patient's body surface area. Example 5 discloses the following dosing schedule: 0.2 mg on day 1, 0.4 mg on day 2, 0.6 mg on day 3, 0.8 mg on day 4, and 1.1 mg on day 5. Based on an average body surface area of 1.7 square meters per patient, for example, one can administer the dosing schedule of Example 5 to a patient based on his or her specific body surface area as follows: 117.65 μg/square meter on day 1, 235.29 μg/kg on day 2, 352.94 μg/kg on day 3, 470.59 μg/kg on day 4, and 647.06 μg/kg on day 5. Those of ordinary skill in the art can calculate the amounts to be given based on any given body surface area for any of the dosing regimens disclosed herein.

Juveniles have a lower body surface area than that of the typical adult. For example, a juvenile patient may have a body surface area of 1.3 square meters. In such a case, again taking a dosing regimen not based on body surface area that calls for 0.2 mg of anti-CD3 antibody or antigen binding fragment thereof, the juvenile patient may be administered 153.85 μg of the anti-CD3 antibody or fragment.

Those of ordinary skill in the art will be able to calculate weight-based and body surface-based dosing regimens that correspond to any of the variety of dosing regimens disclosed in the present specification, and will be able to administer such dosing regimens to a patient.

Dosing Regimens Based on Molecular Weight of Antibody or Fragment

In certain embodiments, anti-CD3 antibodies or antigen binding fragments thereof can be administered without regard to the molecular weight of the anti-CD3 antibody or fragment, or to the number of antigen binding sites in a given anti-CD3 antibody or fragment. For example, any of the dosing regimens described above can be administered to patient regardless of molecular weight or number of antigen binding sites.

“Molecular weight” is a term and concept well known to those of ordinary skill in the art. The molecular weight of a compound or composition is the weight of one molecule of the compound or composition, relative to the unified atomic mass unit u (defined as 1/12 the mass of one molecule of the carbon-12 isotope). A compound or composition having a given molecular weight can also be quantified by molar mass, which has a numerical value that is the average molecular weights of the molecules in the compound or composition multiplied by Avogadro's constant (approximately 6.022×10²³). Molar mass is expressed in terms of grams per mole.

Antibodies vary in molecular weight based on, for example, the length and amino acid composition of the heavy and light chain polypeptide sequences that make up the protein part of the antibody. Moreover, as is known to those of ordinary skill in the art, the molecular weight of an antibody varies according to the extent of post-translational modification the antibody undergoes. For example, antibodies are often subjected to glycosylation, in which one or more carbohydrate moieties is covalently attached to either the heavy or light chain polypeptide sequence. Even amongst a population of antibodies with identical heavy and light chain polypeptide sequences, the extent of glycosylation can vary. The molecular weights of many antibodies are known in the art. Additionally, the molecular weight of a particular antibody can be empirically determined with any of a variety of tools known to those of ordinary skill in the art such as, without limitation, mass spectrometry. Determining the molecular weight of any particular antibody is within the abilities of those of ordinary skill in the art.

Antigen binding antibody fragments also vary in molecular weight based on, for example, the length and amino acid composition of the heavy and light chain polypeptide sequences and post-translational glycosylation patterns. Certain antigen binding antibody fragments, such as without limitation, Fab fragments, F(ab′)₂ fragments, and scFv fragments, are typically of a much lower molecular weight that that of an antibody that includes both heavy and light polypeptide chains. As with full-length antibodies, the molecular weight of particular antibody fragment can be empirically determined with any of a variety of tools known to those of ordinary skill in the art such as, without limitation, mass spectrometry, and is within the abilities of those of ordinary skill in the art

In certain embodiments, anti-CD3 antibodies or antigen binding fragments thereof can be administered based on the molecular weight of that antibody or fragment. Such molecular weight-based dosing regimens can be useful when, for example, a practitioner desires to administer a dosing regimen of a particular anti-CD3 antibody or fragment, the molecular weight of which differs from the molecular weight of another anti-CD3 antibody or fragment used in an identical or similar dosing regimen. In certain embodiments, by calibrating the amount of the anti-CD3 antibody or fragment administered based on the molecular weight of the particular anti-CD3 antibody or fragment, a more uniform molar amount of the anti-CD3 antibody or fragment can be administered to a patient.

For example, otelixizumab has an average molecular weight of approximately 145 kDa. Thus, if a particular dosing regimen calls for 0.1 mg of anti-CD3 antibody to be administered to a patient on a particular day, the patient can be administered approximately 6.90×10⁻¹⁰ moles of otelixizumab. Doses of different anti-CD3 antibodies or antigen binding fragments thereof can be similarly calculated based on the molecular weight of those antibodies or fragments thereof. In certain embodiments, the anti-CD3 antibody or fragment with a larger molecular weight is administered to the patient in a greater per-weight amount. In other embodiments, an anti-CD3 antibody or fragment with a smaller molecular weight is administered to the patient in a lower per-weight amount.

As another non-limiting example, the dosing regimen disclosed in Example 5 can be administered based on the molecular weight of the anti-CD3 antibody or antigen binding fragment thereof to be administered. Example 5 discloses the following dosing schedule: 0.2 mg on day 1, 0.4 mg on day 2, 0.6 mg on day 3, 0.8 mg on day 4, and 1.1 mg on day 5. Based on a reference antibody with a molecular weight of 145 kDa, for example, one can administer the dosing schedule of Example 5 to a patient based on the specific molecular weight of the antibody of fragment to be administered as follows: 1.38×10⁻⁹ moles on day 1, 2.76×10⁻⁹ moles on day 2, 4.14×10⁻⁹ moles on day 3, 5.52×10⁻⁹ moles on day 4, and 7.59×10⁻⁹ moles on day 5. Those of ordinary skill in the art can calculate the molar amounts of the anti-CD3 antibody or fragment to be given for any of the dosing regimens disclosed herein.

In certain embodiments, anti-CD3 antibodies or antigen binding fragments thereof can be administered based on the number of antigen binding sites present on the anti-CD3 antibody or fragment. As is known to those of ordinary skill in the art, a whole antibody includes two distinct antigen binding sites which are located in the hypervariable regions of the antibody. The antigen binding sites of whole antibodies are formed by an interaction between the variable regions of the heavy and light chains. Each antigen binding site is capable of binding one antigen. Thus, whole antibodies are capable of binding two antigens. Certain antibody fragments can also include two antigen binding sites. For example, a F(ab′)₂ fragment lacks the constant region of a whole antibody, yet retains two antigen binding sites. Certain antibody fragments include only a single antigen binding site. For example, Fab fragments and scFv fragments lack the constant region of a whole antibody, and include only a single antigen binding site. Those of ordinary skill in the art will be aware of various antibody fragments, and will know how many antigen binding sites each fragment contains.

In certain embodiments, anti-CD3 antibodies or antigen binding fragments thereof can be administered based on the number of antigen binding sites present in a given anti-CD3 antibody or fragment. Such antigen binding site-based dosing regimens can be useful when, for example, a practitioner desires to administer a dosing regimen of a particular anti-CD3 antibody or fragment that includes a different number of antigen binding sites as compared to the number of antigen binding sites of another anti-CD3 antibody or fragment used in an identical or similar dosing regimen. In certain embodiments, by calibrating the amount of anti-CD3 antibody or fragment administered during a dosing regimen based on the number of antigen binding sites that the anti-CD3 antibody or fragment possesses, a more uniform number of antigen binding sites can be administered to a patient.

For example, otelixizumab possesses two antigen binding sites per molecule. Thus, if a particular dosing regimen calls for 0.1 mg of antibody to be administered to a patient on a particular day, the patient can be administered approximately 0.1 mg of otelixizumab, or 0.2 mg of an anti-CD3 antibody or fragment that possesses only one antigen binding site per molecule. Doses of different anti-CD3 antibodies or fragments can be similarly calculated based on the number of antigen binding sites those antibodies or fragments possess. In certain embodiments, an anti-CD3 antibody or fragment with one antigen binding site per molecule is administered to the patient in a greater amount than an anti-CD3 antibody or fragment with two or more antigen binding sites per molecule. In other embodiments, an anti-CD3 antibody or fragment with two or more antigen binding sites per molecule is administered to the patient in a lower amount than an anti-CD3 antibody or fragment with only one antigen binding site per molecule.

As another non-limiting example, the dosing regimen disclosed in Example 5 can be administered based on the number of antigen binding site the anti-CD3 antibody or fragment to be administered possesses. Example 5 discloses the following dosing schedule: 0.2 mg on day 1, 0.4 mg on day 2, 0.6 mg on day 3, 0.8 mg on day 4, and 1.1 mg on day 5. Based on a reference antibody having two antigen binding sites, for example, one can administer an anti-CD3 antibody or fragment having only one antigen binding site to a patient according to the dosing schedule as follows: 0.4 mg on day 1, 0.8 mg on day 2, 1.2 mg on day 3, 1.6 mg on day 4, and 2.2 mg on day 5. Those of ordinary skill in the art can calculate the amount of anti-CD3 antibody or fragment to be given for any of the dosing regimens disclosed herein based on the number of antigen binding sites the anti-CD3 antibody or fragment possesses.

Those of ordinary skill in the art will be able to calculate weight-based and body surface-based dosing regimens that correspond to any of the variety of dosing regimens disclosed in the present specification, and will be able to administer such dosing regimens to a patient.

Moreover, those of ordinary skill in the art will be able to choose a dosing regimen of a particular anti-CD3 antibody or antigen binding fragment thereof based on a combination of one or more of: the body weight of a patient, the body surface area of a patient, the molecular weight of the antibody or fragment, and the number of antigen binding sites of the antibody or fragment. For example, a patient that weighs more than 80 kg can be administered an anti-CD3 antibody or fragment that possesses only one antigen binding site. In such an example, a larger amount of an anti-CD3 antibody or fragment can be administered to account for (1) the patient's increased weight, and (2) the fact that the anti-CD3 antibody or fragment has fewer antigen binding sites than a bivalent whole antibody. Upon reading the present specification, those of ordinary skill in the art will be able to administer an anti-CD3 antibody or fragment to a patient in a dosing regimen specifically tailored to the physical characteristics of the patient and/or the molecular properties of the anti-CD3 antibody or fragment.

### PK/PD Parameters

The presently disclosed methods are not limited in any way by any particular mechanism of action. Nevertheless, a number of pharmacodynamic (PD) effects of treating T cells with reduced Fc (gamma) receptor-binding anti-CD3 antibodies or CD3-binding fragments thereof, according to methods disclosed herein, are observable. For convenience these reduced Fc (gamma) receptor-binding anti-CD3 antibodies and CD3-binding fragments are sometimes referred to in this PK/PD Parameters section as “CD3-binding agents.”

In broad terms, the immunoregulatory effects seen after administration of CD3-binding agents can be divided into two phases that can overlap to some degree. Thus in the initial early phase (from an hour up to about 14 days) following exposure of T cells (CD4+ and CD8+) to such CD3-binding agents (in vivo and in vitro), immunoregulatory effects that occur include down-modulation of CD3/TCR complexes on the surfaces of the T cells, induction of T cell anergy or hyporesponsiveness to antigen, induction of apoptosis of T cells, and a decrease in the numbers of T cells (CD4+ T cells and CD8+ T cells). With respect to in vitro exposures, solid or gel substrate (e.g., tissue culture well bottom or agarose bead)-bound anti-CD3 antibodies, and CD3-binding fragments thereof, that have reduced ability to bind Fc (gamma) receptors do not qualify as “CD3-binding agents” (as defined above) in this substrate-bound form, since they act in the same way as anti-CD3 antibodies with normal, wild-type Fc (gamma) receptor binding activity in the presence of Fc (gamma) receptor expressing cells. In the later phase (from one day to 16 weeks or more) after the exposure, the levels of immunosuppressive CD4+ T cells (Tregs) expressing both cell surface CD25 (i.e., CD25+) and the FoxP3 transcription factor (FoxP3+) are found to increase. Notably, no increase in CD8+, CD25+, FoxP3+ cells is seen. Some or all of these events are interrelated.

T cells that undergo apoptosis as a result of exposure to CD3-binding agents, which is generally by the Fas/Fas ligand pathway, are those that are activated by antigen prior to the exposure (and are progressing through the cell cycle) and are not resting T cells. T cells in the S-G2 phase of the cell cycle are particularly sensitive to this type of apoptosis. The decreases in the numbers of CD4+ and CD8+ T cells that are seen in the first phase appear to reflect retrafficking of T cells (e.g., from the blood to lymphoid tissue and/or target organs) and, to a relatively small extent, the above-described apoptosis.

The initial decrease of antigen responsiveness of T cells that have not undergone apoptosis is to some degree correlated with CD3/TCR down-modulation on the surface of the T cells. Nevertheless, there are conditions under which drastically reduced antigen responsiveness in the T cells is observed in the face of significant levels of cell surface TCR (see, e.g., Schwartz (2003) Annu Rev. Immunol. 21:305-334, the disclosure of which is incorporated herein by reference in its entirety). These findings indicate that, while antigen hyporesponsiveness in the T cells exposed to CD3/TCR-binding agents is due at least in part to down-modulation of CD3/TCR complexes, it is likely also due to the other effects such as active CD3/TCR-mediated anergy induction. It is also clear that, while transient exposure of T cells to lower doses of CD3-binding agents results in transient anergy or antigen hyporesponsiveness of T cells and cell-surface CD3/TCR down-modulation (with full recovery within less than 24 hours of exposure), longer exposure to somewhat higher doses results in much longer, if not permanent, anergy or antigen hyporesponsiveness (see, e.g., Anasetti et al. (1990) J. Exp. Med. 172:1691-1700; and Forman et al. (2009) Immune Privilege and Tolerance-Therapeutic Antibody Approaches. In: Recombinant Antibodies for Immunotherapy, M. Little, Ed., Cambridge University Press, pp. 350-369, the disclosures of which are incorporated herein by reference in their entirety). Down-modulation of CD3/TCR in response to CD3-binding agents seems to be largely due to internalization of CD3-binding agent:CD3/TCR complexes rather than masking of the CD3/TCR complex by the binding agent.

The transient effects (anergy or antigen hyporesponsiveness of T cells and cell-surface CD3/TCR down-modulation) indicated above to occur as a result of exposure to CD3-binding agents are seen even when repeated doses (e.g., on a daily basis) are administered. The anergy/antigen hyporesponsiveness and cell-surface CD3/TCR down-modulation occur after the first administration but the levels of both return to normal (i.e., the levels prior to the first administration) by the time of the second administration. The same effect is seen after all subsequent administrations unless much higher doses are administered and/or the cells are exposed to the CD3-binding agent for a much longer time. This pattern of decrease and increase in these parameters is referred to herein as a “saw tooth pattern.” Interestingly, with respect to the levels of both CD4+ and CD8+ T cells, while a saw tooth pattern is seen, it is accompanied by an overall decrease in the total numbers of the cells during the course of the CD3-binding agent (see, e.g., Examples 2-4). Thus, after each successive administration, the rebound seen after the initial decrease in cell numbers after an administration is to a lower level than after the immediately previous administration.

It is likely that the induction of anergy or antigen hyporesponsiveness in T cells by these CD3-binding agents, which, as indicated above have reduced ability to bind to Fcγ receptors, is analogous to that of altered peptide ligands (APL) (see, e.g.: Sloan-Lancaster et al. (1993) Nature 363:156-159; Sloan-Lancaster et al. (1994) Cell 79:913-922; and Madrenas et al. (1995) 267:515-518, the disclosures of which are incorporated herein by reference in their entirety), TCR binding of which results in weak or incomplete activation of T cells. One likely mechanism of CD3-binding agent-induced anergy induction involves reduction in the relative proportion of cell surface CD3/TCR multimeric clusters to cell-surface monovalent CD3/TCR complexes. It has been shown that CD3/TCR complexes on T cells occur as both monovalent units and as multivalent clusters, the latter existing in a wide range of multiplicities (from two to greater than 20 CD3/TCR monomers), and the monomer in each case containing a TCR aα and β chain (or a TCR γ and δ chain), one CD3 δ, two CD3ε, one CD3 γ, and two CD3 ζ chains (see, e.g.: Alarcón et al. (2006) EMBO Reports 7: 490-495; and Schamel et al. (2005) J. Exp. Med. 202(4): 493-503, the disclosures of which are incorporated herein by reference in their entirety). Thus, by exposing T cells to increasing concentrations of CD3-binding agents, the relative level of higher avidity CD3/TCR multimer clusters is decreased, leaving behind the lower avidity CD3/TCR monovalent units and thereby reducing the potential CD3/TCR signal strength and T cell responsiveness. The lower the level of multimers left after exposure, the longer it will take a particular T cell to recover fully activating signal strength responsiveness by synthesizing new multimers and/or converting monomeric units into multivalent complexes. This phenomenon could also explain the “conditioning” effect observed when an animal (e.g., a human) is administered a dosing regimen that includes a ramping period, as disclosed herein. Without wishing to be bound by theory, it is hypothesized that conditioning may result from the lower ramping doses being sufficient to modulate but not activate, so that when subsequent larger activating doses are given later in a dosing regimen, the signal strength is weak or incomplete, leading to relative low responses and anergy. At some critical concentration of CD3-binding agent and/or length of exposure of the T cell to the CD3-binding agent, the T cell will be rendered anergic for an extremely long time, possibly for its lifetime. The relative susceptibility of T cells to anergy induction would depend on a number of factors, including the relative number of multimeric CD3/TCR clusters to monovalent CD3/TCR units and the relative number of monomeric units in the clusters.

The induction of CD4+ Tregs that occurs later in the response of CD4+ T cells to CD3-binding agents is likely to be relatively more important in the long-term beneficial effects of CD3-binding agents to immune-related (especially T cell-mediated) diseases, including autoimmune diseases such as type I diabetes (insulin-dependent diabetes mellitus (IDDM)), psoriasis, multiple sclerosis, and rheumatoid arthritis. Their induction very likely involves factors (e.g., transforming growth factor β (TGF-β)) produced by, and/or cell-cell interactions with, the hyporesponsive (or completely anergized) T cells described above, as well as antigen presenting cells such as dendritic cells, and does not necessarily require contacting of the Treg precursor cells themselves with a CD3-binding agent.

In light of the above considerations, this document provides methods for treating a human with an anti-CD3 antibody or an antigen binding fragment thereof comprising: administering the antibody or the fragment to the human in a regimen such that: (a) in a therapy window of at least two days and no more than 6 days, for at least 48 hours of the window, the mean level of free CD3/TCR complexes on CD4+ and on CD8+ T cells is at least 10 percent and less than 40 percent of the mean baseline level; (b) in a therapy window of 7 days or more, for at least 48 hours of the first 6 days of the window, the mean level of free CD3/TCR complexes on CD4+ and on CD8+ T cells is at least 10 percent and less than 40 percent of the mean baseline level; (c) in a therapy window of at least 8 days, for at least 48 hours of the window, the mean level of free CD3/TCR complexes on CD4+ and on CD8+ T cells is at least 10 percent and less than 40 percent of the mean baseline level and at least 30 of the 48 hours occur after the first 6 days of the window; or in a therapy window of at least 4 days, for at least 90 hours of the window, the mean level of free CD3/TCR complexes on CD4+ and on CD8+ T cells is at least 10 percent and less than 40 percent of the mean baseline level, wherein the antibody or fragment does not bind, or has reduced binding, to at least one class of Fc (gamma) receptor as compared to the OKT3 antibody.

Also provided are methods for treating a human with an anti-CD3 antibody or an antigen binding fragment thereof comprising: administering the antibody or the fragment to the human in a regimen such that: (a) in a therapy window of at least two days and no more than 6 days, for at least 12 hours of the window, the mean level of free CD3/TCR complexes on CD4+ and on CD8+ T cells is at least 20 percent and less than 30 percent of the mean baseline level; (b) in a therapy window of 7 days or more, for at least 18 hours of the first 6 days of the window, the mean level of free CD3/TCR complexes on CD4+ and on CD8+ T cells is at least 20 percent and less than 30 percent of the mean baseline level; (c) in a therapy window of at least 7 days, for at least 24 hours of the window, the mean level of free CD3/TCR complexes on CD4+ and on CD8+ T cells is at least 20 percent and less than 30 percent of the mean baseline level and at least 15 of the at least 24 hours occur after the first 6 days of the window; or (d) in a therapy window of at least 7 days, for at least 40 hours of the window, the mean level of free CD3/TCR complexes on CD4+ and on CD8+ T cells is at least 20 percent and less than 30 percent of the mean baseline level and at least half of the at least 40 hours in the window occur after the first 6 days of the window, wherein the antibody or fragment does not bind, or has reduced binding, to at least one class of Fc (gamma) receptor as compared to the OKT3 antibody.

This document also provides methods for treating a human with an anti-CD3 antibody or an antigen binding fragment thereof, the method comprising: administering the antibody or the fragment to the human in a regimen such that: (a) in a regimen of 3 days or more, the daily dose administered is at least 1 mg and no greater than 3 mg in any 24 hour period and on each of at least 3 days of the regimen; (b) in a regimen of 3 days or more, the daily dose administered is at least 1 mg and no greater than 1.75 mg in any 24 hour period and on each of at least 3 days of the regimen; (c) in a regimen of 3 days or more, the daily dose administered is at least 14 μg/kg and no greater than 42 μg/kg in any 24 hour period and on each of at least 3 days of the regimen; (d) in a regimen of 3 days or more, the total dose administered is 2.5 mg to 9 mg and no greater than 3 mg on any single day of the regimen; (e) in a regimen of 3 days or more, the total dose administered is 2.5 mg to 6.6 mg and no greater than 2.2 mg on any single day of the regimen; (f) in a regimen of 3 days or more, the total dose administered is 35 μg/kg to 126 μg/kg and no greater than 42 μg/kg on any single day of the regimen; (g) in a regimen of 3 days or more, the total dose administered is 35 micrograms/kg to 93 μg/kg and no greater than 31 μg/kg on any single day of the regimen; (h) in a therapy window of at least three days, where a dose is administered over a period of 24 hours or more, the total dose administered to the human is at least 2.5 mg; or (i) in a therapy window of at least three days, where a dose is administered over a period of 24 hours or more, the total dose administered to the human is at least 35 μg/kg, wherein the antibody or fragment does not bind, or has reduced binding, to at least one class of Fc (gamma) receptor as compared to the OKT3 antibody and, optionally, the three days are not continuous.

Also provided are methods of treating a human with an anti-CD3 antibody, or an antigen binding fragment thereof, the method comprising administering the antibody or fragment to the human in a regimen that comprises a dosing ramp of at least four days, wherein the antibody or fragment does not bind or has reduced binding to at least one class of the Fc (gamma) receptor as compared to the OKT3 antibody.

The therapy windows and/or regimens in any of the above methods can be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, or more. The length of the therapy window can be the same as the length of the regimen or longer.

In any of the above methods, the first at least 1 day, at least 2 days, at least 3 days, at least 4 days, at least 5 days, at least 6 days, at least 7 days, at least 8 days, at least 9 days, at least 10 days, or more of the regimen are a dosing ramp. Preferably, the first at least 4 days of the regimen are a dosing ramp.

The mean free level of CD3/TCR complexes in the therapy windows of any of the above methods for treating a human with an anti-CD3 antibody or an antigen binding fragment thereof is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40% of the mean baseline level of free CD3/TCR complexes. Preferably, the mean free level of CD3/TCR complexes in the therapy windows of any of the above methods for treating a human with an anti-CD3 antibody or an antigen binding fragment thereof is at least 10%. Preferably, the mean free level of CD3/TCR complexes in the therapy windows of any of the above methods for treating a human with an anti-CD3 antibody or an antigen binding fragment thereof is no more than about 40%, about 45%, about 50%, about 55%, about 60%, about 65%, about 70%, about 75%, or about 80%. Preferably, the mean free level of CD3/TCR complexes in the therapy windows of any of the above methods for treating a human with an anti-CD3 antibody or an antigen binding fragment thereof is at least 10% and no more than about 40%, at least 15% and no more than 35%, or at least 20% and no more than 30%.

The mean free level of CD3/TCR complexes in the therapy windows of any of the above embodiments can be continuous or not continuous.

In any of the above methods, the maximum daily dose of the anti-CD3 antibody or fragment is 10 mg or less, 9.5 mg or less, 9 mg or less, 8.5 mg or less, 8 mg or less, 7.5 mg or less, 7 mg or less, 6.5 mg or less, 6 mg or less, 5.5 mg or less, 5 mg or less, 4.5 mg or less, 4 mg or less, 3.5 mg or less, 3 mg or less, 2.5 mg or less, 2 mg or less, or 1.5 mg or less, or 1 mg or less. In a preferred embodiment, the maximum daily dose of the anti-CD3 antibody or fragment is 3 mg or less, 2 mg or less, 1.75 mg or less, or 1.5 mg or less.

In any of the above methods, at least one dose of the anti-CD3 antibody is greater than about 0.1 mg, 0.2 mg, 0.3 mg, 0.4 mg, or 0.5 mg, or more.

In any of the above methods, in a regimen of 3 days or more, the daily dose of anti-CD3 antibody or fragment thereof administered is at least 0.5 mg, 1.0 mg, 1.5 mg, 2.0 mg, 2.5 mg, 3 mg, 3.5 mg, 4 mg, 4.5 mg or 5.0 mg.

In any of the above methods, in a regimen of 3 days or more, the total dose of anti-CD3 antibody or fragment thereof administered is at least 0.5 mg, 1.0 mg, 1.5 mg, 2.0 mg, 2.5 mg, 3 mg, 3.5 mg, 4 mg, 4.5 mg, 5.0 mg, 5.5 mg, 6.0 mg, 6.5 mg, 7.0 mg, 8.0 mg, 8.5 mg, 9.0 mg, 9.5 mg, 10 mg, or more.

In any of the above methods, in a regimen or therapy window of 1 day or more, 2 days or more, 3 days or more, 4 days or more, or 5 days or more, the total dose of anti-CD3 antibody or fragment thereof administered is at least 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 105 μg/kg, 110 μg/kg, 115 μg/kg, 120 μg/kg, 125 μg/kg, 130 μg/kg, 135 μg/kg, 140 μg/kg, 145 μg/kg, 150 μg/kg, or more. Preferably, in a regimen or therapy window of at least three days, the dose administered over a period of 24 hours or more is at least 35 μg/kg.

In any of the above methods, for at least days two to four of the ramp, the dosing produces a daily decrease in the mean maximum levels of free CD3/TCR complexes on CD4+ and on CD8+ T cells as compared to the mean baseline levels, wherein the differences between the mean maximum levels on any day of the at least day two to day four of the ramp and the mean maximum levels on the preceding day are not greater than about 5%, 10%, 15%, 20%, 25%, 30%, 35%, 40%, 50%, 55%, 60%, 65%, 70%, 80%, or more of the mean maximum levels on the preceding day. Preferably, the mean maximum levels of free CD3/TCR complexes on CD4+ and on CD8+ T cells as compared to the mean baseline levels on the preceding day are not greater than about 20% to about 30%, and more preferably no greater than about 25% of the mean maximum levels on the preceding day.

In any of the above methods, for at least days two to four of the ramp, the dosing produces a daily decrease in mean maximum levels of free TCR complex molecules on CD4+ and on CD8+ T cells as compared to the mean baseline levels, wherein the differences between the mean maximum levels on any day of the at least day two to day four of the ramp and the mean maximum levels on the preceding day are at least 1%, 2%, 3%, 4%, 5%, 6%, 7%, 8%, 9%, 10%, or more of the mean maximum levels on the preceding day.

In any of the above methods, for at least days two through four of the ramp, the dosing of the ramp produces a daily increase in minimum concentration of the anti-CD3 antibody or the fragment (Cₘᵢₙ) in the peripheral blood or peripheral blood plasma of the human. Preferably, the first dose of the ramp produces a Cmin in the peripheral blood of the human of no greater than about 0.005 mg/L, 0.01 mg/L, 0.02 mg/L, 0.03 mg/L, 0.04 mg/L, 0.05 mg/L, 0.6 mg/L, 0.07 mg/L, 0.08 mg/L, 0.09 mg/L, 0.10 mg/L, 0.5 mg/L, or 1.0 mg/L.

In any of the above methods, the dosing produces a daily increase in Cₘᵢₙ in the peripheral blood of the human of at least 1%, 2%, 3%, 4%, 5%, 6%, 7%, 8%, 9%, 10%, 11%, 12%, 13%, 14%, 15%, or more as compared to the Cₘᵢₙ in the peripheral blood of the human on the preceding day.

As a specific example, the anti-CD3 antibody or fragment can be administered over a dosing regimen of at least five days; wherein the antibody or fragment is administered on day one, and wherein the amount of antibody or fragment administered on each of days one and two does not exceed 0.5 mg per day; wherein the amount of antibody or fragment administered on day three is less than about 0.5 mg greater than the amount of antibody or fragment administered on day two; wherein the amount of antibody or fragment administered on day four is less than about 0.55 mg greater than the amount of antibody or fragment administered on day three; wherein the amount of antibody or fragment administered on day five is less than about 0.6 mg greater than the amount of antibody or fragment administered on day four; wherein the amount of antibody or fragment administered on day five is more than 0.3 mg greater than the amount of antibody or fragment administered on day two; and wherein the amount of antibody or fragment administered on day five is at least about 0.5 mg.

In any of the above methods, the method can cause modulation in the activity or numbers of one or both of antigen-specific effector (Teff) or antigen-specific regulatory (Treg) T cells. In some cases, the number of antigen-specific T regulatory cells can be enhanced.

In any of the above methods, on at least one day of the treatment window, the mean levels of CD3/TCR complexes on CD4+ and on CD8+ T-cells can be decreased by at least 5%, 10%, 15%, 20%, 25%, 30% or more and less than 100%, 99% 98%, 97%, 96%, 95%, 94%, 93%, 92%, 91%, 90%, 89%, 88%, 87%, 86%, 85%, or less, as compared to the mean baseline levels.

In any of the above methods, one or more pre-ramp doses are administered prior to dose day one. Also, in any of the above methods, the ramp can be given prior to the administration of a maximum daily dose such that the ramp causes a reduction in one or both of the (a) production of at least one pro-inflammatory cytokine or tryptase and (b) immunogenicity, as compared to one or both of the (i) production of the at least one pro-inflammatory cytokine or tryptase and (ii) immunogenicity that is observed after administration of the maximum dose without a ramp of at least four days. Pro-inflammatory cytokines include without limitation, IL2, IL6, IL10, IFN-gamma, and TNF-alpha.

In any of the above methods, the dosing regimen can be a follows, (a) the amount of antibody or fragment administered on day one is about 0.1 mg, 0.2 mg, 0.3 mg, 0.4 mg, 0.5 mg, 0.6 mg, 0.7 mg, 0.8 mg, 0.9 mg, 1.0 mg, or more; the amount of antibody or fragment administered on day two is about 0.1 mg, 0.2 mg, 0.3 mg, 0.4 mg, 0.5 mg, 0.6 mg, 0.7 mg, 0.8 mg, 0.9 mg, 1.0 mg, or more; the amount of antibody or fragment administered on day three is about 0.1 mg, 0.2 mg, 0.3 mg, 0.4 mg, 0.5 mg, 0.6 mg, 0.7 mg, 0.8 mg, 0.9 mg, 1.0 mg, or more; the amount of antibody or fragment administered on day four is about 0.1 mg, 0.2 mg, 0.3 mg, 0.4 mg, 0.5 mg, 0.6 mg, 0.7 mg, 0.8 mg, 0.9 mg, 1.0 mg, or more; the amount of antibody or fragment administered on day five is about 0.1 mg, 0.2 mg, 0.3 mg, 0.4 mg, 0.5 mg, 0.6 mg, 0.7 mg, 0.8 mg, 0.9 mg, 1.0 mg, 1.25 mg, 1.5 mg, 1.75 mg, 2.0 mg or more; the amount of antibody or fragment administered on day six is about 0.1 mg, 0.2 mg, 0.3 mg, 0.4 mg, 0.5 mg, 0.6 mg, 0.7 mg, 0.8 mg, 0.9 mg, 1.0 mg, 1.25 mg, 1.5 mg, 1.75 mg, 2.0 mg or more; the amount of antibody or fragment administered on day seven is about 0.1 mg, 0.2 mg, 0.3 mg, 0.4 mg, 0.5 mg, 0.6 mg, 0.7 mg, 0.8 mg, 0.9 mg, 1.0 mg, 1.25 mg, 1.5 mg, 1.75 mg, 2.0 mg or more; and the amount of antibody or fragment administered on day eight is about 0.1 mg, 0.2 mg, 0.3 mg, 0.4 mg, 0.5 mg, 0.6 mg, 0.7 mg, 0.8 mg, 0.9 mg, 1.0 mg, 1.25 mg, 1.5 mg, 1.75 mg, 2.0 mg or more.

In a specific embodiment, the dosing regimen can be a follows, (a) the amount of antibody or fragment administered on day one is about 0.1 mg, the amount of antibody or fragment administered on day two is about 0.2 mg; the amount of antibody or fragment administered on day three is about 0.3 mg; the amount of antibody or fragment administered on day four is about 0.75 mg; the amount of antibody or fragment administered on day five is about 1.0 mg; the amount of antibody or fragment administered on day six is about 1.25 mg; the amount of antibody or fragment administered on day seven is about 1.5 mg; and the amount of antibody or fragment administered on day eight is about 1.75 mg; and (b) the amount of antibody or fragment administered on day one can be about 0.2 mg; the amount of antibody or fragment administered on day two is about 0.4 mg; the amount of antibody or fragment administered on day three is about 0.6 mg; the amount of antibody or fragment administered on day four is about 0.8 mg; and the amount of antibody or fragment administered on day five is about 1.1 mg.

In any of the above embodiments, the antibody or fragment has a binding affinity constant of at least 0.05, 0.1, 0.2, 0.3, 0.4, 0.5, 0.6, 0.7, 0.8, 0.9, 0.95, 1.0 μg/mL or more, and a kₑₗ of about 0.5, 0.6, 0.7, 0.8, 0.9, 1.0, 1.1, 1.2, 1.3, 1.4, 1.5, 1.6, 1.7, 1.8, 1.9, 2.0 or more per day.

In any of the above methods, the anti-CD3 antibody or fragment has an IC₅₀ of less than about 150, 125, 100, 75, 50, 25 ng/ml or less. Preferably, the anti-CD3 antibody or fragment has an IC₅₀ of about 75 ng/ml.

In any of the above embodiments, the antibody has a half-life of between 1 and 50, between 2 and 40, between 3 and 30, between 5 and 20, or between 10 and 15 hours at the doses administered in the regimen. Preferably, the anti-CD3 antibody or fragment has a half-life of between 5 and 20 hours at the doses administered in the regimen.

Also provided are methods of inducing: hyporesponsiveness and/or anergy and/or apoptosis; decreases in the numbers of CD4+ and CD8+ T cells; cell surface CD3/TCR down-modulation, and down-modulation of the relative level of multivalent CD3/TCR clusters (as compared to monovalent CD3/TCR units) in target T cells (e.g., CD4+ and CD8+ T cells to which the CD3-binding agents bind). Also provided are methods for inducing CD4+, CD25+, FoxP3+ Treg cells. The latter methods can result in a higher net increase in the level of antigen-specific Treg cells as compared to the net increase in antigen-specific T effector cells (Teff). This can be achieved by exposing the T cells in vivo or in vitro for lengths of time and/or concentrations of the CD3-binding agents that result in a higher net increase in the level of antigen-specific Treg cells than the net increase antigen-specific Teff cells. Thus, in cases where the level of Treg increases, the level of Teff can increase less, decrease, or not increase or decrease. In cases where the level of Treg decreases, the level of Teff decreases more. Where the level of Treg is not changed, the level of Teff decreases. All the above methods involve exposing target T cells to CD3-binding agents either in vivo or in vitro. Where the exposing is in vitro, the CD3-binding agents are in solution rather than bound to a solid or gel substrate (see above). In the induction of Treg cells, the precursor of the Treg can be, but is not necessarily, a target T cell (as the term is used above). Moreover, CD3-binding agents can bind to established CD4+CD25+FoxP3+ Tregs and thereby enhance their suppressive activity. The dosing and scheduling regimens and methods of administration for performing in vivo exposures can be any of those disclosed herein, as are the subjects to which the methods can be applied.

While the target T cells are more commonly CD4+ T cells, it is understood that they can also be CD8+ T cells. Moreover, CD4+ and CD8+ effector T cells (e.g., pathogenic T cells involved in a disease process) are subject to the suppressive activity of CD4+CD25+FoxP3+ Tregs. However, it is understood that CD25+, FoxP3+ T regs per se are CD4+ and not CD8+. The CD3/TCR down-modulation can be complete (100%) or partial (e.g., at least or not greater than: 10%:20%; 30%; 40%; 50%; 60%; 70%; 80%; 90%; 95%; or 98%). The down-modulation of the number of multivalent CD3/TCR clusters (i.e., units containing more than one CD3/TCR complex unit (see above)) can be similarly complete or partial. An anergic T cell is one that has substantially no responsiveness (i.e., less than 5%) as compared to the responsiveness that the T cell would have had without exposure to a CD3-binding agent or the average responsiveness of T cells having the same CD4/CD8 cell surface marker as well as other markers known in the art to be associated with pre-exposure, or lack thereof, to antigen. T cells can be naive T cells (i.e., those never pre-exposed to antigen), activated T cells (i.e., T cells exposed to antigen and displaying any of a variety of T cell activities, e.g., proliferation, cytotoxic activity, and/or cytokine production), or memory T cells (i.e., T cells previously exposed to antigen and having an enhanced ability to respond to the same antigen and not necessarily displaying an activated cell phenotype). Cell surface markers positively (+) and negatively (−) associated with naive T cells include: CD45RA+, CD26L+, CD45 edited isoforms (CD45RB, CD45RC, CD45RAB, CD45RAC, CD45RBC, CD45RO, CD45R (ABC))−, CD25−, CD44−, and CD69−. Cell surface markers positively (+) associated with activated T cells include: CD25+, CD69+, HLA-DR+, CD38+, and GITR+. Memory T cells fall into three broad categories, which are categorized as follows: central memory T cells (memory stem cells) (T_{CM}) (L-selectin+, chemokine receptor CCR7+, and produce interleukin (IL)-2 (IL-2) but not IL-4 or interferon γ (IFN-γ)); effector memory T cells (T_{EM}) and closely related effector memory T cells RA (T_{EMRA}) (L-selectin−, CCR7−, and produce IL-4 and IFN-γ).

With respect to pharmacokinetic (PK) data, it has been possible to determine PK parameters for a CD3-binding agent of interest (the TRX4 antibody, also known as otelixizumab) using data collected from a number of clinical studies (see Table 1). The serum otelixizumab concentrations versus time were described by a one-compartment model with Michaelis-Menten (MM) saturable elimination:

$$\frac{d {C}_{p}}{d t} = Input / {V}_{d} - {V}_{max} {C}_{p} / ( {K}_{m} + {C}_{p} )$$

$${C}_{p} ( 0 ) = 0$$

where Cₚ is serum concentration of otelixizumab, V_{d} is the volume of distribution, Vₘₐₓ is the capacity of the elimination process, and Kₘ is the affinity constant or the serum otelixizumab concentration at which the elimination rate attains one-half of Vₘₐₓ.

TABLE 1
Clinical Studies of Otelixizumab Included in PK Analysis

| | | | | Number |
|---|---|---|---|---|
| | Group | | | of |
| Study | or Cohort | Doses (mg)^{a} | Disease^{b} | subjects |
| I | Group A | 24, 8, 8, 8, 8, 8 | D | 3 |
| | Group B | 8, 8, 8, 8, 8, 8 | D | 37 |
| II | Cohort 1 | 1 | P | 4 |
| | Cohort 2 | 2 | P | 4 |
| | Cohort 3 | 4 | P | 8 |
| III | Cohort 1 | 0.1, 0.1, 0.1 | D | 4 |
| | Cohort 2 | 0.5, 0.5, 0.5 | D | 3 |
| | Cohort 9 | 0.1, 0.3, 0.5 | D | 4 |
| | Cohort 10 | 0.3, 0.5, 1.0 | D | 1 |
| | Cohort A | 0.1, 0.2, 0.3, 0.5 | D | 4 |
| | Cohort A | 0.05, 0.1, 0.15, 0.25 | D | 1 |
| | (½) | | | |
| | Cohort B | 0.1, 0.2, 0.3, 0.75 | D | 4 |
| | Cohort C | 0.1, 0.2, 0.3, 1.0 | D | 1 |
| | CH1 | 0.1, 0.2, 0.3, 0.5 × 5 | D | 16 |
| | CH2 | 0.1, 0.2, 0.3, 0.75 × 5 | D | 18 |
| | CH3 | 0.1, 0.2, 0.3, 0.75, 1, 1.25, 1.5, | D | 6 |
| | | 1.75 | | |

^{a}Doses were given once daily for 1 to 8 days.
^{b}D—Type 1 diabetes; P—Psoriasis.

In Study I (Table 1), otelixizumab was administered 6 times. In Group A, otelixizumab concentrations remained more or less constant over the 6 days of dosing, whereas in group B they increased slightly, suggesting accumulation of the drug.

In Study II (Table 1), otelixizumab was administered only once. Extensive sampling was done over the 24 hours after drug administration. For the 1 mg and 2 mg doses, the concentrations decreased to below the LLQ (lower limit of quantification) in about 0.2 day. For the 4 mg dose, concentrations above LLQ were observed up to 0.8 day. A few subjects showed a biphasic decline with a very rapid first phase.

In Study III (Table 1), otelixizumab was administered daily for up to 8 days. Doses were substantially lower than in Studies I and II, and as a result, most (83%) concentrations were below the LLQ. Due to the limited amount of available PK data, simultaneous analysis of the PK and PD (pharmacodynamic) data was necessary to recover PK profiles. The model building process started with linear PK; however, the individual empirical Bayesian estimates of volume of distribution were dose-dependent, suggesting nonlinearity. Thus, MM elimination was used, leading to substantial improvement in the model. Such kinetic parameters were estimated Kₘ=0.968 μg/mL and Vₘₐₓ=1.35 μg/mL/day. At low concentrations, such as those observed in Study III, otelixizumab was eliminated linearly with elimination rate constant kₑₗ=Vₘₐₓ/Kₘ=1.39 day⁻¹. At high concentrations, elimination was saturated. The V_{d} was estimated as 13.9 L with between-subject variability of about 76%.

Biphasic elimination from serum is usually observed after an intravenous dose of intact antibodies. The intact antibodies rapidly distribute primarily to the highly perfused organs such as kidney, lung and liver. The volume of distribution often equals the plasma volume, 2-3 L. For otelixizumab, the V_{d} of 13.9 L was determined assuming a one-compartment model with MM elimination. This value of V_{d} suggests antibody distribution outside the blood or occurrence of nonspecific binding. Antigen binding can significantly affect the PK of a mAb. Target-mediated drug disposition models were proposed and successfully applied to describe the PK of certain mAbs. In the case of otelixizumab, elimination by binding to CD3/TCR complexes did not affect its PK. After otelixizumab administration, the CD3/TCR is down-modulated from T cell surfaces, and the transient trafficking and re-distribution of lymphocytes reduces the total pool of receptors available for binding. The MM elimination was used to approximate observed nonlinearities. The affinity constant (Kₘ=0.968 μg/mL) suggests that PK may become nonlinear at high concentrations such as those observed in Study I. For the dose ranges used in Study III, and to some degree in Study II, the drug is eliminated under linear conditions with a kₑₗ of 1.39 day⁻¹ and a corresponding half-life of 0.50 day. Intact human IgG₁ exhibits a long half-life of about 3 weeks due to the catabolic protection and recycling by the neonatal Fc receptor (FcRn). For otelixizumab the half-life is much shorter, suggesting that this protection pathway is not active, likely due to the single amino acid substitution in the Fc region which eliminates the only glycosylation site and alters the spatial configuration of the Fc region.

In view of the above PK considerations, in certain embodiments, the present disclosure provides a CD3-binding agent (see above) and a pharmaceutical composition containing it. The CD3-binding agent is an antibody (or CD3-binding fragment thereof) that binds to human CD3 with an affinity constant (Kₘ) of at least 0.968 μg/mL and can have a kₑₗ of about 1.39 day⁻¹. Moreover, its half life can be about 0.50 day when administered to a human.

The CD3-binding agent can show non-linear PK at high concentrations (about 8 mg to about 48 mg per day) and linear PK at low concentrations (about 0.1 to about 21 mg per day). Other features of the CD3-binding agent can be those described herein for otelixizumab (TRX4). Moreover the CD3-binding agent can be used in any of the methods and subjects described herein.

In certain embodiments, a pathogenic effect observed in the animal (e.g., on day five) or later of the dosing regimen is decreased or eliminated compared to the pathogenic effect that would be observed that day if the animal were administered a different dosing regimen. “Pathogenic effect”, as the term is used herein, refers to any adverse effect that results directly or indirectly from a given dosing regimen. A pathogenic effect may be, for example, increased cytokine release, (Epstein Barr Virus) EBV activation, or immunogenicity. In certain embodiments, the different dosing regimen lacks a ramping period. In certain embodiments, the different dosing regimen comprises a dose higher than 0.5 mg on either day one or day two of the different dosing regimen.

In certain embodiments, dosing regimens disclosed herein result in a reduced level of release of at least one cytokine compared to an animal that is administered an equivalent dosing regimen of an anti-CD3 antibody or CD3-binding fragment thereof that does not exhibit reduced binding to the Fc (gamma) receptor. For example the release of the at least one cytokine may be reduced by at least 50%, e.g., at least 55%, at least 60%, at least 65%, at least 70%, at least 75%, at least 80%, at least 85%, at least 90%, at least 95%, at least 96%, at least 97%, at least 98%, at least 99%, or more. In certain embodiments, such a cytokine may be a pro-inflammatory cytokine including, but not limited to, IL2, IL6, IL10, IFN-gamma, (tumor necrosis factor) TNF-alpha, and/or tryptase. Those of ordinary skill in the art will be aware of other pro-inflammatory cytokines, and will be able to measure their levels in a subject that has been administered any of the dosing regimens disclosed herein.

In certain embodiments, dosing regimens disclosed herein optimize modulation of antigen-specific and regulatory T cells, thereby allowing a shorter course of therapy (optionally, with controlled dose escalation) with a better tolerated regimen. As used herein, “modulation of a T cell” can mean affecting the activity of the T cell (e.g., activated or anergic), the levels of CD3/TCR complex expressed on the T cell surface, and/or the total numbers of T cells.

In some embodiments, optimized dose escalation parameters as disclosed herein can improve tolerability of a dosing regimen with controlled escalation of anti-CD3 antibody or antigen binding fragment thereof over at least the first five days of the regimen. Such parameters can achieve higher PK/PD parameters sooner in the regimen and can reduce pro-inflammatory cytokine release and immunogenicity, as compared to dosing at such levels with insufficient escalation to condition for the next dose administered.

In certain embodiments, for, e.g., certain immune-related diseases such as, e.g., rheumatoid arthritis, dosing regimens can be designed to specifically enhance the opportunity for modulation of antigen-specific and regulatory T cells. Such dosing regimens optimize the duration of therapy in a preferred window to achieve enhanced numbers of T regulatory cells, based on differential susceptibility of T regulatory and T effector cells to anti-CD3-mediated inhibition of T cell activation during the initial early phase (from an hour up to about 14 days) following exposure of T cells (CD4+ and CD8+), as described in detail, above.

### Immune-Related Diseases

In certain embodiments, methods disclosed herein can be used to treat a subject (e.g., a human patient) suffering from an immune-related disease. “Immune-related disease”, as the term is used herein, refers to a disease that is associated with at least one abnormal immune phenomenon. For example, one class of immune-related diseases comprises autoimmune diseases. An autoimmune disease typically results when the subject's immune system is activated against one or more components (cells, tissues, or cell/tissue-free molecules) of the subject and attacks that subject's s own organs, tissues or cells, instead of attacking, for example, foreign bacteria, viruses and other infectious agents or cancer cells. Every mammalian subject exhibits autoimmunity to some extent, but such autoimmunity normally does not result in a disease state since the immune system regulates and suppresses normal autoimmunity. Autoimmune diseases develop when there is a disruption in the immune system's regulation. Autoimmune diseases can also result when there is a molecular alteration in a subject's cell that is recognized by the immune system, such that the immune system recognizes the altered cell as “foreign.”

Another example of an immune-related disease is a disease associated with the effects of organ, tissue, or cell transplantation. Cells transplanted into a subject rarely exhibit the same antigens on their surfaces as the subject's endogenous cells. Thus, a transplant subject's immune system often attacks and rejects the foreign cells. Certain immunosuppressive drugs are typically used to abrogate or decrease this immune attack, but such drugs often cause undesirable side effects, including for example, the risk of developing opportunistic infections as a result of decreased immune responses. In severe cases, an immune system attack on a transplanted organ can lead to organ failure or more serious systemic complications, such as, for example, graft-versus-host disease (GVHD) where the graft (e.g., bone marrow) includes immune-system effector cells (e.g., effector T cells) or precursors thereof.

Exemplary immune-related diseases include, but are not limited to, adrenergic drug resistance, alopecia areata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, dense deposit disease, diseases associated with effects from organ transplantation, discoid lupus, essential mixed cryoglobulinemia, fibromyalgia-fibromyositis, glomerulonephritis (e.g., IgA nephrophathy), gluten-sensitive enteropathy, Goodpasture's syndrome, graft vs. host disease (GVHD), Graves' disease (including e.g., Graves thyroiditis and Graves opthalmopathy), Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, Insulin Resistance Syndrome, juvenile arthritis, lichen planus, lupus erythematosus, Meniere's disease, Metabolic Syndrome, mixed connective tissue disease, multiple sclerosis, Myasthenia Gravis, myocarditis, diabetes (e.g., Type I diabetes or Type II diabetes), neuritis, other endocrine gland failure, pemphigus vulgaris, pernicious anemia, polyarteritis nodosa, polychrondritis, Polyendocrinopathies, polyglandular syndromes, polymyalgia rheumatica, polymyositis and dermatomyositis, post-MI, primary agammaglobulinemia, primary biliary cirrhosis, psoriasis, psoriatic arthritis, Raynauld's phenomenon, relapsing polychondritis, Reiter's syndrome, rheumatic heart disease, rheumatoid arthritis, sarcoidosis, scleroderma, Sjogren's syndrome, stiff-man syndrome, systemic lupus erythematosus, takayasu arteritis, temporal arteritis/giant cell arteritis, ulcerative colitis, urticaria, uveitis, Uveitis Opthalmia, vasculitides such as dermatitis herpetiformis vasculitis, vitiligo, and Wegener's granulomatosis.

### Anti-CD3 Antibodies and Antigen Binding Fragments Thereof

Any of a variety of anti-CD3 antibodies or antigen binding fragments thereof can be employed in the dosing regimens described herein. In certain embodiments, the antibody or fragment is a human antibody or fragment. In certain embodiments, the anti-CD3 antibody or fragment is a non-human antibody or fragment, e.g., a mouse or rat antibody or fragment. In certain embodiments, the anti-CD3 antibody or fragment is chimeric in that it contains human heavy and/or light chain constant regions. In certain embodiments, the anti-CD3 antibody or fragment is humanized in that it contains one or more human framework regions in the variable region together with non-human (e.g., mouse, rat, or hamster) complementarity-determining regions (CDRs) of the heavy and/or light chain. In certain embodiments, the anti-CD3 antibody is monoclonal. In certain embodiments, the fragment is derived from a monoclonal antibody (e.g., cleaved at its hinge region to generate a F(ab′)₂ fragment). In certain embodiments, the anti-CD3 antibody is a polyclonal antibody population in that it comprises a plurality of different antibodies, each of which binds to the same antigen. In certain embodiments, the fragment is derived from a polyclonal anti-CD3 antibody population.

In certain embodiments, an antibody fragment is a Fab fragment, a F(ab′)₂ fragment, a scFv fragment, a diabody, a linear antibody, a multispecific antibody fragment such as a bispecific, a trispecific, or a multispecific antibody (e.g., a diabody, a triabody, a tetrabody), a minibody, a chelating recombinant antibody, a tribody or bibody, an intrabody, a nanobody, a small modular immunopharmaceutical (SMIP), a binding-domain immunoglobulin fusion protein, a camelid antibody, or a V_{HH} containing antibody.

In certain embodiments, an anti-CD3 antibody or antigen binding fragment thereof to be employed in one or more of the dosing regimens disclosed herein binds a human CD3 polypeptide. A variety of anti-human CD3 antibodies and fragments are known in the art. Such anti-CD3 antibodies and fragments are useful, for example, when the animal to be treated is a human. In certain embodiments, an anti-CD3 antibody or fragment to be employed in one or more of the dosing regimens disclosed herein binds a non-human CD3. For example, a non-human mammal may be administered an anti-CD3 antibody or fragment, which antibody or fragment binds a CD3 present in that animal. Any of a variety of non-human mammals are known, and can be administered an anti-CD3 antibody or fragment that binds a CD3 present in such that animal. Non-limiting examples include dogs, cats, cows, horses, sheep, goats, pigs, mice, rats, and hamsters. The anti-CD3 antibodies and fragments can be of the same species or a different species. Moreover, they can be analogous to the chimeric and humanized antibodies described herein. Thus, when treating a horse, for example, the CD3 antibody can contain heavy and/or light chain variable regions of another species (e.g., mouse, rat, hamster, or human) and horse heavy and/or light chain constant regions (chimeric heavy and/or light chains). Alternatively, heavy and/or light chains can contain all the CDRs from another species (as above) with the rest of the heavy and/or light chain being horse (horse analogs of humanized heavy and light chains). Moreover, the heavy chain or the light chain can be of the chimeric type and the other chain can be of the horse analog of the humanized chain. The same principles apply to anti-CD3 antibodies and fragments for use in any of the exemplary species listed above.

In certain embodiments, an anti-CD3 antibody or antigen binding fragment thereof to be employed in one or more of the dosing regimens disclosed herein binds a CD3 epsilon polypeptide, e.g., a human CD3 epsilon polypeptide. In certain embodiments, the anti-CD3 antibody or fragment to be employed in one or more of the dosing regimens disclosed herein binds a CD3 gamma polypeptide, e.g., a human CD3 gamma polypeptide. In certain embodiments, the anti-CD3 antibody or fragment to be employed in one or more of the dosing regimens disclosed herein binds a CD3 delta polypeptide, e.g., a human CD3 delta polypeptide. In certain embodiments, the anti-CD3 antibody or fragment to be employed in one or more of the dosing regimens disclosed herein binds a CD3 zeta polypeptide, e.g., a human CD3 zeta polypeptide.

In certain embodiments, an antibody to be employed in one or more of the dosing regimens disclosed herein is otelixizumab, a humanized aglycosylated antibody. Otelixizumab, also known as TRX4, comprises a heavy chain having the sequence set forth in SEQ ID NO: 1 EVQLLESGGGLVQPGGSLRLSCAASGFTFSSFPMAWVRQAPGKGLEWVSTISTSGGRTY YRDSVKGRFTISRDNSKNTLYLQMNSLRAEDTAVYYCAKFRQYSGGFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYASTYRVVSVLTVLHQDWLNGKEYKCKVSNKALPAPIEKTISKAKGQPREPQVYTLP PSRDELTKNQVSLTCLVKGFYPSDIAVEWESNGQPENNYKTTPPVLDSDGSFFLYSKLTV DKSRWQQGNVFSCSVMHEALHNHYTQKSLSLSPGK], and a light chain having the sequence set forth in SEQ ID NO: 2 [DIQLTQPNSVSTSLGSTVKLSCTLSS GNIENNYVHWYQLYEGRSPTTMIYDDDKRPDGVPDRFSGSIDRSSNSAFLTIHNVAIEDE AIYFCHSYVSSFNVFGGGTKLTVLRQPKAAPSVTLFPPSSEELQANKATLVCLISDFYPGA VTVAWKADSSPVKAGVETTTPSKQSNNKYAASSYLSLTPEQWKSHRSYSCQVTHEGST VEKTVAPTECS]. In certain embodiments, the anti-CD3 antibody or fragment to be employed in one or more of the dosing regimens disclosed herein comprises the heavy chain variable region of otelixizumab, as set forth in SEQ ID NO: 3 [EVQLLESGGGLVQPGGS LRLSCAASGFTFSSFPMAWVRQAPGKGLEWVSTISTSGGRTYYRDSVKGRFTISRDNSK NTLYLQMNSLRAEDTAVYYCAKFRQYSGGFDYWGQGTLVTVSS]. In certain embodiments, the anti-CD3 antibody or fragment to be employed in one or more of the dosing regimens disclosed herein comprises the light chain variable region of otelixizumab, as set forth in SEQ ID NO: 4 [DIQLTQPNSVSTSLGSTVKLSCTLSSGNIENNYVHWYQLYEG RSPTTMIYDDDKRPDGVPDRFSGSIDRSSNSAFLTIHNVAIEDEAIYFCHSYVSSFNVFGG GTKLTVLR].

In certain embodiments, an anti-CD3 antibody or antigen binding fragment thereof to be employed in one or more of the dosing regimens disclosed herein comprises one or more complementarity determining regions (CDRs) of otelixizumab. For example, an antibody or fragment may include one or more of the following: the otelixizumab heavy chain variable complementarity determining region 1 (VH CDR1) comprising the amino acid sequence as set forth in SEQ ID NO: 5 [SFPMA], the otelixizumab heavy chain variable complementarity determining region 2 (VH CDR2) comprising the amino acid sequence as set forth in SEQ ID NO: 6 [TISTSGGRTYYRDSVKG], the otelixizumab heavy chain variable complementarity determining region 3 (VH CDR3) comprising the amino acid sequence as set forth in SEQ ID NO: 7 [FRQYSGGFDY], the otelixizumab light chain variable complementarity determining region 1 (VL CDR1) comprising the amino acid sequence as set forth in SEQ ID NO: 8 [TLSSGNIENNYVH], the otelixizumab light chain variable complementarity determining region 2 (VL CDR2) comprising the amino acid sequence as set forth in SEQ ID NO: 9 [DDDKRPD], or the otelixizumab light chain variable complementarity determining region 3 (VL CDR3) comprising the amino acid sequence as set forth in SEQ ID NO: 10 [HSYVSSFNV]. In certain embodiments, the antibody or fragment comprises each of the complementarity determining regions comprising the amino acid sequences set forth in SEQ ID NOs: 5-10.

The anti-CD3 antibody or antigen binding fragment thereof for use in the methods described herein can contain any combination of light and heavy chain, any combination of light and heavy chain variable regions, and any combination of light and heavy chain CDRs described above.

In certain embodiments, an anti-CD3 antibody or antigen binding fragment thereof to be employed in one or more of the dosing regimens disclosed herein exhibits reduced binding to at least one class of Fc (gamma) receptor. In certain embodiments, binding of the modified anti-CD3 antibody or fragment to at least one Fc (gamma) receptor is reduced as compared to the binding exhibited by the OKT3 antibody. OKT3 is a mouse antibody that is well-known to those of ordinary skill in the art. OKT3 binds the CD3 antigen, and is available from a variety of commercial sources (e.g., eBioscience™ at www.ebioscience.com). Additionally, a hybridoma cell line expressing the OKT3 antibody has been deposited under ATCC number CRL-8001. In certain embodiments the anti-CD3 antibody or fragment to be employed in one or more of the dosing regimens disclosed herein exhibits at least 25% reduced binding to at least one class of Fc (gamma) receptor as compared to the binding that would be observed with the OKT3 antibody. For example, the anti-CD3 antibody or fragment may exhibit at least 25%, 30%, 35%, 40%, 45%, 50%, 55%, 60%, 65%, 70%, 75%, 80%, 85%, 90%, 95%, 96%, 97%, 98%, 99% or more reduced binding.

In certain embodiments, binding of the modified anti-CD3 antibody or antigen binding fragment thereof to at least one class of Fc (gamma) receptor is reduced as compared to the binding exhibited by the huOKT3-gamma-1 and/or huOKT3-gamma-1(A318) antibodies as described in Xu et al., Cellular Immunology, 200, 16-26 (2000), incorporated herein by reference in its entirety. In certain embodiments the anti-CD3 antibody or fragment to be employed in one or more of the dosing regimens disclosed herein exhibits at least 25% reduced binding to at least one class of Fc (gamma) receptor as compared to the binding that would be observed with the huOKT3-gamma-1 and/or huOKT3-gamma-1(A318) antibodies. For example, the anti-CD3 antibody or fragment may exhibit at least 25%, 30%, 35%, 40%, 45%, 50%, 55%, 60%, 65%, 70%, 75%, 80%, 85%, 90%, 95%, 96%, 97%, 98%, 99% or more reduced binding.

In certain embodiments, binding of the modified antibody or antigen binding fragment thereof to at least one class of Fc (gamma) receptor is reduced as compared to the binding exhibited by the IgG1 immunoglobulin molecule produced by the ARH-77 cell line deposited under ATCC catalog number CRL-1621. In certain embodiments the anti-CD3 antibody or fragment to be employed in one or more of the dosing regimens disclosed herein exhibits at least 25% reduced binding to at least one class of Fc (gamma) receptor as compared to the binding that would be observed with the IgG1 antibody produced by the ARH-77 cell line deposited under ATCC catalog number CRL-1621. For example, the antibody or fragment may exhibit at least 25%, 30%, 35%, 40%, 45%, 50%, 55%, 60%, 65%, 70%, 75%, 80%, 85%, 90%, 95%, 96%, 97%, 98%, 99% or more reduced binding.

In certain embodiments, an anti-CD3 antibody or antigen binding fragment thereof to be employed in one or more of the dosing regimens disclosed herein does not bind (e.g., exhibits no detectable binding) to at least one class of Fc (gamma) receptor.

In certain embodiments, an anti-CD3 antibody or antigen binding fragment thereof that exhibits reduced binding to at least one class of Fc (gamma) receptor comprises a modification that results in the reduced binding. In certain embodiments, such an anti-CD3 antibody or fragment may be modified at one or more amino acid residues within a heavy chain, a light chain, or both. The glycosylation state of an antibody or fragment may affect its binding to one or more classes of Fc (gamma) receptor. In certain embodiments, glycosylation of an anti-CD3 antibody or fragment is altered by modifying one or more amino acid residues within a heavy chain, a light chain, or both. For example, otelixizumab comprises a human IgG1 heavy chain constant region that has been modified by replacing an asparagine at position 299 of SEQ ID NO: 1 with an alanine This modification results in decreased glycosylation of the antibody and significantly decreased binding of the antibody to major Fc receptors compared to antibody molecules having wild type IgG1 constant regions, leading to decreased pro-inflammatory cytokine release and immunogenicity, and no perturbation of Epstein Barr Virus immunity. In certain embodiments, an anti-CD3 antibody or fragment comprises an alanine at an amino acid position corresponding to amino acid position 299 of SEQ ID NO: 1. Position 299 of SEQ ID NO: 1 corresponds to amino acid residue number 297 of IgG heavy chains, according to the Kabat canonical numbering system (see Kabat E A, Wu T T, Perry H, Gottesman K, and Foeller C. (1991) Sequences of Proteins of Immunological Interest, Fifth Edition. NIH Publication No. 91-3242, incorporated herein by reference in its entirety.) All IgG molecules contain a single conserved N-linked glycosylation site in each of their Cγ2 domains, which conserved glycosylation site corresponds to amino acid residue number 297 of IgG heavy chains, according to the Kabat canonical numbering system (see Arnold et al., The Impact of Glycosylation on the Biological Function and Structure of Human Immunoglobulins, Annu Rev. Immunol. 2007. 25:21-50, 2007, incorporated herein by reference in its entirety). Thus, in certain embodiments, such an IgG conserved glycosylation site is modified so as to reduce or eliminate glycosylation.

Other amino acid modifications of anti-CD3 antibodies or antigen-binding fragments thereof that result in reduced binding to at least one class of Fc (gamma) receptor are known in the art. For example, a humanized OTK3-derived antibody in which two amino acid residues at positions 234 and 235 of the Fc domain have been modified to alanine residues (referred to as hOKT3-gamma-1 (ala-ala)) is disclosed in United States Patent Publication numbers 2007/0077246 and 2008/0095766, the disclosures of which are incorporated herein by reference in their entirety. The hOKT3-gamma-1 (ala-ala) antibody is described as exhibiting reduced binding to Fc (gamma) receptors.

Other examples of anti-CD3 antibodies include, without limitation, hOKT3 (humanized (IgG1 or IgG4) anti-human CD3), HUM291 (humanized (IgG2) anti-human CD3; visilizumab; NUVION™), UCHT1 (mouse (IgG1) anti-human CD3), Leu4 (mouse (IgG1) anti-human CD3), 500A2 (hamster (IgG) anti-mouse CD3), CLB-T3/3 (mouse (IgG2a) anti-human CD3), BMA030 (mouse (IgG2a) anti-human CD3), YTH 12.5 (rat (IgG2b) anti-human CD3), and NI-0401 (fully human anti-human CD3). Those of ordinary skill in the art will be aware of other anti-CD3 antibodies and antigen binding fragments thereof that can be used in accordance with the dosing regimens disclosed herein.

In certain embodiments, an anti-CD3 antibody or antigen binding fragment thereof that exhibits reduced binding to at least one class of Fc (gamma) receptor is modified in that it lacks some or all of an Fc domain. For example, Fab fragments and F(ab′)₂ fragments lack some or all of an Fc domain.

In certain embodiments, an anti-CD3 antibody or antigen binding fragment thereof is modified in some other way such that it exhibits reduced binding to at least one class of Fc (gamma) receptor. For example, the anti-CD3 antibody or fragment may be modified by covalently linkage of a chemical moiety that prevents the anti-CD3 antibody or fragment from binding at least one class of Fc (gamma) receptor. As another example, the anti-CD3 antibody or fragment may be modified by non-covalently linkage of a chemical moiety that prevents the anti-CD3 antibody or fragment from binding at least one class of Fc (gamma) receptors. Any of a variety of moieties may be covalently or non-covalently linked to the anti-CD3 antibody or fragment to prevent binding to at least one class of Fc (gamma) receptor. Those of ordinary skill in the art will be aware of suitable moieties that can be linked to an antibody or fragment, and will be able to employ such moieties in accordance with the teachings herein.

Those of ordinary skill in the art will be aware of other anti-CD3 antibodies and antigen binding fragments thereof that exhibit reduced binding to at least one class of Fc (gamma) receptor, which antibodies and fragments can be employed in one or more of the dosing regimens disclosed herein.

### Pharmaceutical Formulations

Anti-CD3 antibodies or antigen binding fragments thereof described herein may be formulated for delivery by any available route including, but not limited to parenteral (e.g., intravenous, intradermal, or subcutaneous), oral, nasal, bronchial, opthalmic, transdermal (topical), transmucosal, rectal, and vaginal routes. The anti-CD3 antibody or fragment containing compositions may include a delivery agent (e.g., a cationic polymer, peptide molecular transporter, surfactant, etc., as described above) and/or a pharmaceutically acceptable carrier. As used herein the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into pharmaceutical formulations that comprise an antibody or fragment as described herein.

A pharmaceutical composition is formulated to be compatible with its intended route of administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Pharmaceutical formulations are ideally stable under the conditions of manufacture and storage and should be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be advantageous to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the anti-CD3 antibody or antigen binding fragment thereof in the required amount in an appropriate solvent with one or a combination of ingredients enumerated above, as required, followed by filtered sterilization. Generally, dispersions are prepared by incorporating the purified anti-CD3 antibody or fragment into a sterile vehicle which contains a basic dispersion medium and the required other ingredients from those enumerated above. In the case of sterile powders for the preparation of sterile injectable solutions, exemplary methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the anti-CD3 antibody or antigen binding fragment thereof can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the anti-CD3 antibody or antigen binding fragment thereof and a delivery agent are preferably delivered in the form of an aerosol spray from a pressured container or dispenser which contains a suitable propellant, e.g., a gas such as carbon dioxide, or a nebulizer. The present disclosure particularly contemplates delivery of the compositions using a nasal spray, inhaler, or other direct delivery to the upper and/or lower airway. Intranasal administration of DNA vaccines directed against influenza viruses has been shown to induce CD8+ T cell responses, indicating that at least some cells in the respiratory tract can take up DNA when delivered by this route, and the delivery agents of the invention will enhance cellular uptake. According to certain embodiments, the anti-CD3 antibody or fragment and a delivery agent are formulated as large porous particles for aerosol administration.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the purified polypeptide or protein and delivery agents are formulated into ointments, salves, gels, or creams as generally known in the art.

In certain embodiments, compositions are prepared with carriers that will protect the anti-CD3 antibody or antigen binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, the disclosure of which is incorporated herein by reference in its entirety.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active anti-CD3 antibody or antigen binding fragment thereof calculated to produce the desired therapeutic effect in association with the required pharmaceutical carrier.

The anti-CD3 antibody or antigen binding fragment thereof can be administered at various intervals and over different periods of time as required, e.g., one time per week for between about 1 to 10 weeks, between 2 to 8 weeks, between about 3 to 7 weeks, about 4, 5, or 6 weeks, etc. Those of ordinary skill in the art will appreciate that certain factors can influence the dosage and timing required to effectively treat a subject, including but not limited to the severity of the disease or disorder, previous treatments, the general health and/or age of the subject, and other diseases present. Generally, treatment of a subject with an anti-CD3 antibody or fragment as described herein can include a single treatment or, in many cases, can include a series of treatments. It is furthermore understood that appropriate doses may depend upon the potency of the anti-CD3 antibody or fragment and may optionally be tailored to the particular recipient, for example, through administration of increasing doses until a preselected desired response is achieved. It is understood that the specific dose level for any particular animal subject may depend upon a variety of factors including the activity of the specific polypeptide or protein employed, the age, body weight, general health, gender, and diet of the subject, the time of administration, the route of administration, the rate of excretion, any drug combination, and the degree of expression or activity to be modulated.

Pharmaceutical formulations as described herein can be included in a container, pack, or dispenser together with instructions for administration.

### Combination Therapies

Anti-CD3 antibodies and antigen binding fragments thereof can be administered according to one or more dosing regimens disclosed herein in combination with one or more other therapeutic agents. Therapeutic agents that can be administered in combination with an anti-CD3 antibody or fragment thereof include, but are not limited to, peptides, polypeptides, conjugates, nucleic acid molecules (e.g., DNA or RNA), small molecules, mimetic agents, synthetic drugs, inorganic molecules, and organic molecules.

In certain embodiments, a therapeutic agent to be used in combination with an anti-CD3 antibody or antigen binding fragment thereof is an immunomodulatory agent. Any of a variety of immunomodulatory agent known to those of skill in the art may be administered in combination with an anti-CD3 antibody or fragment, as disclosed herein. Immunomodulatory agents typically affect one or more aspects of an immune response in a subject including, without limitation, an inflammatory response, a complement cascade, leukocyte and lymphocyte differentiation, proliferation, and/or effector function, monocyte and/or basophil counts, and the cellular communication among cells of the immune system. Non-limiting examples of immunomodulatory agents include proteinaceous agents such as cytokines, peptide mimetics, and antibodies (e.g., human, humanized, chimeric, monoclonal, polyclonal, Fvs, scFvs, Fab or F(ab′)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide). Other examples of immunomodulatory agents can be found, e.g., in United States Patent Publication Number 2005/0002934 A1 at paragraphs 259-275 which is incorporated herein by reference in its entirety. In certain embodiments, an immunomodulatory agent is a chemotherapeutic agent. In certain embodiments, an immunomodulatory agent is an immunomodulatory agent other than a chemotherapeutic agent.

In certain embodiments, a therapeutic agent administered in combination with an anti-CD3 antibody or antigen binding fragment thereof is useful in the prevention or treatment of an immune-related disease. For example, such a therapeutic agent may be useful in preventing, treating, delaying the onset of, slowing the progression of or ameliorating one or more symptoms associated with an immune-related disease. In certain embodiments, a therapeutic agent administered in combination with an anti-CD3 antibody or fragment prevents or treats the same immune-related disease as is prevented or treated by the anti-CD3 antibody or fragment. In certain embodiments, a therapeutic agent administered in combination with an anti-CD3 antibody or fragment prevents or treats a different immune-related disease as is prevented or treated by the anti-CD3 antibody or fragment.

Any therapeutic agent that prevents or treats one or more symptoms associated with an immune-related disease can be used in combination with an anti-CD3 antibody or antigen binding fragment thereof. Examples of such therapeutic agents include, but are not limited to antibody fragments, GLP-1 analogs or derivatives, GLP-1 agonists (e.g. exendin-4; exentatide), amylin analogs or derivatives, insulin, dermatological agents for rashes and swellings (e.g., phototherapy (i.e., ultraviolet B radiation), photochemotherapy (e.g., PUVA) and topical agents such as emollients, salicylic acid, coal tar, topical steroids, topical corticosteroids, topical vitamin D3 analogs (e.g., calcipotriene), tazarotene, and topical retinoids), anti-inflammatory agents (e.g., corticosteroids (e.g., prednisone and hydrocortisone), glucocorticoids, steroids, non-steroidal anti-inflammatory drugs (e.g., aspirin, ibuprofen, diclofenac, and COX-2 inhibitors), beta-agonists, anticholinergic agents and methyl xanthines), immunomodulatory agents (e.g., small organic molecules, a T cell receptor modulators, cytokine receptor modulators, T cell depleting agents, cytokine antagonists, monokine antagonists, lymphocyte inhibitors, or anti-cancer agents), gold injections, sulphasalazine, penicillamine, anti-angiogenic agents (e.g., angiostatin, TNF-alpha antagonists (e.g., anti-TNF-alpha antibodies), and endostatin), dapsone, psoralens (e.g., methoxalen and trioxsalen), anti-malarial agents (e.g., hydroxychloroquine), anti-viral agents, and antibiotics (e.g., erythomycin and penicillin).

In certain embodiments, a therapeutic agent to be used in combination with an anti-CD3 antibody or antigen binding fragment thereof is administered to a patient according to the same dosing regimen as the anti-CD3 antibody or fragment. For example, if a particular dosing regimen calls for an anti-CD3 antibody or fragment to be administered to a patient on five consecutive days, a therapeutic agent may also be administered to the patient on the same five consecutive days. The particular dose of the therapeutic agent to be administered can be chosen by those of ordinary skill in the art based on any of a variety of factors, including for example, that therapeutic agent's known effective dose, pharmacokinetic and/or pharmacodynamic interactions between the anti-CD3 antibody or fragment and the therapeutic agent, and the like.

In certain embodiments, a therapeutic agent to be used in combination with an anti-CD3 antibody or antigen binding fragment thereof is administered to a patient according to a different dosing regimen as the anti-CD3 antibody or fragment. For example, if a particular dosing regimen calls for an anti-CD3 antibody or fragment to be administered to a patient on five consecutive days, a therapeutic agent may also be administered to the patient on only one day, or on two, three, four, six, seven, eight or more consecutive days, or on non-consecutive days. Those of ordinary skill in the art will be aware of suitable dosing regimens for a given therapeutic agent and will be able to administer such a therapeutic agent to a patient according to that therapeutic agent's effective dosing regimen.

In certain embodiments, analgesics, anti-histamines, anti-inflammatories and/or antiemetics can be administered before, after, and/or during a treatment regimen in order to improve tolerability. Those skilled in the art will recognize other such compounds. These agents can be used in combination with any of the dosage regimens, including dose escalation, described herein.

In some embodiments, other therapies and compounds for treatment of immune related diseases can be continued during and/or after the treatment regimen as needed. For example, insulin therapy can be continued for a diabetic patient to control glycemic excursions. Similarly, a patient with rheumatoid arthritis can continue an ongoing therapy such as methotrexate, prednisolone and/or other medications which may also be used to treat patients for the purpose of pain relief only, to reduce joint inflammation and/or to help slow or prevent joint damage.

Certain embodiments of methods and compositions provided herein are further illustrated by the following examples. The examples are provided for illustrative purposes only. They are not to be construed as limiting the scope or content of the invention in any way.

### EXAMPLES

### Example 1

### Methods

Determination of TRX4 Serum Levels:

Serum levels of otelixizumab were determined by an ELISA assay conducted under good laboratory practices (GLP). Blood samples were collected before infusion of otelixizumab, at the end of each infusion, and 2 hours after the end of infusion. The ELISA assay used two anti-otelixizumab monoclonal antibodies, one as the capture antibody and the second as the bridging antibody. The limit of quantitation (LOQ) of this assay is 0.0199 μg/mL.

Assessment of Circulating Lymphocyte Phenotype and Number:

Flow cytometry immunophenotyping was used to monitor changes in peripheral blood lymphocytes and subsets of total T cells, CD4+ T cells and CD8+ T cells. CD19+ B cells also were monitored as B cells are targets of Epstein Barr virus (EBV), and EBV reactivation was seen in the Phase II study conducted in the EU. (Keymeulen, 2005). These analyses would therefore detect any anbnormal EBV-induced B cell proliferation. No significant EBV reactivation was observed.

Method of Calculation of Absolute Lymphocyte Counts:

Absolute counts for each lymphocyte subset per liter were calculated based on CD markers by multiplying the absolute number of lymphocytes per liter by the percentage of lymphocytes in the lymphocyte flow cytometry gate (as determined using forward and side light scatter parameters) bearing the CD marker of interest. To facilitate accurate enumeration of lymphocyte populations that occur at low frequencies, 50,000 events were collected by flow cytometry. The absolute number of lymphocytes was determined by multiplying the total white blood cell (WBC) count (from a hematology sample taken at the same time as the flow cytometry sample) by the percentage of lymphocytes as determined by the WBC differential cell count. Absolute counts and percentages were calculated for each parameter, and changes from baseline were determined for each post-baseline assessment.

Detection of Otelixizumab Bound to CD4+ and CD8+ T Cells:

Cell-bound otelixizumab was detected on CD4+ and CD8+ T cells using a fluorochrome-conjugated anti-human IgG antibody reagent. Fluorescence intensity was quantified by using standard units known as Molecules of Equivalent Soluble Fluorochrome (MESF). MESF units were determined by comparing the fluorescence intensity signal from a microbead standard to the signal from the sample solution stained with the same fluorochrome. There is a direct relationship between the MESF value of a cell population and the number of binding antibodies. Use of MESF standardizes data collected on different days. The MESF of the anti-human IgG was used to quantify the amount of cell-bound otelixizumab.

CD3/T Cell Receptor (TCR) Complex Analysis—CD3/TCR Modulation and Saturation:

CD3 proteins are components of the CD3/TCR complex. CD4+ T cells express approximately twice the number of CD3/TCR complexes as CD8+ T cells. To evaluate the level of surface expression of the CD3/TCR complex and its modulation, CD3/TCR expression was determined for both CD4+ and CD8+ T cells using the noncompeting anti-TCR antibody BMA031. (Abcam, Inc., Cambridge, Mass.; Borst, et al., Hum. Immunol., Vol. 29, pgs. 175-188 (1990)). Binding of this antibody is not blocked by otelixizumab bound to the CD3 surface molecule when otelixizumab serum levels are below 1 μg/mL. Serum levels of otelixizumab greater than 1 μg/mL were not detected in any of subjects described in this example. The MESF of the anti-TCR antibody was used to quantify the number of CD3/TCR complexes present on T cells. Free otelixizumab binding sites (unoccupied by previously administered otelixizumab) were detected by staining with biotinylated otelixizumab and fluorophore-conjugated streptavidin. The MESF of bound biotinylated otelixizumab is directly proportional to the availability of free otelixizumab binding sites.

Determination of Peak Cytokine Levels:

Levels of the cytokines interleukin 6 (IL6) an tumor necrosis factor-alpha (TNF-alpha) were determined by an ELISA assay. Blood samples were collected one hour after the end of infusion on each of day of the dosing regimen. The ELISA assay used anti-IL6 and anti-TNF-alpha antibodies. The highest daily cytokine levels for each of IL6 and TNF-alpha were recorded.

Determination of Perturbation of EBV Immunity:

Perturbation of EBV immunity was determined by quantitative PCR to detect EBV viral copy number.

Determination of Immunogenicity:

Patient immune response to the administered otelixizumab was measured by a bridging ELISA assay. Blood samples were collected one hour after the end of infusion on each of day of the dosing regimen. Antibodies to otelixizumab were measured at Baseline and at specified post-baseline visits (Day 28 and month 3) using enzyme-linked immunosorbent assays (ELISAs). Samples were analyzed using SOP-PC-006-01, which is a GLP bridging ELISA, capable of identifying antibodies that are made to the entire otelixizumab molecule. This was done by coating well bottoms of ELISA microtiter plates with otelixizumab, adding test serum samples (or a standard antibody sample) to the wells and incubating the plates, followed by the addition of biotinylated otelixizumab to the wells and incubating the plates, followed by detection of the bound biotinylated otelixizumab with horseradish peroxidase-conjugated streptavidin. Each incubation step was followed by thorough washing of the wells to remove unbound material. Titer results for test samples are determined as concentrations in μg/mL based on extrapolation of the data to a standard curve. The LOQ (limit of quantitation) of the assay is 0.75 μg/mL. The percentage of patients that exhibited increased levels of endogenous antibodies to otelixizumab was determined.

### Example 2

### Dosing Regimen CH2

Otelixizumab (TRX4) is an anti-CD3 antibody having a humanized heavy chain (containing rat heavy chain variable (VH) CDRs 1, 2 and 3, four human VH framework regions, and a human IgG1 constant region), a chimeric light chain (containing a rat light chain variable region (VL) and a human light chain kappa constant region), and has an aglycosylated Fc region, in which Asn297 of SEQ ID NO: 1 has been mutated to A1a297. Residue numbers are given according to the Kabat canonical numbering system (see Kabat E A, Wu T T, Perry H, Gottesman K, and Foeller C. (1991) Sequences of Proteins of Immunological Interest, Fifth Edition. NIH Publication No. 91-3242, incorporated herein by reference in its entirety). Otelixizumab was administered intravenously to a cohort of 35 patients diagnosed with Type I diabetes according to the following dosing schedule: 0.1 mg on day 1, 0.2 mg on day 2, 0.3 mg on day 3, and 0.75 mg on days 4-8. Daily doses were administered approximately 24 hours apart, and each dose was administered by intravenous infusion over a course of between about fifteen minutes and about two hours. Pharmacokinetic (PK) and pharmacodynamic (PD) parameters of otelixizumab were evaluated immediately prior to (descriptions on the X axis labeled “Pre”) and immediately after (descriptions on the X axis labeled “EOI”) each daily dose.

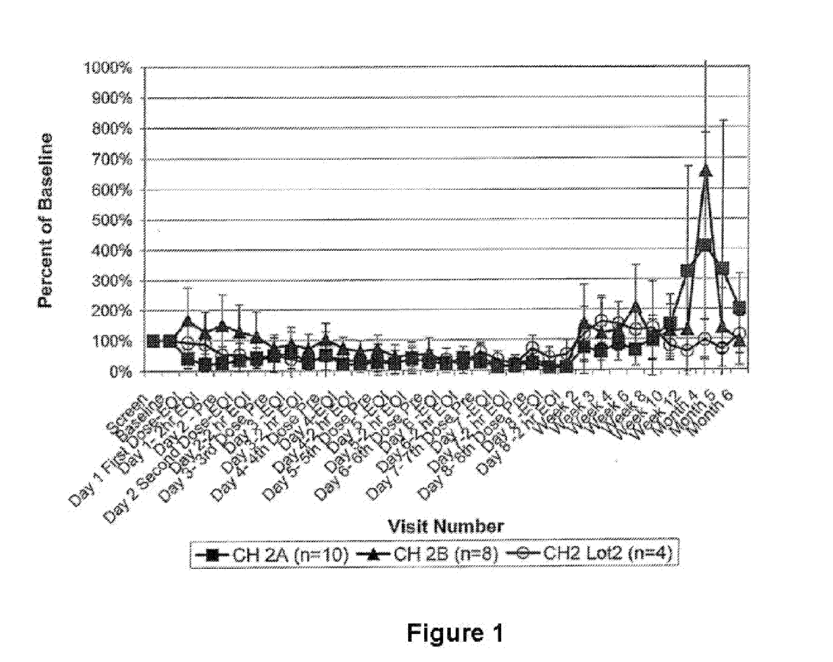

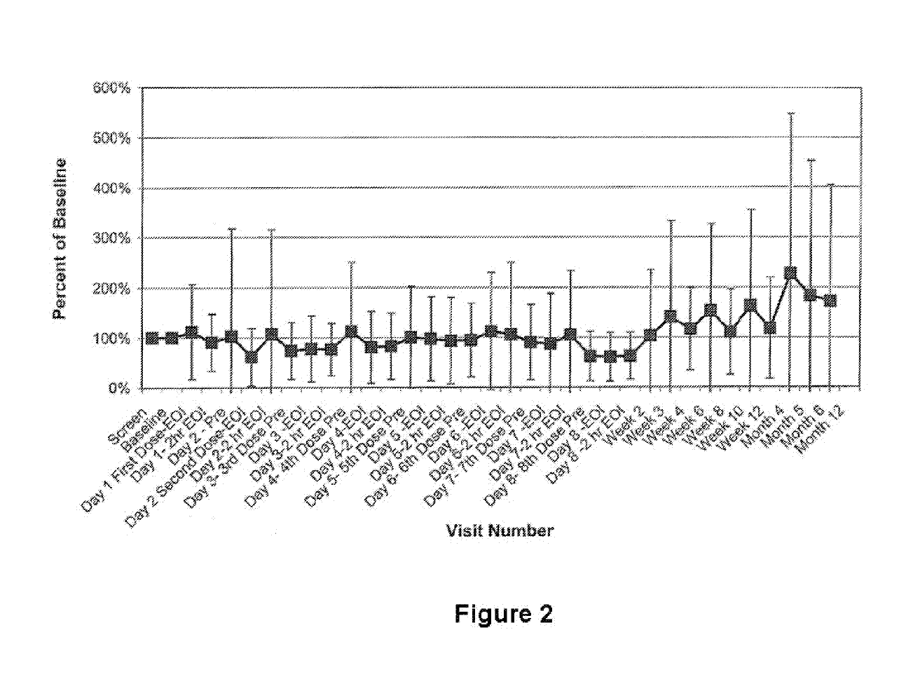

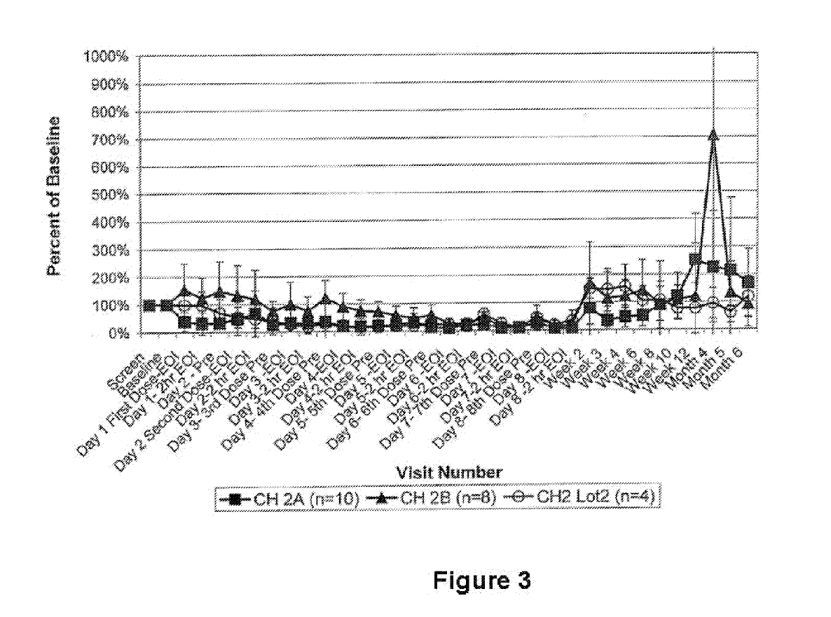

Absolute counts for CD4+FoxP3+ T cells, CD8+FoxP3+ T cells, and CD4+CD25+FoxP3+ T cells were determined based on CD markers as described above. Absolute counts and percentages were calculated for each parameter, and changes from baseline were determined for each post-baseline assessment. CD4+FoxP3+ T cell results for dosing regimen CH2 are shown in FIG. 1. CD8+FoxP3+ T cell results for dosing regimen CH2 are shown in FIG. 2. CD4+CD25+FoxP3+ T cell results for dosing regimen CH2 are shown in FIG. 3. As shown in FIGS. 1, 2 and 3, a transient decrease of both CD4+ and CD8+ T-lymphocytes in peripheral blood (lymphopenia) was observed during dosing. The number of lymphocytes recovered to baseline levels between week 2 and week 3. FIGS. 1-3 show that CD4+FoxP3+ and CD4+CD25+FoxP3+ T cells increased and/or proliferated, while the CD8+FoxP3+ T cells did not.

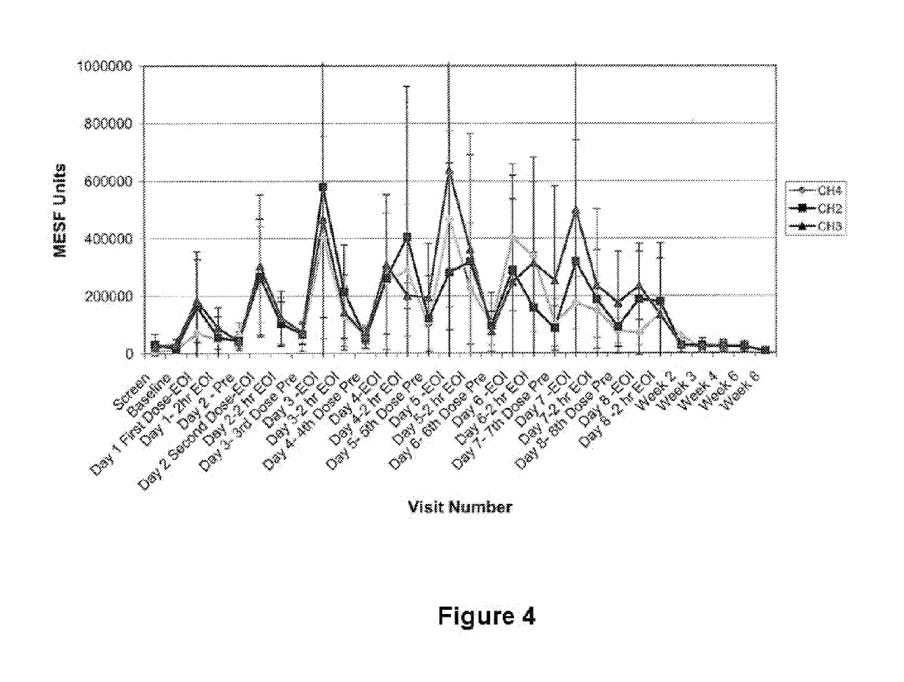

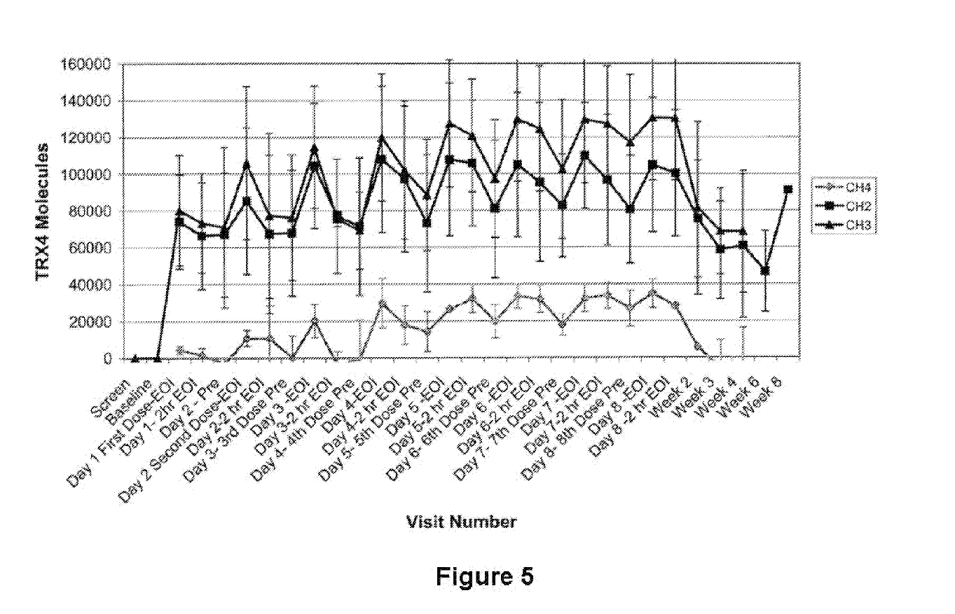

Cell-bound otelixizumab on CD4+ T cells and CD8+ T-cells was determined using anti-human IgG antibody reagents, and fluorescence intensity was quantified by using standard MESF units as described above. Results for dosing regimen CH2 are shown in FIGS. 4 and 5, in the line indicated by square data points, labeled as CH2. FIG. 4 shows MESF units, while FIG. 5 shows number of cell bound otelixizumab molecules. When the infusions of otelixizumab were stopped after Day 8, otelixizumab binding levels returned to baseline between week 2 and week 3.

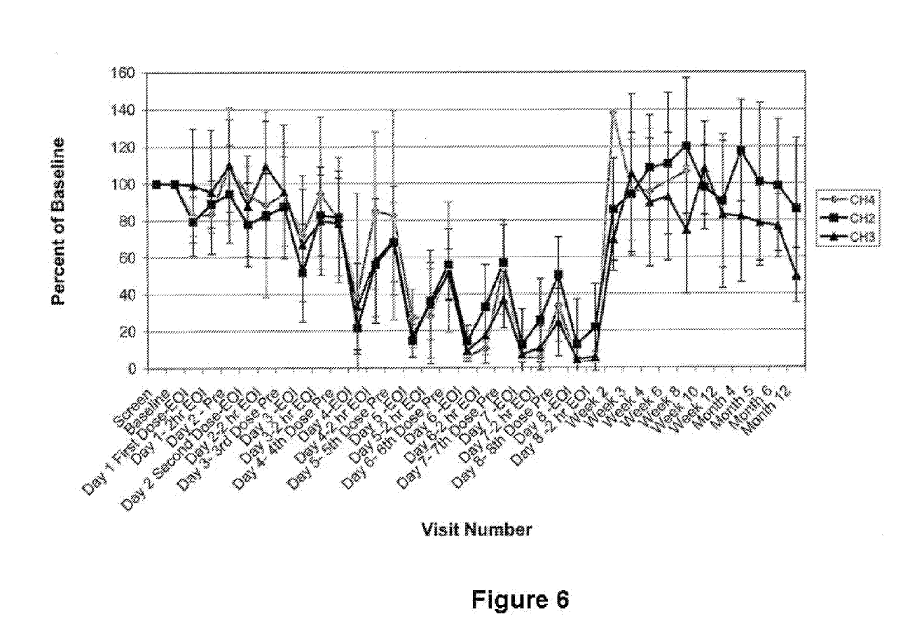

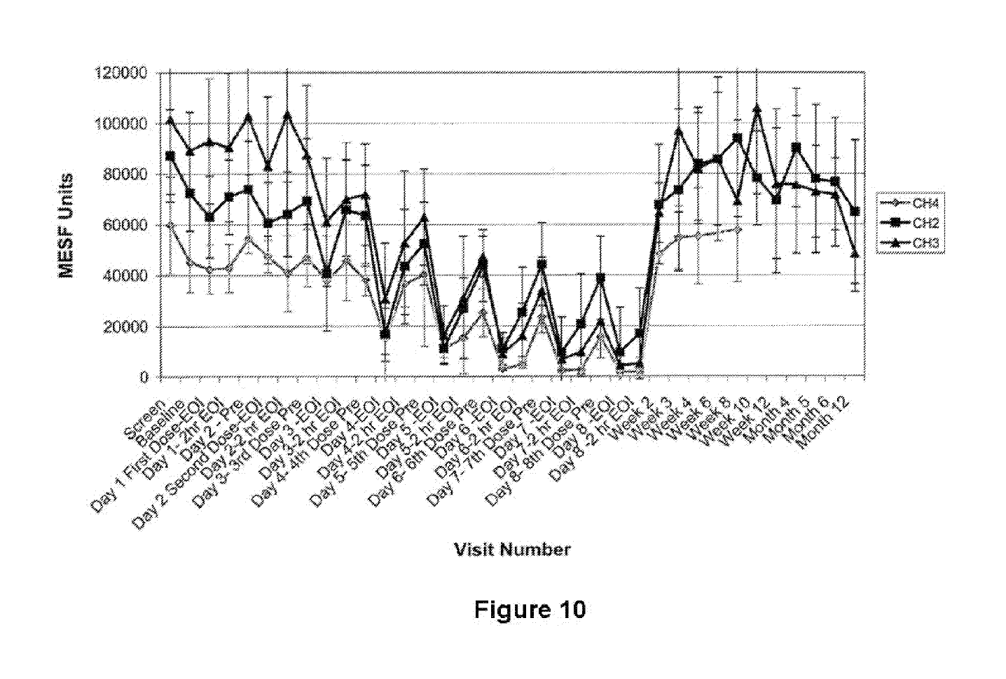

The number of CD3/TCR sites on CD4+ T cells was determined using the noncompeting anti-TCR antibody BMA031 as described above. Results for dosing regimen CH2 are shown in FIGS. 6 and 10, in the lines indicated by square data points, labeled as CH2. FIG. 6 shows percent CD3/TCR sites compared to baseline. FIG. 10 shows the number of CD3/TCR sites expressed as MESF units. As can be seen, a transient decrease in the number of CD4+ T cell CD3/TCR sites was observed during dosing. When the infusions of otelixizumab were stopped after Day 8, the number of CD3/TCR sites returned to baseline between week 2 and week 3.

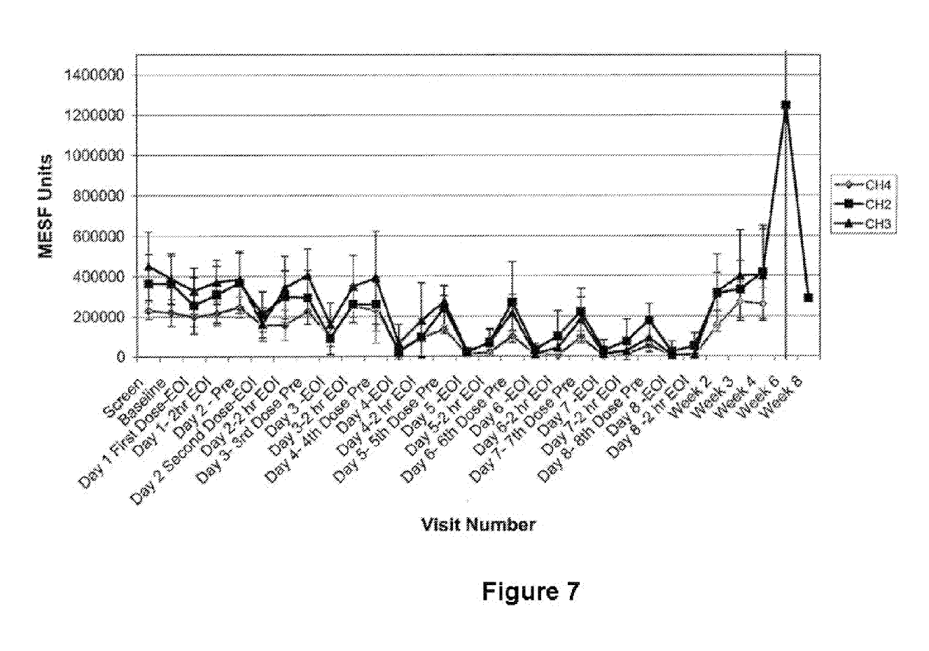

The number of free CD3 sites on CD4+ T cells was determined using biotinylated otelixizumab as described above. Results for dosing regimen CH2 are shown in FIG. 7, in the line indicated by square data points, labeled as CH2. As can be seen, a transient decrease in the number of free CD3 sites was observed during dosing. When the infusions of otelixizumab were stopped after Day 8, the number of free CD3 sites returned to baseline between week 2 and week 3.

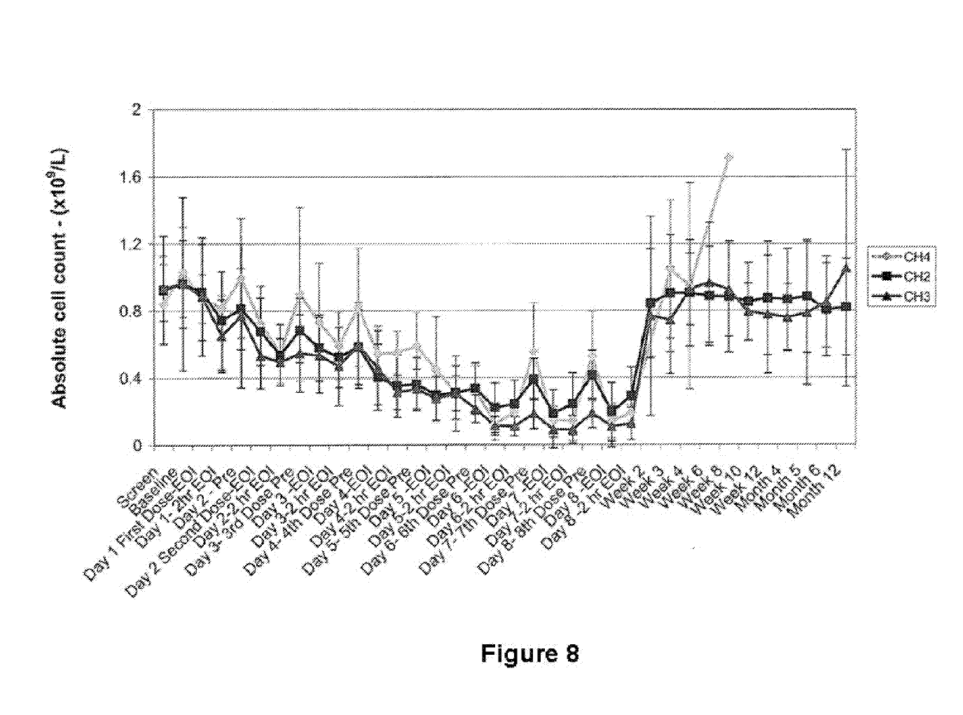

Absolute counts for CD4+ T cells were determined based on CD markers as described above. Results for dosing regimen CH2 are shown in FIG. 8, in the line indicated by square data points, labeled as CH2. As shown in FIG. 8, a transient decrease of CD4+ T cells in peripheral blood was observed during dosing. The number of CD4+ T cells recovered to baseline levels between week 2 and week 3.

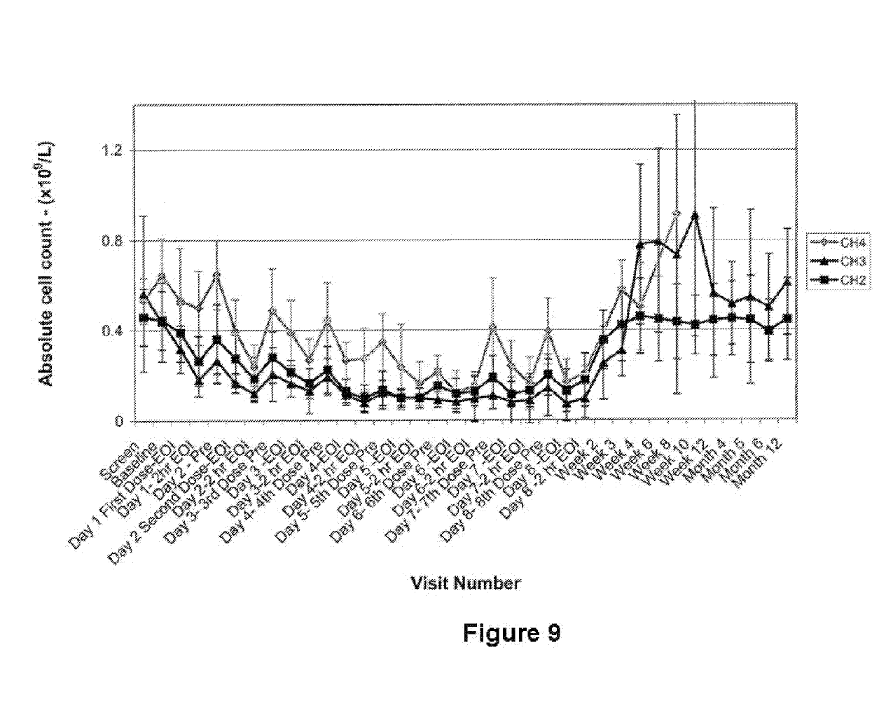

Absolute counts for CD8+ T cells were determined based on CD markers as described above. Results for dosing regimen CH2 are shown in FIG. 9, in the line indicated by square data points, labeled as CH2. As shown in FIG. 9, a transient decrease of CD8+ T cells in peripheral blood was observed during dosing. The number of CD8+ T cells recovered to baseline levels between week 2 and week 3.

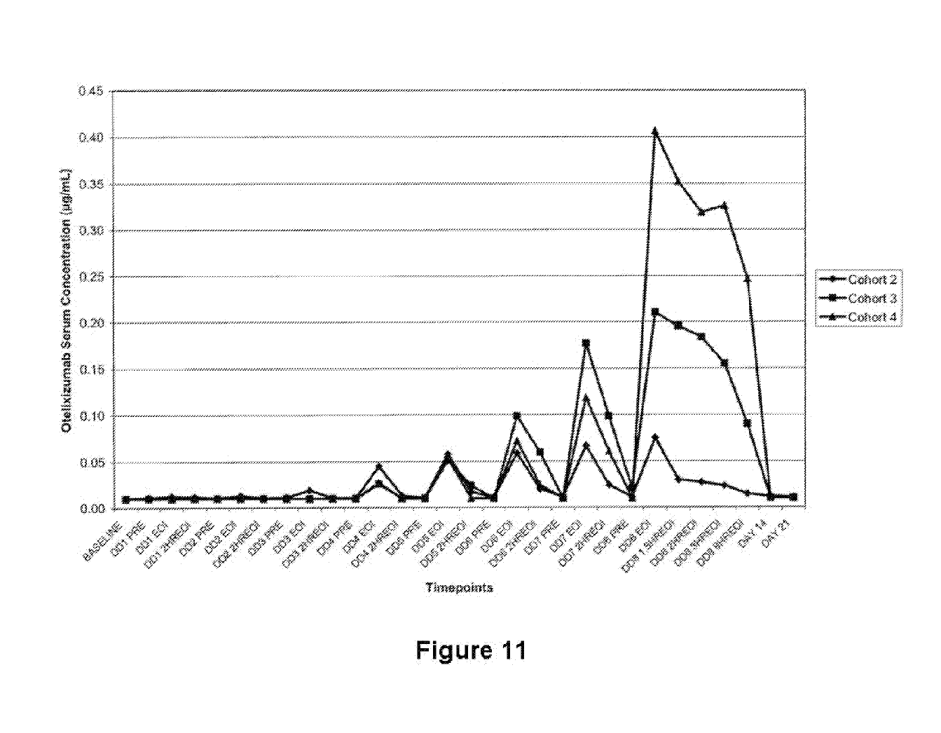

Serum levels of otelixizumab were determined by ELISA assay as described above. Results for dosing regimen CH2 are shown in FIG. 11, in the line indicated by diamond data points, labeled as CH2. As shown in FIG. 11, serum levels of otelixizumab rose after each daily administration, and then dropped back to baseline by the next day. Peak levels of the cytokines IL6 and TNF-alpha, perturbation of EBV immunity, and immunogenicity were determined as described above. The peak level for any one regimen is the highest level seen in any subject on any day of the relevant regimen. Results are shown in Table 2.

TABLE 2
Pharmacodynamic Profile of Various Otelixizumab Dosing Regimens

| | | Total | | | | | |
|---|---|---|---|---|---|---|---|
| | | Dose | | Peak | Peak TNF- | Perturbation of EBV | Immuno- |
| | Daily Doses (mg) | (mg) | n | IL6 (pg) | alpha (pg) | Immunity (PEI) | genicity (%) |
| BDR* | 8 × 6 | 48 | 37 | ~3100 | ~1250 | Yes | 75 |
| Cohort 1 | 0.1 × 3 | 0.3 | 4 | 20.24 | 51.63 | No | 0 |
| Cohort 2 | 0.5 × 3 | 1.5 | 3 | 329.92 | 182.97 | No | 33.3 |
| Cohort 9 | 0.1, 0.3, 0.5 | 0.9 | 4 | 55.81 | 71.02 | No | 0 |
| Cohort 10 | 0.3, 0.5, 1.0 | 1.8 | 1 | 79.76 | 30.46 | No | 0 |
| Cohort A | 0.1, 0.2, 0.3, 0.5 | 1.1 | 6 | 32.48 | 15.94 | No | 0 |
| Cohort A (1/2) | 0.05, 0.1, 0.15, 0.25 | 0.55 | 1 | 45.04 | 6.09 | No | 0 |
| Cohort B | 0.1, 0.2, 0.3, 0.75 | 1.35 | 4 | 48.52 | 43.82 | No | 0 |
| Cohort C | 0.1, 0.2, 0.3, 1.0 | 1.6 | 4 | 36.79 | 27.11 | No | 50 |
| CH1 | 0.1, 0.2, 0.3, 0.5 × 5 | 3.1 | 15 | 269.41 | 103.51 | No | 0 |
| CH2 | 0.1, 0.2, 0.3, 0.75 × 5 | 4.35 | 35 | 99.1 | 43 | No | 0 |
| CH3 | 0.1, 0.2, 0.3, 0.75, 1, | 6.85 | 6 | 358.9 | 50.9 | No | 50 |
| | 1.25, 1.5, 1.75 | | | | | | |
| CH4 | 0.1, 0.2, 0.3, 0.75, 1, | 8.85 | 5 | 316.8 | 157.24 | No | 80 |
| | 1.25, 1.5, 3.75 | | | | | | |
| CH5 | 0.2, 0.4, 0.6, 0.8, 1.1 | 3.1 | N/A | N/A | N/A | N/A | N/A |

*Described in Keymeulen et al., N Engl J Med. 23; 352(25): 2598-608, 2005
“N/A”, not available

Two subjects that were administered the CH2 dosing regimen within 93 days of being diagnosed with new-onset type I diabetes mellitus. Each of these subjects exhibited c-peptide levels greater than 0.2 nmol/L. At twelve months following treatment, these two subjects exhibited a mean change in c-peptide levels of −24.7%, with a standard deviation of 20.23. The subjects' c-peptide levels were improved compared to the 40-100% decrease observed by others at twelve months in subjects that did not undergo treatment (see e.g., Palmer J. P., Diabetes Metab Res Rev 2009, incorporated herein by reference in its entirety).

This Example shows that the CH2 dosing regimen results in partial modulation of CD3/TCR sites during dosing and that regulatory T cells are induced after dosing. This example also shows that lymphopenia is sustained throughout the course of the dosing regimen, but rebounds to baseline levels in the weeks following the end of the regimen.

### Example 3

### Dosing Regimen CH3

Otelixizumab was administered intravenously to a cohort of 6 patients diagnosed with Type I diabetes according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Daily doses were administered approximately 24 hours apart, and each dose was administered by intravenous infusion over a course of about two hours. Pharmacokinetic (PK) and pharmacodynamic (PD) parameters of otelixizumab were evaluated immediately prior to (descriptions on the X axis labeled “Pre”) and immediately after (descriptions on the X axis labeled “EOI”) each daily dose.

Cell-bound otelixizumab on CD4+ T cells and CD8+ T-cells was determined using anti-human IgG antibody reagents, and fluorescence intensity was quantified by using standard MESF units as described above. Results for dosing regimen CH3 are shown in FIGS. 4 and 5, in the line indicated by triangle data points, labeled as CH3. FIG. 4 shows MESF units, while FIG. 5 shows number of cell bound otelixizumab molecules. When the infusions of otelixizumab were stopped after Day 8, otelixizumab binding levels returned to baseline between week 2 and week 3.

The number of CD3/TCR sites on CD4+ T cells was determined using the noncompeting anti-TCR antibody BMA031 as described above. Results for dosing regimen one are shown in FIGS. 6 and 10, in the lines indicated by triangle data points, labeled as CH3. FIG. 6 shows percent CD3/TCR sites compared to baseline. FIG. 10 shows the number of CD3/TCR sites expressed as MESF units. As can be seen, a transient decrease in the number of CD4+ CD3/TCR sites was observed during dosing. When the infusions of otelixizumab were stopped after Day 8, the number of CD3/TCR sites returned to baseline between week 2 and week 3.

The number of free CD3 sites on CD4+ T cells was determined using biotinylated otelixizumab as described above. Results for dosing regimen CH3 are shown in FIG. 7, in the line indicated by triangle data points, labeled as CH3. As can be seen, a transient decrease in the number of free CD3 sites was observed during dosing. When the infusions of otelixizumab were stopped after Day 8, the number of free CD3 sites returned to baseline between week 2 and week 3.

Absolute counts for CD4+ T cells were determined based on CD markers as described above. Results for dosing regimen CH3 are shown in FIG. 8, in the line indicated by triangle data points, labeled as CH3. As shown in FIG. 8, a transient decrease of CD4+ T cells in peripheral blood was observed during dosing. The number of CD4+ T cells recovered to baseline levels between week 2 and week 3.

Absolute counts for CD8+ T cells were determined based on CD markers as described above. Results for dosing regimen CH3 are shown in FIG. 9, in the line indicated by triangle data points, labeled as CH3. As shown in FIG. 9, a transient decrease of CD8+ T cells in peripheral blood was observed during dosing. The number of CD8+ T cells recovered to baseline levels between week 2 and week 3.

Serum levels of otelixizumab were determined by ELISA assay as described above. Results for dosing regimen CH3 are shown in FIG. 11, in the line indicated by square data points, labeled as CH3. As shown in FIG. 11, serum levels of otelixizumab rose after each daily administration, and then dropped back to baseline by the next day.

Peak cytokine profiles of IL6 and TNF-alpha, perturbation of EBV immunity, and immunogenicity were determined as described above. Results are shown in Table 2. As can be seen from Table 2, peak cytokine profiles of IL6 were similar to those observed in the Cohort 2 dosing regimen while peak cytokine levels of TNF-alpha were reduced, despite the significantly higher daily doses administered on the later days of the CH3 regimen. No perturbation of EBV immunity was observed. 50% of the patients exhibited immunogenicity to otelixizumab.

Two subjects that were administered the CH3 dosing regimen within 93 days of being diagnosed with new-onset type I diabetes mellitus. Each of these subjects exhibited c-peptide levels greater than 0.2 nmol/L. At twelve months following treatment, these two subjects exhibited a mean change in c-peptide levels of −19.0%, with a standard deviation of 17.06. The subjects' c-peptide levels were improved compared to the 40-100% decrease observed by others at twelve months in subjects that did not undergo treatment (see e.g., Palmer J. P., Diabetes Metab Res Rev 2009, incorporated herein by reference in its entirety).

This Example shows that the CH3 dosing regimen results in partial modulation of CD3/TCR sites during dosing and that regulatory T cells are induced after dosing. These effects were more pronounced in subjects administered the CH3 dosing regimen that in subjects administered the CH2 dosing regimen. This example also shows that lymphopenia is sustained throughout the course of the dosing regimen, but rebounds to baseline levels in the weeks following the end of the regimen.

### Example 4

### Dosing Regimen CH4

Otelixizumab was administered intravenously to a cohort of 5 patients diagnosed with Type I diabetes according to the following dosing schedule: 0.1 mg on day 1, 0.2 mg on day 2, 0.3 mg on day 3, 0.75 mg on day 4, 1.0 mg on day 5, 1.25 mg on day 6, 1.5 mg on day 7, and 3.75 mg on day 8. Daily doses were administered approximately 24 hours apart, and each dose was administered by intravenous infusion over a course of about two hours, except for the final 3.75 mg dose, which was administered over a course of about four hours. Pharmacokinetic (PK) and pharmacodynamic (PD) parameters of otelixizumab were evaluated immediately prior to (descriptions on the X axis labeled “Pre”) and immediately after (descriptions on the X axis labeled “EOI”) each daily dose.

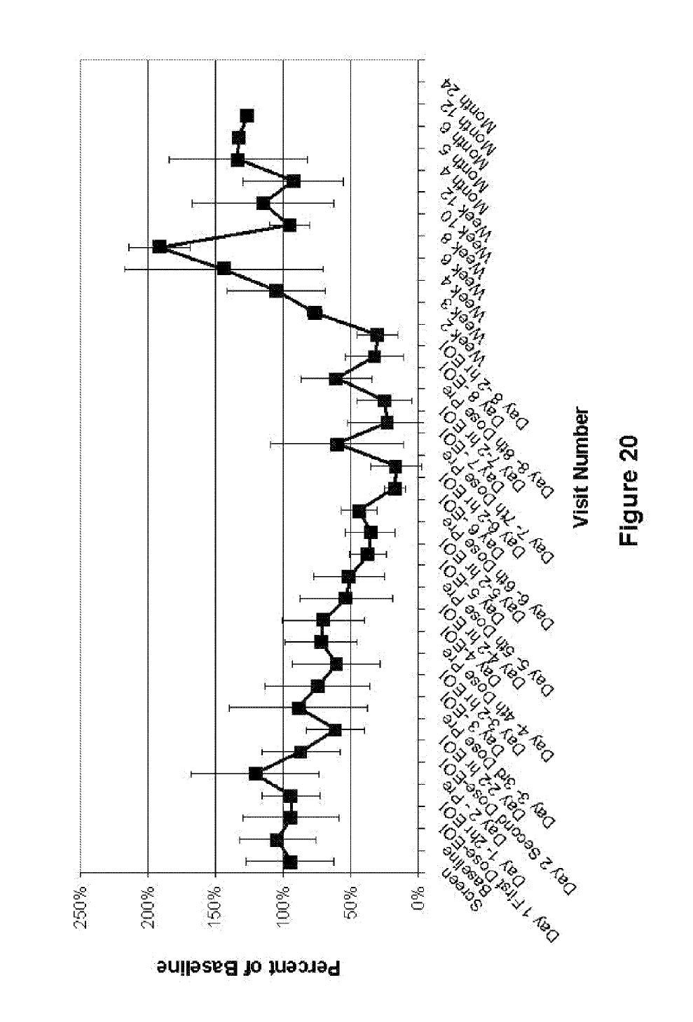

Absolute counts for CD4+CD25+FoxP3+ T cells were determined based on CD markers as described above. Absolute counts and percentages were calculated for each parameter, and changes from baseline were determined for each post-baseline assessment. CD4+CD25+FoxP3+ T cell results for dosing regimen CH4 are shown in FIG. 20. As shown in FIG. 3, a transient decrease of in these cells was observed during dosing. The number of lymphocytes recovered to baseline levels between week 6 and week 8. (FIG. 20) shows that CD4+CD25+FoxP3+ T cells increased.

Cell-bound otelixizumab on CD4+ T cells and CD8+ T-cells was determined using anti-human IgG antibody reagents, and fluorescence intensity was quantified by using standard MESF units as described above. Results for dosing regimen CH4 are shown in FIGS. 4 and 5, in the line indicated by diamond data points, labeled as CH4. FIG. 4 shows MESF units, while FIG. 5 shows number of cell bound otelixizumab molecules. When the infusions of otelixizumab were stopped after Day 8, otelixizumab binding levels returned to baseline between week 2 and week 3.

The number of CD3/TCR sites on CD4+ T cells was determined using the noncompeting anti-TCR antibody BMA031 as described above. Results for dosing regimen CH4 are shown in FIGS. 6 and 10, in the lines indicated by diamond data points, labeled as CH4. FIG. 6 shows percent CD3/TCR sites compared to baseline. FIG. 10 shows the number of CD3/TCR sites expressed as MESF units. As can be seen, a transient decrease in the number of CD4+ T cell CD3/TCR sites was observed during dosing. When the infusions of otelixizumab were stopped after Day 8, the number of CD3/TCR sites returned to baseline between week 2 and week 3.

The number of free CD3 sites on CD4+ T cells was determined using the noncompeting anti-TCR antibody BMA031 as described above. Results for dosing regimen CH4 are shown in FIG. 7, in the line indicated by diamond data points, labeled as CH4. As can be seen, a transient decrease in the number of free CD3 sites was observed during dosing. When the infusions of otelixizumab were stopped after Day 8, the number of free CD3 sites returned to baseline between week 2 and week 3.

Absolute counts for CD4+ T cells were determined based on CD markers as described above. Results for dosing regimen CH4 one are shown in FIG. 8, in the line indicated by diamond data points, labeled as CH4. As shown in FIG. 8, a transient decrease of CD4+ T cells in peripheral blood was observed during dosing. The number of CD4+ T cells recovered to baseline levels between week 2 and week 3.

Absolute counts for CD8+ T cells were determined based on CD markers as described above. Results for dosing regimen one are shown in FIG. 9, in the line indicated by diamond data points, labeled as CH4. As shown in FIG. 9, a transient decrease of CD8+ T cells in peripheral blood was observed during dosing. The number of CD8+ T cells recovered to baseline levels between week 2 and week 3. Serum levels of otelixizumab were determined by ELISA assay as described above. Results for dosing regimen CH4 are shown in FIG. 11, in the line indicated by triangle data points, labeled as CH4. As shown in FIG. 11, serum levels of otelixizumab rose after each daily administration, and then dropped back to baseline by the next day

Peak cytokine profiles of IL6 and TNF-alpha, perturbation of EBV immunity, and immunogenicity were determined as described above. Results are shown in Table 2. As can be seen from Table 2, peak cytokine profiles of IL6 and TNF-alpha were comparable to the Cohort 2 dosing regimen, despite the significantly higher daily doses administered on the later days of the CH4 regimen. No perturbation of EBV immunity was observed. 75% of the patients exhibited immunogenicity to otelixizumab. The relative levels of anti-otelixizumab antibody prior to the start of the CH4 study (Baseline), on day 28, and week 12 of the study for five patients are shown in Table 3.

TABLE 3
Levels of anti-otelixizumab antibody
in the five subjects from the CH4 study

| Subject | Baseline | Day 28 | Week 12 |
|---|---|---|---|
| 510003 | =0.08 μg/mL | 9.64 μg/mL | 18.4 μg/mL |
| 380018 | =0.08 μg/mL | =0.08 μg/mL | 0.15 μg/mL |
| 530002 | =0.08 μg/mL | 0.09 μg/mL | 3.53 μg/mL |
| 380026 | =0.08 μg/mL | 2.35 μg/mL | 37.8 μg/mL |
| 380020 | =0.08 μg/mL | 0.123 μg/mL | 0.214 μg/mL |

LOQ = 0.08 μg/mL

This Example shows that the CH4 dosing regimen results in partial modulation of CD3/TCR sites during dosing and that regulatory T cells are induced after dosing. These effects were more pronounced in subjects administered the CH4 dosing regimen that in subjects administered either the CH2 or the CH3 dosing regimen. This example also shows that lymphopenia is sustained throughout the course of the dosing regimen, but rebounds to baseline levels in the weeks following the end of the regimen.

### Example 5

### Dosing Regimen CH5

Otelixizumab was administered intravenously to a cohort of patients diagnosed with Type I diabetes according to the following dosing schedule: 0.2 mg on day 1, 0.4 mg on day 2, 0.6 mg on day 3, 0.8 mg on day 4, and 1.1 mg on day 5. Daily doses were administered approximately 24 hours apart, and each dose is administered by intravenous infusion over a course of about two hours. Pharmacokinetic (PK) and pharmacodynamic (PD) parameter of otelixizumab were evaluated immediately prior to and immediately after each daily dose.

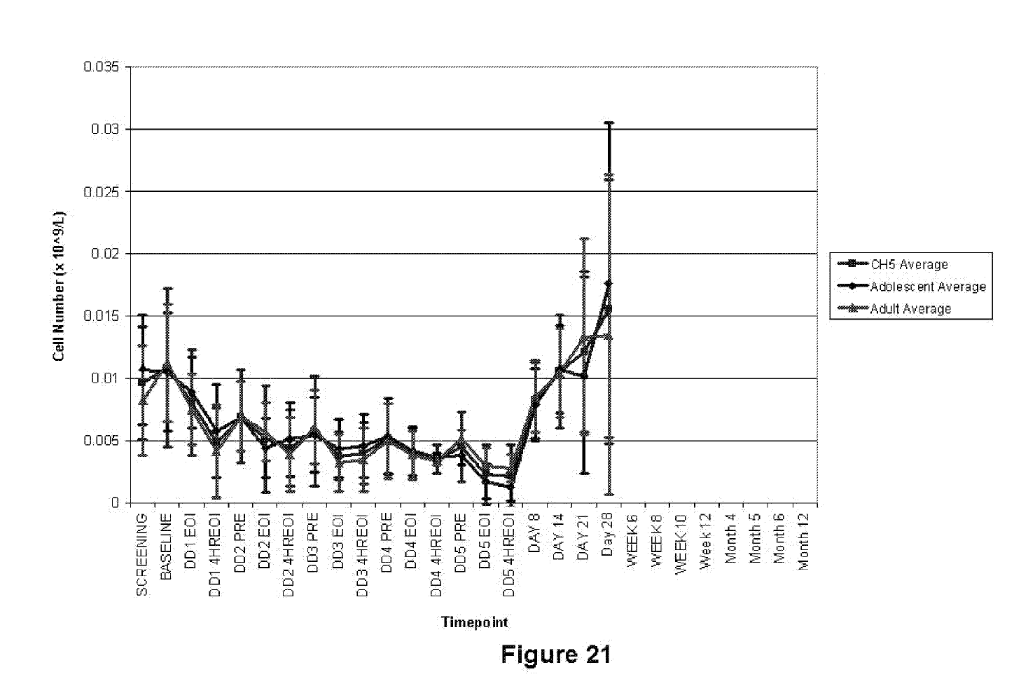

Absolute counts for Treg cells (CD4+CD25+FoxP3+) T cells were determined based on CD markers as described above. Absolute counts and percentages were calculated for each parameter, and changes from baseline were determined for each post-baseline assessment. CD4+CD25+FoxP3+ T cell results for dosing regimen CH5 are shown in FIG. 21. Data for all subjects, adolescent subjects (up to 17 or 18 years of age and younger) and adult subjects (17 or 18 years of age and older) in the CH5 study are separately shown (FIG. 21). A transient decrease in these cells was observed during dosing. The number of lymphocytes recovered to baseline levels between day 14 and day 21 (FIG. 21) shows that CD4+CD25+FoxP3+ T cells increased.

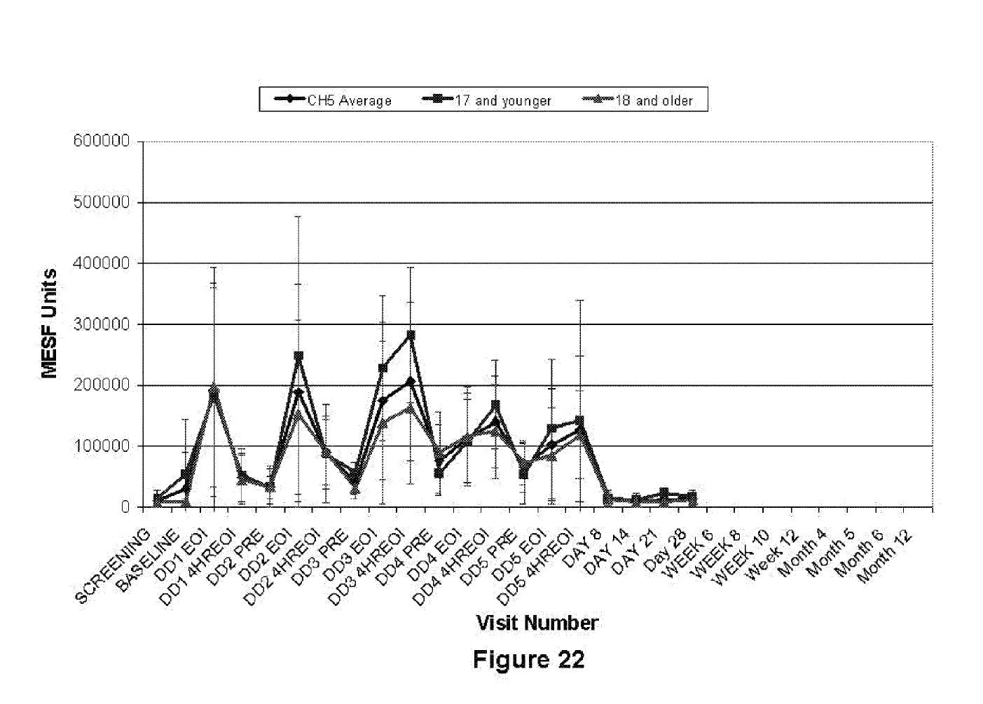

Cell-bound otelixizumab on CD4+ T cells was determined using anti-human IgG antibody reagents, and fluorescence intensity was quantified by using standard MESF units as described above (FIG. 22). Data for all the subjects, subjects 17 years of age and younger, and subjects 18 years of age and older in the CH5 study are separately shown. When the infusions of otelixizumab were stopped after Day 5, the levels CD4+ T cell bound otelixizumab returned to baseline after dosing ended.

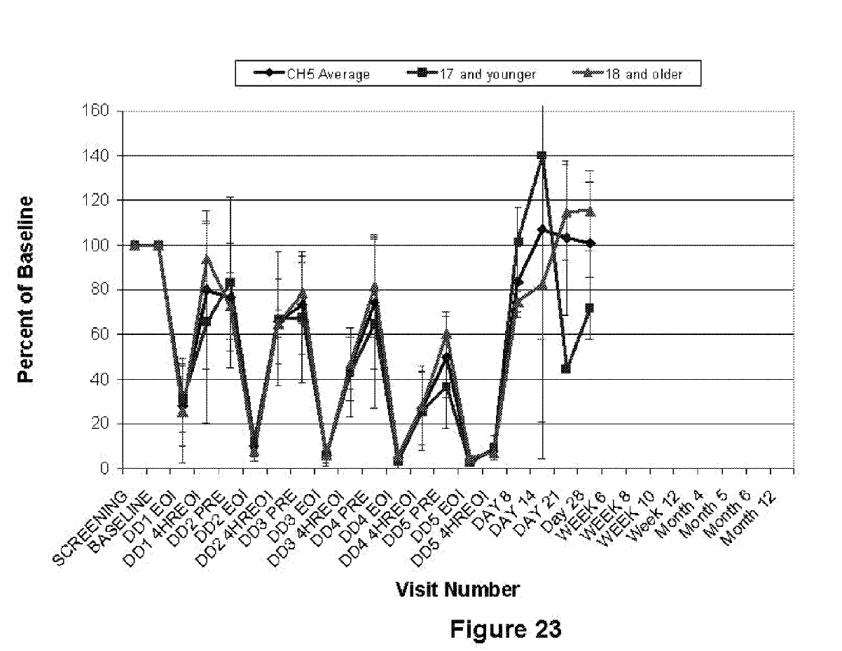

The number of CD3/TCR sites on CD4+ T cells was determined using the noncompeting anti-TCR antibody BMA031 as described above. Results for dosing regimen CH5 are calculated as the percentage of CD3/TCR sites compared to baseline (FIG. 23) Data for all the subjects, subjects 17 years of age and younger, and subjects 18 years of age and older in the CH5 study are separately shown. Subjects administered dosing regimen CH5 exhibited a transient decrease in the number of CD4+ T cell CD3/TCR sites in peripheral blood during dosing; and the number of CD4+ T cell CD3/TCR sites recovered to baseline levels after dosing ended.

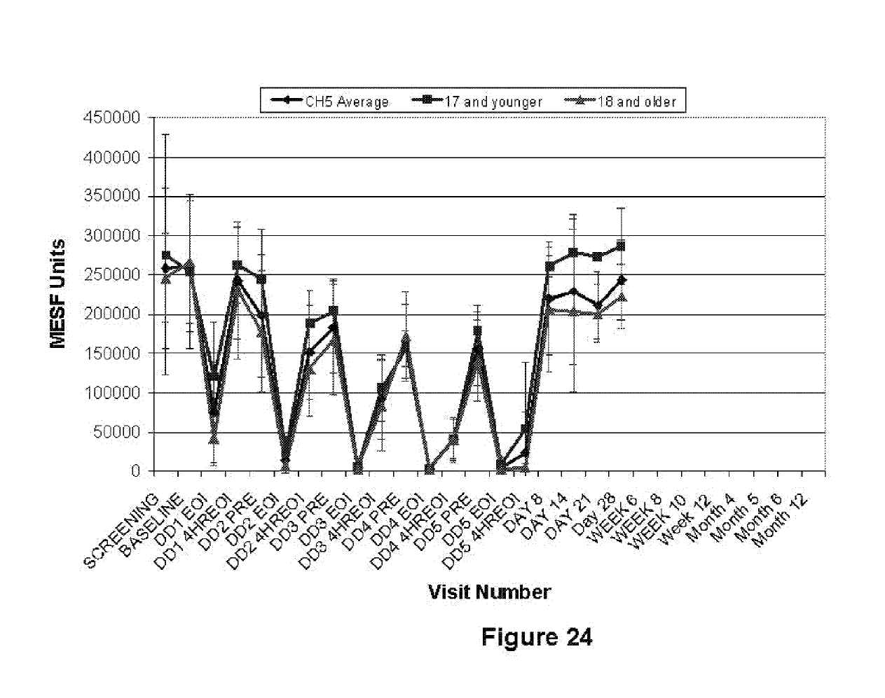

The number of free CD3 sites on CD4+ T cells was determined using biotinylated otelixizumab as described above. Results for dosing regimen CH5 are calculated in MESF units (FIG. 24). Data for all the subjects, subjects 17 years of age and younger, and subjects 18 years of age and older in the CH5 study are separately shown. Subjects administered dosing regimen CH5 exhibited a transient decrease in the number of free CD3 sites in peripheral blood during dosing; and the number of free CD3 sites recovered to baseline levels after dosing ended.

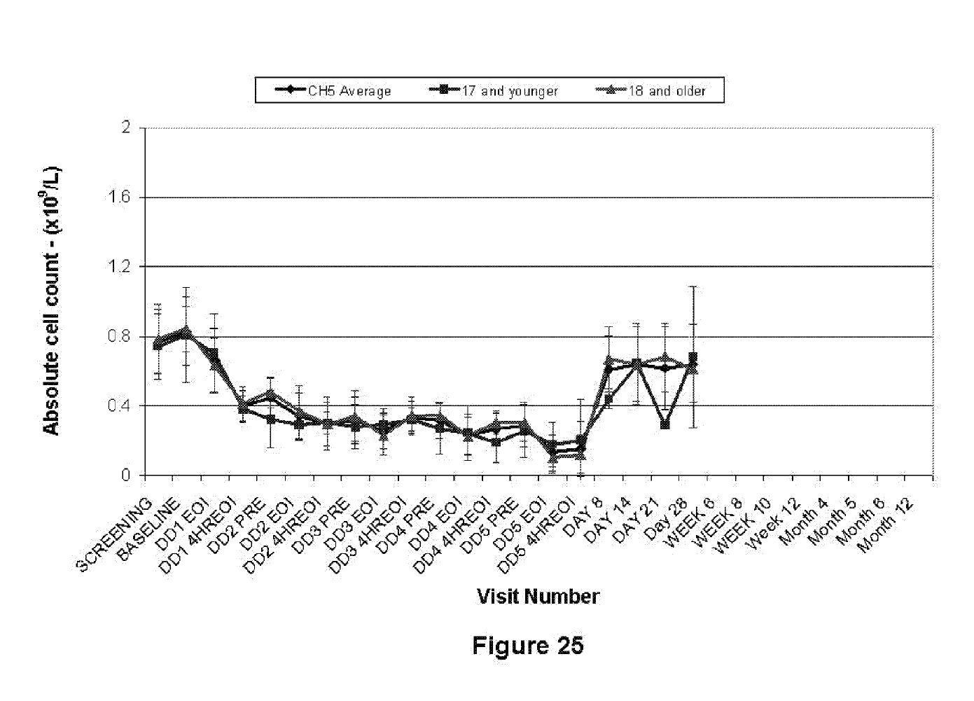

Absolute counts for CD4+ T cells were determined as described above (FIG. 25). Data for all the subjects, subjects 17 years of age and younger, and subjects 18 years of age and older in the CH5 study are separately shown. Subjects administered dosing regimen CH5 exhibited a transient decrease in the number of CD4+ T cells in peripheral blood during dosing; and the number of CD4+ T cells recovered to baseline levels after dosing ended.

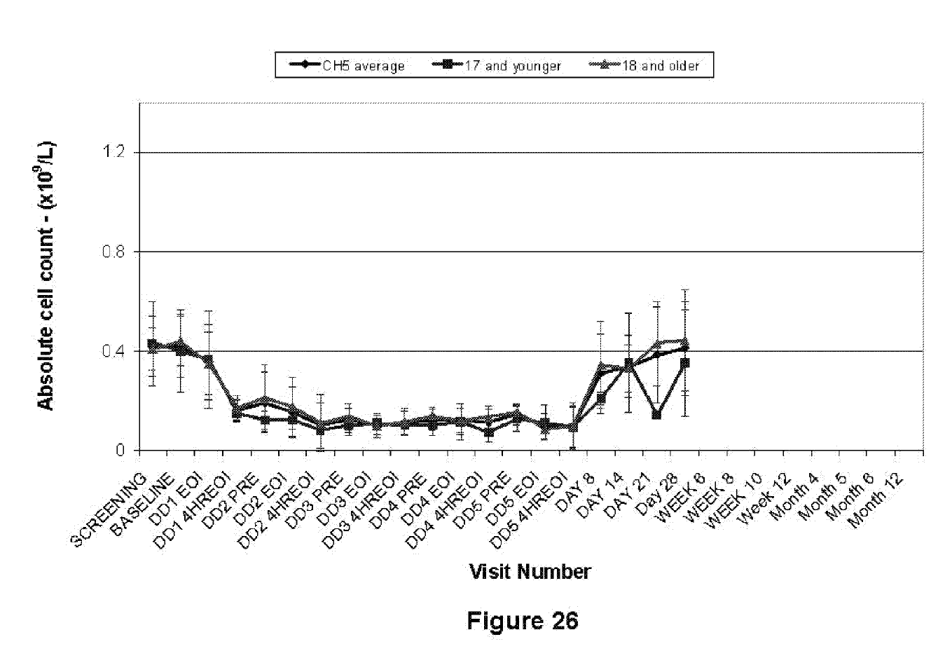

Absolute counts for CD8+ T cells were determined as described above (FIG. 26). Data for all the subjects, subjects 17 years of age and younger, and subjects 18 years of age and older in the CH5 study are separately shown. Subjects administered dosing regimen CH5 exhibited a transient decrease in the number of CD8+ T cells in peripheral blood during dosing; and the number of CD8+ T cells recovered to baseline levels after dosing ended.

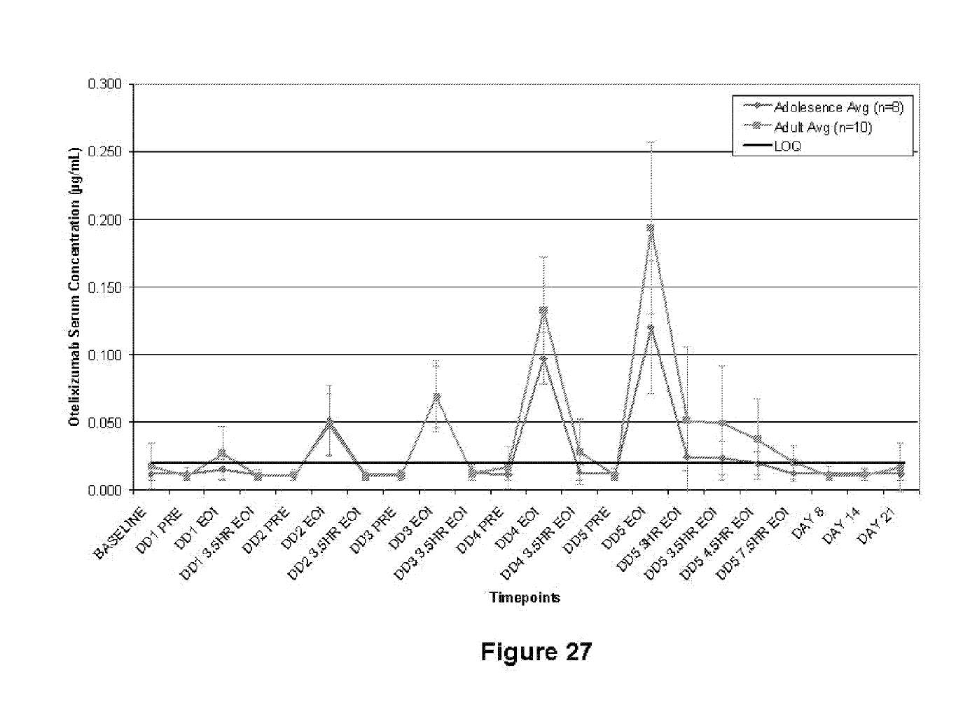

Serum levels of otelixizumab were determined by ELISA assay as described above. Data for adolescent subjects (17 years of age and younger) and adult subjects (18 years of age and older) in the CH5 study are separately shown (FIG. 27). The LOQ (limit of quantification) is shown in the figure. Serum levels of otelixizumab rose after each daily administration, and then dropped back to baseline by the next day.

Peak cytokine profiles of IL6 and TNF-alpha, perturbation of EBV immunity, and immunogenicity are determined as described above. It is expected that peak cytokine profiles of IL6 and TNF-alpha are reduced compared to the Cohort 2 dosing regimen, which consisted of three 0.5 mg doses; no perturbation of EBV immunity is observed; and patients exhibit a decreased level of immunogenicity to the otelixizumab antibody compared to the immunogenicity that would be observed if higher doses were administered on the initial days of the dosing regimen. The levels of anti-otelixizumab antibodies detected prior to the start of the CH5 study (Baseline) and on day 28 and week 12 of the study for 18 subjects are shown in Table 4.

TABLE 4
Levels of anti-otelixizumab antibody
in eighteen subjects from the CH5 study

| Subject | Baseline | Day 28 | Week 12 |
|---|---|---|---|
| 0038-5101 | ≦0.08 μg/mL | ≦0.08 μg/mL | ≦0.08 μg/mL |
| 0139-5102 | ≦0.08 μg/mL | ≦0.08 μg/mL | 0.218 μg/mL |
| 0031-5101 | ≦0.08 μg/mL | ≦0.08 μg/mL | 0.293 μg/mL |
| 0038-5102 | ≦0.08 μg/mL | 0.123 μg/mL | 1.61 μg/mL |
| 0131-5101 | ≦0.08 μg/mL | | 0.253 μg/mL |
| 0139-5103 | ≦0.08 μg/mL | 0.149 μg/mL | 0.206 μg/mL |
| 0059-5101 | ≦0.08 μg/mL | 0.221 μg/mL | 0.367 μg/mL |
| 0038-5105 | ≦0.08 μg/mL | 0.477 μg/mL | |
| 0139-5105 | ≦0.08 μg/mL | 0.560 μg/mL | |
| 0189-5102 | ≦0.08 μg/mL | 0.144 μg/mL | |
| 0199-5103 | ≦0.08 μg/mL | 0.428 μg/mL | |
| 0208-5101 | ≦0.08 μg/mL | ≦0.08 μg/mL | |
| 0199-5102 | ≦0.08 μg/mL | ≦0.08 μg/mL | |
| 0189-5104 | ≦0.08 μg/mL | 0.226 μg/mL | |
| 0031-5102 | ≦0.08 μg/mL | | |
| 0208-5102 | ≦0.08 μg/mL | | |
| 0029-5102 | ≦0.08 μg/mL | ≦0.08 μg/mL | |
| 0139-5101 | ≦0.08 μg/mL | 1.68 μg/mL | 2.21 μg/mL |

LOQ = 0.08 μg/mL

The CH5 dosing regimen resulted in partial modulation of CD3/TCR sites and regulatory T cells were induced. In addition, lymphopenia was sustained throughout the course of the dosing regimen, but rebounded to baseline levels in the weeks following the end of the regimen.

### Example 6

### Partial TCR Modulation In Vitro Results in Inhibition of MLR

This Example examines the otelixizumab dose response on inhibition of both primary and memory mixed lymphocyte responses (MLRs), as well as on CD3/TCR modulation in vitro. In order to determine the otelixizumab concentration required for MLR inhibition and CD3/TCR modulation, several in vitro assays were performed simultaneously to evaluate both primary and memory MLRs as well as TCR and CD3 modulation. The results in this Example represent the average and standard deviations from 3 separate experiments using a total of 6 normal individuals for the MLR studies and 4 normal individuals for the TCR modulation assays.

Cells:

All assays were performed on human PBL from normal individuals that were freshly isolated from heparinized blood using Ficollpaque Plus density gradients. Briefly, heparinized tubes of blood were centrifuged at 1500 rpm for 15 minutes, after which the buffy coat was collected and placed into new sterile 50 mL conical tubes. Cells were resuspended in 35 mLs of dPBS (Dulbecco's phosphate buffered saline; Gibco 14190-144) and gently inverted before underlaying cells with 10 mLs of Ficollpaque Plus (Amersham 17-1440-03). Cells were centrifuged for 30 minutes at 2000 rpm at room temperature with no brake, and then the cell layer above the Ficollpaque was carefully removed and placed into a fresh 50 mL conical tube. Following two washes with 40 mLs of dPBS and 5 minute centrifugations of 1200 rpm at 4° C., the cells were diluted in dPBS and counted using a hemacytometer and trypan blue viable cell exclusion dye.

Primary Mixed Lymphocyte Reaction:

Responder PBL were diluted to 2×10⁶/mL in complete RPMI (i.e., RPMI medium (Gibco 11875-093) supplemented with Penicillin-Streptomycin (1:100 Gibco 15140-122), non-essential amino acids (1:100 Gibco 11140-050), Sodium Pyruvate (1:100 Gibco 11360-070)) and 10% human AB serum (Cellgro 350060-C1). Stimulator PBL from an individual HLA incompatible with the donor of the responder cells were diluted to 10×10⁶/mL in plain RPMI, and 1:20 mitomycin C (Sigma M-4287) was added for 30 minutes at 37° C. The stimulator cells were washed twice in 40 mLs of unsupplemented RPMI medium and centrifuged for 5 minutes at 1200 rpm at room temperature. Finally, the stimulator cells were recounted and resuspended at 2×10⁶/mL in complete RPMI with 10% human AB serum. Otelixizumab was diluted in complete RPMI with 10% human AB serum at twice the final concentrations. Antibody dilutions were tested in triplicate. Final concentrations of otelixizumab tested were 10, 1, 0.1, 0.01, 0.001, 0.0001 and 0 μg/mL. 100 μl of otelixizumab, 50 μl of responder cells and 50 μl of stimulator cells were added to the wells of 96-well round-bottomed tissue culture plates (Corning 3799). Triplicate wells were also plated containing responder alone (50 μl of responder cells with 50 μl of media and 100 μl of otelixizumab) as controls. Plates were incubated at 37° C. with 5% CO₂ for 5 days. At various times (2, 4, 19, 24, 48, 72, 96, and 120 hours) otelixizumab antibody was washed out of the appropriate wells by tilting the plate and removing the medium from the wells with a multi-channel pipettor. The medium removed from the wells was replaced with 200 μl of fresh RPMI+10% AB serum. Therefore, cells were exposed to otelixizumab from the initiation of the cultures for 0, 2, 4, 19, 24, 48, 72, or 96 hours or the full 5 days. On day 5, 1 μCi (25 μl per well) of ³H-thymidine was added to each well in complete RPMI+10% human AB serum. Cells were labeled for 18 hours and harvested using a Packard cell Harvester. Incorporated ³H was measured by adding 50 μl of scintillation fluid (Perkin Elmer Microscint-20 6013621) to each well and counted using a Packard Topcount. Data obtained from triplicate wells were averaged and results reported as: (cpm of antibody groups)−(responder alone cpm). This assay was performed using cells from six normal individuals as responders.

T Cell Receptor Modulation and Saturation of CD3 Receptors:

PBL were diluted to 2×10⁶ cells/mL in complete RPMI (see above)+10% AB serum (Cellgro 350060-C1) and plated in 24 well tissue culture plates at 1.5 mLs per well. Otelixizumab was added to the wells at 10, 1, 0.1, 0.01, 0.001, 0.0001 and 0 μg/mL, and there were 4 wells for each antibody concentration group (one for each time point analyzed: 4, 24, 48, 72, and 96 hours and 5 days). Plates were incubated at 37° C. with 5% CO₂. After 0, 4, 24, 48, 72, and 96 hours and 5 days, aliquots of cells (1 well/time point) were harvested from each group and split between 2 different FACS tubes to analyze CD3 saturation and TCR expression. Leftover cell suspension was pooled and used for control and compensation FACS tubes. Cells were pelleted by centrifugation at 1250 rpm, 5 minutes, 4° C., prior to staining Primary antibodies were added for 30 minutes at 4° C. and included 10 μl of CD16-PE (BD 555407), 10 μl of CD19-APC (BD 555415) and either 1 μg/mL of otelixizumab-FITC (Batch 03, 2.3 mg/mL stock) or 10 μl of TCR-FITC (Catalog MHAB01-4). Control wells received 1 μg/mL of Human IgG-FITC (Jackson Immunoresearch 009-090-003) or 1 μl/test of mouse IgG2b-FITC (Ancell 284-040) respectively. After staining, cells were washed twice in FACS buffer (DPBS (Gibco 14180) with 0.2% FBS (Hyclone SH30071.03) and 0.1% sodium azide (VWR VW3465-2)) by resuspending cells with 2 mLs of FACS buffer and centrifuging them at 1200 rpm for 5 minutes at 4° C. Finally, the cells were resuspended in 200 μl of FACS buffer for analysis. This assay was performed on cells from four normal individuals.

Memory Mixed Lymphocyte Reaction:

Responder PBL were diluted to 2×10⁶/mL in complete RPMI (RPMI media (Gibco 11875-093) with Penicillin-Streptomycin (1:100 Gibco 15140-122), non-essential amino acids (1:100 Gibco 11140-050), Sodium Pyruvate (1:100 Gibco 11360-070)) and 10% human AB serum (Cellgro 350060-C1). Stimulators PBL from an individual HLA incompatible with the donor of the responder cells were diluted to 10×10⁶/mL in plain RPMI, and 1:20 mitomycin C (Sigma M-4287) was added for 30 minutes at 37° C. The stimulator cells were washed twice in 40 mLs of plain RPMI and centrifuged for 5 minutes at 1200 rpm at room temperature. The stimulator cells were recounted and resuspended at 2×10⁶/mL in complete RPMI with 10% human AB serum. Stimulators and responders were then cultured in T75 tissue culture flasks (BD Falcon 137787) at a 1:1 ratio for 7 days. After 7 days, cultures were harvested, washed, recounted and resuspended at 2×10⁶/mL in complete RPMI with 10% human AB serum. This harvested cells were used as responder cells in the memory MLR reaction. Fresh stimulators were prepared from the original donor as well as an unrelated donor as described above and resuspended at 2×10⁶/mL in complete RPMI with 10% human AB serum. Otelixizumab was diluted in complete RPMI with 10% human AB serum at twice the final concentrations. Antibody dilutions were tested in triplicate. Final concentrations of otelixizumab tested were 1, 0.5, 0.1, 0.05, 0.01, 0.005, 0.001 and 0 μg/mL. 100 μl of otelixizumab, 50 μl of responder cells and 50 μl of stimulator cells were added to wells of a 96-well round bottom plate (Corning 3799). Triplicate wells were also plated containing responder cells alone (50 μl of responder cells with 50 μl of media and 100 μl of otelixizumab) as controls. Plates were incubated at 37° C., 5% CO₂ for 3 days. On day 3, 1 μCI (25 μl per well) of ³H-thymidine was added to each well in complete RPMI+10% human AB serum. Cells were labeled for 18 hours and harvested using a Packard cell Harvester. Incorporated ³H was measured by adding 50 μl of scintillation fluid (Perkin Elmer Microscint-20 6013621) to each well and counted using a Packard Topcount. Data from triplicate wells were averaged and results reported as: (cpm of antibody groups)−(responder alone cpm). This assay was performed using cells from six normal individuals as responders.

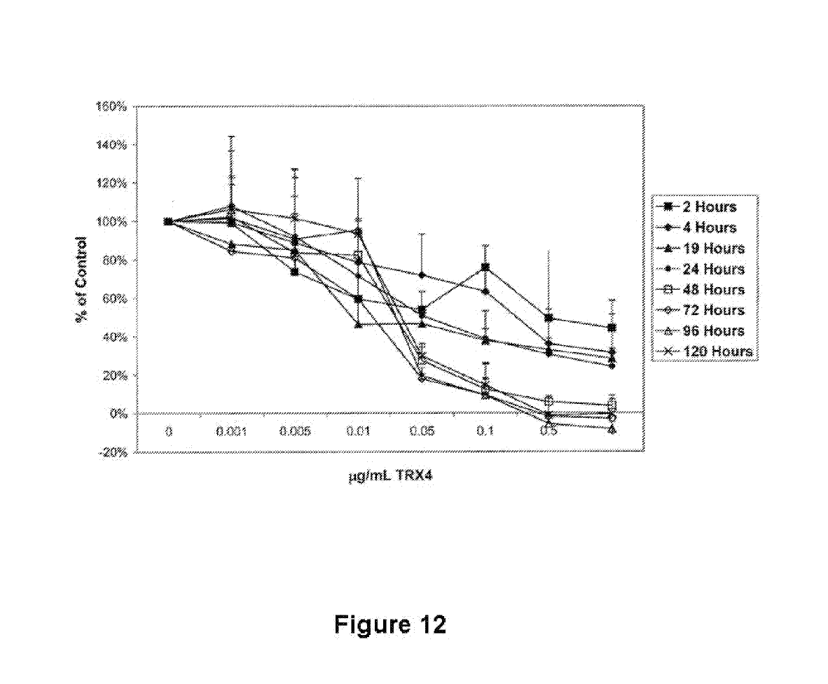

Otelixizumab inhibited the primary MLR in culture medium supplemented with human AB serum at levels greater than 85% when PBL was exposed to at least 0.1 μg/mL of otelixizumab for at least 48 hours (FIG. 12). When cells were incubated for less than 48 hours in the presence of 0.1 μg/mL of otelixizumab inhibition ranged from 24-61%, whereas 48 hours or more of exposure to otelixizumab resulted in inhibition ranging from 85-91%. Maximal inhibition occurred at concentrations greater than or equal to 0.5 μg/mL when cells were incubated for at least 48 hours.

Binding of anti-CD3 antibodies to T cells results in temporary modulation of the CD3/TCR complex from the surface of the T cell. Assays assessing the ability of otelixizumab to modulate the CD3/TCR complex were performed on PBL from 4 normal individuals in the presence of human AB serum. Modulation was determined by monitoring the presence of the CD3/TCR complex by 2 methods: free otelixizumab binding sites were monitored with exogenous otelixizumab-FITC; and the presence of the CD3/TCR complex on the cell surface was monitored with a non-competing anti-TCR antibody.

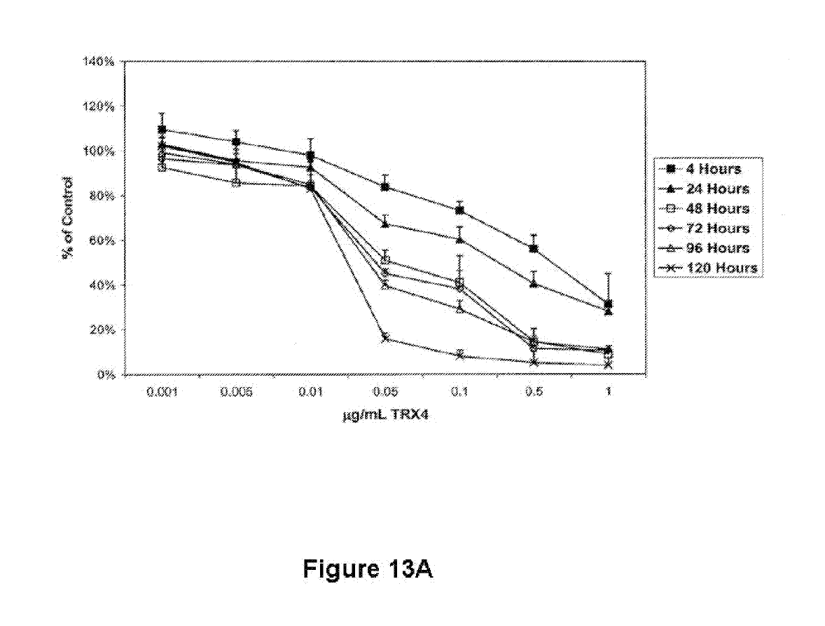

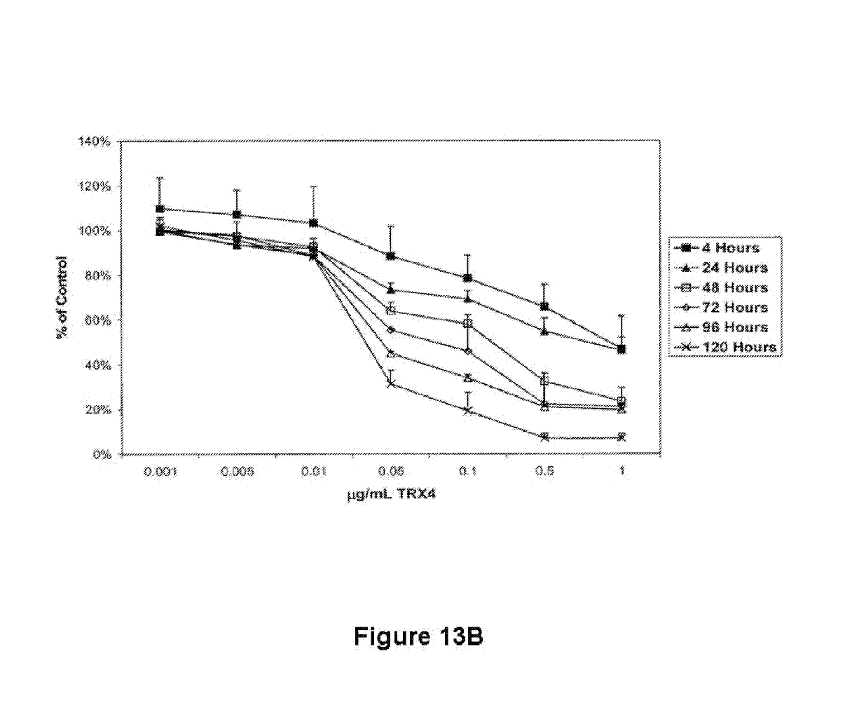

Otelixizumab significantly decreased the number of free CD3 sites when normal PBL were exposed to at least 0.1 μg/mL of otelixizumab for greater than 48 hours (FIG. 13A). Whereas less than 24 hours of 0.1 μg/mL of otelixizumab exposure decreased free CD3 sites by 27-40%, 48 hours or more of exposure decreased free CD3 sites by 59-92%. Maximum saturation of CD3 sites was seen with exposure of at least 0.5 μg/mL for 120 hours. Similarly, otelixizumab concentrations of 0.1 μg/mL or more for at least 48 hours significantly decreased expression of the TCR (FIG. 13B). Less than 24 hours exposure to 0.1 μg/mL of otelixizumab resulted in a decrease in TCR expression of 21-31%, whereas 48 hours or more of exposure to 0.1 μg/mL caused a decrease in TCR expression of 42-81%. Maximum modulation of the TCR was seen with exposure of 0.5 μg/mL or greater of otelixizumab for 120 hours.

Direct comparison of primary MLR inhibition with TCR modulation data shows that 5 days of otelixizumab exposure resulted in similar inhibition of both of these parameters with a concentration of at least 0.01 μg/mL of otelixizumab (Table 5). TCR expression showed 88% of control levels with 120 hours exposure to 0.01 μg/mL of otelixizumab, 31% of control levels with 0.05 μg/mL, and 19% with 0.1 μg/mL. Primary MLR responses show 94%, 30%, and 14% of the control value at antibody concentrations of 0.01, 0.05 and 0.1 μg/mL respectively. This similarity indicates that the pharmacodynamic parameter of TCR expression has a close correlation (correlation coefficient r=0.999) with the in vitro functional parameter of primary MLR inhibition.

TABLE 5
Pharmacodynamic Parameter Studies - Comparison of 120 Hours of
Otelixizumab Exposure

| ug/ml | MLR - | | Free CD3 sites - |
|---|---|---|---|
| otelixizumab | % of control | TCR - % of control | % of control |
| 0.001 | 106.3 | 102 | 102 |
| 0.005 | 101.5 | 96 | 95 |
| 0.01 | 93.6 | 88 | 83 |
| 0.05 | 29.7 | 31 | 16 |
| 0.1 | 14.4 | 19 | 8 |
| 0.5 | −1.1 | 7 | 5 |
| 1 | −0.4 | 7 | 4 |

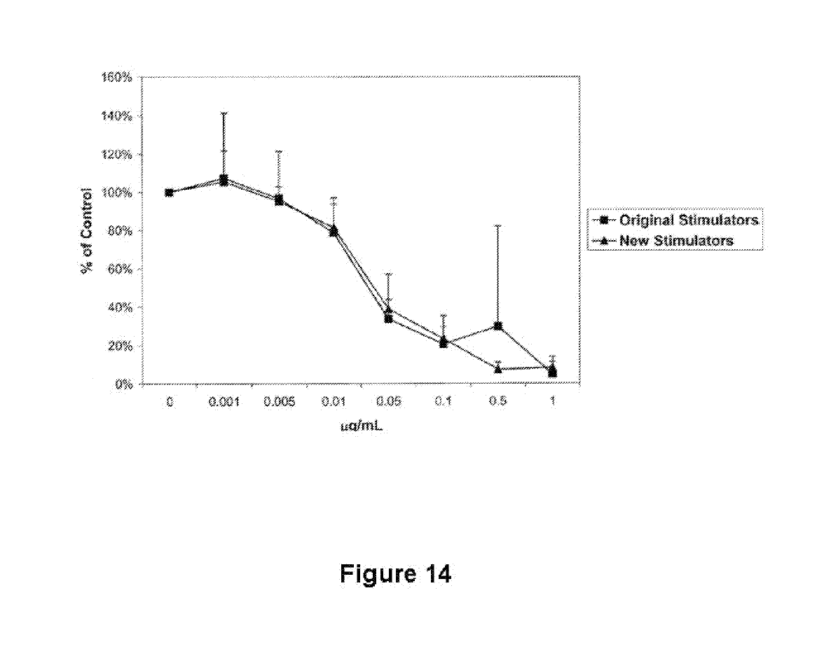

To determine whether otelixizumab inhibits memory MLR responses, responder cells were cultured in the presence of stimulators without otelixizumab for 7 days. After washing, cells were re-stimulated with either the original stimulator or a new (novel) stimulator in the presence of various concentrations of otelixizumab for 3 days. Otelixizumab inhibited the memory MLR response (using stimulator PBL from the same individual that the stimulator PBL for the initial 7 day culture were obtained from) comparably to that of a primary MLR response (using stimulator PBL from a different individual than that which the stimulator PBL for the initial 7 day culture were obtained from) (FIG. 14). Inhibition was seen starting at 0.01 μg/mL of otelixizumab and resulted in an inhibition of 19-21% for memory (restimulation with the original stimulators) or primary (stimulation with a novel stimulator) responses. A marked increase in inhibition was seen when 0.05 μg/mL of otelixizumab was used (61-66%), and maximum inhibition was seen with 1 μg/mL or higher.

This Example demonstrates that exposure to at least 0.1 μg/mL of otelixizumab for 48 hours or more showed marked reductions in TCR expression, free CD3 sites, and inhibition of primary and memory MLR responses. This Example indicates that downregulation of CD3/TCR expression and inhibition of a primary MLR response show good correlation in response to a broad range of otelixizumab concentrations (0.001-1.0 μg/mL) when cells are exposed to otelixizumab for 5 days. Finally, the data suggest that otelixizumab can comparably inhibit primary and memory MLR responses. In summary, in vitro TCR expression correlates with otelixizumab dose and that TCR expression may be monitored to gauge in vivo efficacy of anti-CD3 antibody treatment.

### Example 7

### Low Dose Regimens of Anti-CD3 mAb Induce Remission in NOD Mice

Preclinical and clinical experience supports the rationale for treatment of patients with new-onset autoimmune type 1 diabetes with anti-CD3 monoclonal antibodies (mAbs) and fragments (e.g., F(ab′)₂ fragments) thereof. In a Phase 2 trial conducted by the Belgian Diabetes Registry (BDR), subjects with new-onset type 1 diabetes who received a single 6-day course of otelixizumab (total dose 48-64 mg) had significantly greater endogenous insulin production than subjects who received placebo, and this effect was durable for at least 48 months.

Upon anti-CD3 mAb administration, antibody rapidly binds the CD3 molecule and is internalized, resulting in modulation of the CD3/TCR-complex. Loss of CD3/TCR-complex expression is reversible, as it recycles back to the surface after clearance of the antibody. Binding and subsequent modulation of the CD3/TCR-complex by anti-CD3 mAb are considered pharmacodynamically important and should be assessed in clinical studies evaluating anti-CD3 mAb therapies. This pharmacodynamic (PD) effect potentially impacts the mechanism of action of anti-CD3 mAb in at least 2 ways: (1) temporarily blocking antigen binding and (2) delivering a partial agonist signal, which may induce anergy of autoreactive T-cells while allowing for the expansion of Treg cells.

In this Example, dose-ranging studies in diabetic NOD mice were performed to determine the minimum effective dose of anti-CD3 mAb F(ab′)₂. CD3/TCR-complex modulation patterns elicited during antibody administration were assessed to determine whether nearly complete and sustained modulation is required for efficacy of anti-CD3 mAb F(ab′)₂ therapy. Doses resulting in partial and transient CD3/TCR-complex modulation were sufficient to induce remission in diabetic NOD mice, such that doses more than 30-fold less than the originally published 250 μg regimen resulted in similar rates of remission. PD effects on lymphocyte counts and circulating T-cell subsets were also measured, demonstrating that the efficacy of anti-CD3 mAb F(ab′)₂ treatment is associated not only with PD changes anticipated based on the mAb's mechanism of action, but also with residual beta-cell function at the time of treatment.

Mice:

BALB/c mice (Harlan, Boston, Mass.) were used in Study A. Female NOD/ShiLtJ mice (Jackson, Bar Harbor, Me.) were used for Study B; NOD/ShiLtJ mice were bred at Tolerx under pathogen-free conditions for Study C.

Antibodies:

Hamster anti-mouse CD3 mAb (clone 145-2C11; ATCC) was purified using protein G affinity chromatography (GE Healthcare, Piscataway, N.J.) and formulated in Dulbecco's PBS. Anti-CD3 mAb F(ab′)₂ fragments were generated by pepsin (Sigma, St. Louis, Mo.) digestion for 17 hr at 37° C. in acetic acid, pH 4.0. The reaction was quenched with 2 M Tris and dialyzed against PBS overnight at 2-8° C. F(ab′)₂ fragments were further purified by size-exclusion chromatography. Purity was assessed by SDS-PAGE (sodium dodecyl sulfate polyacrylamide gel electrophoresis) and found to be 90% of total integrated density with no intact antibody. The F(ab′)₂ preparation included ≦3 endotoxin units/mL, as measured by Pyrotell gel-clot assay (Associates of Cape Cod, East Falmouth, Mass.),

Anti-CD3 mAb F(ab′)₂ Treatment:

In Study A, BALB/c mice were dosed with the following regimens: 5 doses of 50 μg every 24 hr (total dose 250 μg); 4 doses of 25 μg every 72 hr (total dose 100 μg); 4 doses of 5 μg every 72 hr (total dose 20 μg); 4 doses of 2 μg every 72 hr (total dose 8 μg); and 4 doses of 1 μg every 72 hr (total dose 4 μg). In Study B, NOD/ShiLtJ mice were administered the following dose regimens: 5 doses of 50 μg every 24 hr (total dose 250 μg); 4 doses of 25 μg every 72 hr (total dose 100 μg); 3 doses of 25 μg every 72 hr (total dose 75 μg); 4 doses of 5 μg every 72 hr (total dose 20 μg); and 3 doses of 5 μg every 72 hr (total dose 15 g). In Study C, NOD/ShiLtJ mice were administered the following dose regimens: 3 doses of 5 μg every 72 hr (total dose 15 μg); 4 doses of 2 μg every 72 hr (total dose 8 μg); and 4 doses of 1 μg every 72 hr (total dose 4 μg). Each study also included a vehicle (PBS) control. All doses were delivered i.p. In Studies B and C, blood glucose levels were measured twice weekly in female NOD/ShiLtJ mice. Mice with 2 consecutive blood glucose levels >250 mg/dL were considered to have new-onset diabetes and enrolled in the study such that variation in age at disease onset was equally represented across dose regimens. After treatment, blood glucose was measured weekly. Remission was defined as a return to normal glycemia in the absence of exogenous insulin.

Immunogenicity Assay:

An ELISA-based assay was developed to determine whether an immunogenic response towards the anti-CD3 mAb F(ab′)₂ had been induced in anti-CD3 mAb-F(ab′)-2-treated mice. Maxisorp 98 well plates (Nunc, Rochester, N.Y.) were coated with anti-CD3 mAb F(ab′)₂. Following incubation with mouse serum from treated mice, mouse antibodies specific for anti-CD3 mAb F(ab′)₂ were detected with donkey anti-mouse IgG (H+L) (HRP-conjugated, minimally cross-reactive to hamster species; Jackson ImmunoResearch, West Grove, Pa.). ELISAs were developed using O-phenyl diamine dihydrochloride (OPD) substrate (Sigma) in sodium citrate buffer pH 5 plus H₂O₂. 12.5% H₂SO₄ was used to stop the OPD reaction, and plates were read at 490 nm using Softmax™ Pro software (MDS Analytical Technologies, Sunnyvale, Calif.).

Flow Cytometry Analysis:

CD3/TCR-complex modulation in peripheral blood was analyzed by flow cytometry 2 hr and 24 hr dose. Following red blood cell lysis, cells were stained using murine antibodies to CD3 (145-2C11), CD4 (RM4-5), CD8 (53-6.7), and TCR-beta (H57-597) (BD Biosciences, San Jose, Calif.). Molecules of Equivalent Soluble Fluorochrome (MESF) values were generated using Quantam™ FITC MESF microspheres per manufacturer's directions (Bangs Laboratories, Fisher, Ind.). FoxP3 expression was evaluated using a FoxP3 staining kit (NRRF30 clone; EBioscience, San Diego, Calif.) per manufacturer's directions. Fluorescent cells were analyzed by flow cytometry using a FACScaliber flow cytometer (BD Biosciences).

Analysis of C-Peptide Levels in Serum:

In Study B, serum was collected before and after treatment and analyzed for murine C-peptide content by ELISA per manufacturer's instructions (ALPCO, Salem, N.H.).

Pancreatic Histology:

In Study B, pancreata were fixed in formalin, processed, and embedded in paraffin. Four- to five-μm sections were stained with hematoxylin and eosin. Islet inflammation was evaluated with light microscopy by a board-certified veterinary pathologist (Charles River Laboratories, Wilmington, Mass.). Peri-insulitis inflammation was scored as: 0=normal (no leukocytes); 1=minimal (<5 leukocytes in any islet); 2=mild (6-20 leukocytes in “most severe” islet); 3=moderate (21-50 leukocytes in “most severe” islet); 4=marked (>50 leukocytes in “most severe” islet); or 5=severe (>50 leukocytes in >1 islet).

Statistical Methods:

MESF values were analyzed using repeated-measures analysis of variance (ANOVA) with treatment and time as factors. Lymphocyte count data were analyzed by one-way ANOVA. Pairwise treatment group comparisons for these analyses were carried out using the corresponding t-tests. Fisher's exact test was used for pairwise treatment group comparisons of proportion data. Exploratory comparisons between post-treatment remission and diabetic groups were made by t-test (quantitative data), Fisher's exact test (proportion data), or chi-square test (categorical data). P-values were not adjusted for multiple comparisons.

Modified Dose Regimens Result in Transient and Partial CD3/TCR-Complex Modulation:

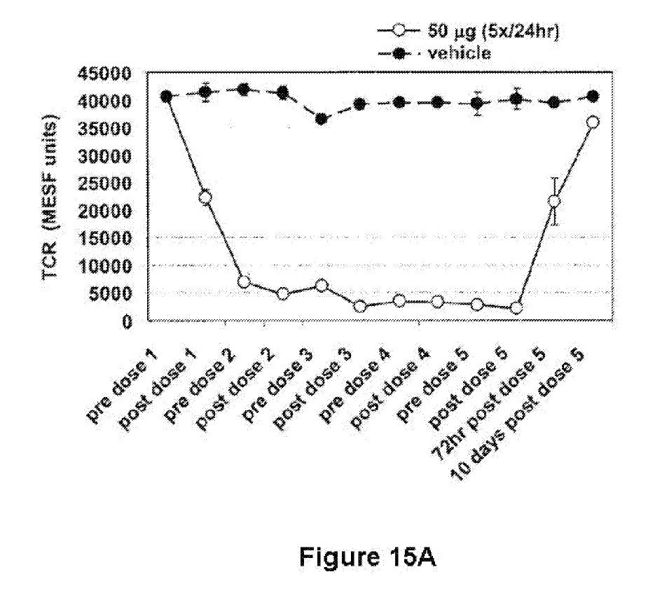

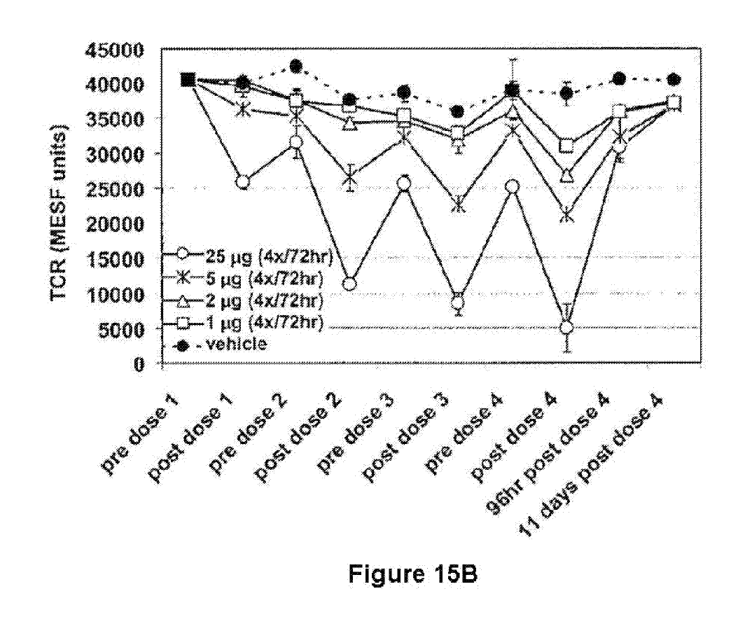

In previous preclinical studies, a 250 μg anti-CD3 mAb F(ab′)₂ dose regimen, 50 μg per day for 5 consecutive days (50 μg [5×/24 hr]), resulted in a 67% remission rate in new-onset diabetic NOD mice (Chatenoud, L., Primo, J. & Bach, J. F. CD3 antibody-induced dominant self tolerance in overtly diabetic NOD mice. J Immunol 1997; 158: 2947-54, incorporated herein by reference in its entirety), but there are limited data evaluating PD effects during dosing. Given that it has been previously demonstrated that the biological effects of the antibody are similar in NOD and non-autoimmune mice, the PD effects of anti-CD3 mAb F(ab′)₂ on CD3/TCR-complex modulation in BALB/c mice in Study A were first examined. TCR expression on peripheral blood CD4+ and CD8+ lymphocytes was analyzed 2 hr and 24 hr after each dose. The resulting patterns of TCR expression on both CD4+ and CD8+ lymphocytes were equivalent; therefore, only CD4+ lymphocytes are shown in FIG. 15. In the first segment, the well-established 50 μg (5×/24 hr) anti-CD3 mAb F(ab′)₂ dose regimen was evaluated. CD3/TCR-complex expression was reduced 2 hr after the first dose and remained almost completely down-regulated prior to the second dose. These low levels of CD3/TCR-complex expression were sustained throughout dosing (FIG. 15A), similar to the pattern observed in the BDR clinical trial where high dose regimens of otelixizumab were evaluated. See Keymeulen, B., Vandemeulebroucke, E., Ziegler, A. G. et al. Insulin needs after CD3-antibody therapy in new-onset type 1 diabetes. N Engl J Med 2005; 352: 2598-608, incorporated herein by reference in its entirety. CD3/TCR-complex expression was partially restored within 72 hr following the end of dosing and returned to baseline within 10 days of the last dose.

Since the 50 μg (5×/24 hr) dose regimen resulted in nearly complete and sustained CD3/TCR-complex modulation, the development and evaluation of dose regimens that would elicit a partial and transient pattern of modulation was of interest. First, lower doses of anti-CD3 mAb F(ab′)₂ were evaluated. TCR expression was measured in BALB/c mice administered 5 doses of 25, 5, 2, or 1 μg anti-CD3 mAb F(ab′)₂, 24 hr apart. The 25 μg (5×/24 hr) dose regimen resulted in profound and sustained CD3/TCR-complex modulation, similar to the 50 μg (5×/24 hr) regimen. Lower doses produced dose-dependent reductions in CD3/TCR-complex modulation, but a sustained level of modulation was observed in all dose regimens. This suggested that spacing the doses further apart may achieve a pattern of transient CD3/TCR-complex modulation. It was next determined how soon after dosing the surface expression of CD3/TCR complex returns to baseline levels in the mouse. After a single 25 μg dose of anti-CD3 mAb F(ab′)₂, CD3/TCR-complex expression was markedly down-regulated at 24 h; beginning to recover, but still significantly down-regulated at 48 h; and recovered to near baseline values at 72 hr.

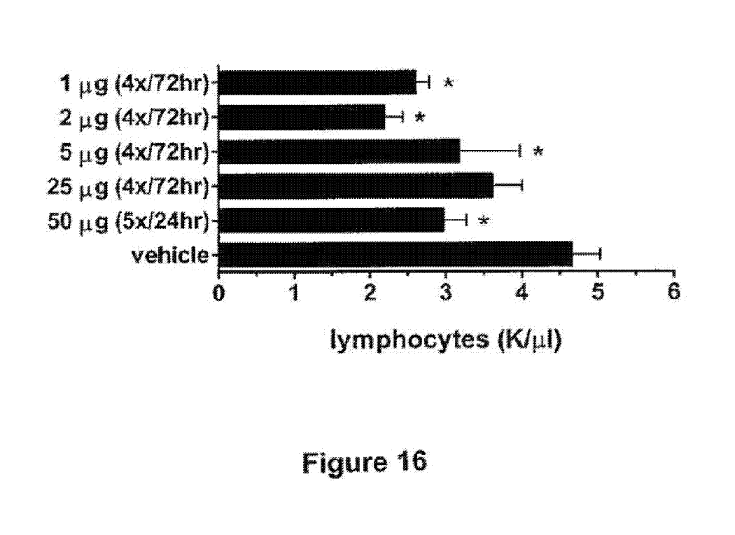

In the second segment of Study A, a range of anti-CD3 mAb F(ab′)₂ doses (1, 2, 5, and 25 μg) was administered 4 times, 72 hr apart, given that a fifth dose resulted in anti-drug antibodies in 3 out of 6 mice (detected using an ELISA-based assay). The mice did not develop any adverse events associated with immunogenicity to the anti-CD3 mAb F(ab′)₂. The 72-hr dose regimen resulted in transient and sometimes partial CD3/TCR-complex modulation that was clearly dose-dependent (FIG. 15B). The 5 and 25 μg (4×/72 hr) dose regimens produced “saw-tooth” patterns, where CD3/TCR-complex expression was quickly down-regulated after each dose but returned to near pre-dose values before the subsequent dose. With each successive dose, the level of CD3/TCR-complex modulation increased. In the 2 μg and 1 μg (4×/72 hr) dose regimens, the extent of modulation was considerably less than other dose regimens and clearly discernable only after the fourth dose (FIG. 15B). After the fourth dose, the difference in the percentage of CD3/TCR-complex modulation between the 2 μg and 1 μg (4×/72 hr) dose regimens was significant (30.3% vs. 19.7% modulation, p<0.01). Furthermore, in all dose regimens, there was a transient decrease in lymphocyte numbers in the peripheral blood during and shortly after dosing (FIG. 16), consistent with what has been observed in the spleens of both NOD and non-autoimmune mice administered anti-CD3 mAb F(ab′)₂. This observation of lymphopenia during dosing could be the result of either depletion of a subset of lymphocytes or re-trafficking of anti-CD3 mAb F(ab′)₂-bound lymphocytes from the peripheral blood.

Lower Doses of Anti-CD3 mAb F(ab′)₂ are Efficacious in New-Onset Diabetic NOD Mice:

In Study B, the effectiveness of the various dose regimens in inducing remission of diabetes was investigated in new-onset diabetic NOD mice. In order to evaluate whether a shorter duration of CD3/TCR-complex modulation or a lower cumulative dose affects efficacy, Study B also included groups given only 3 doses. Animals were randomly enrolled into one of 5 anti-CD3 mAb F(ab′)₂ dose regimens: 50 μg (5×/24 hr), 25 μg (4×/72 hr), 25 μg (3×/72 hr), 5 μg (4×/72 hr), or 5 μg (3×/72 hr), or placebo. The 25 μg and 5 μg doses were chosen based on the results of Study A, in which CD3/TCR-complex expression 24 hr after dose 4 was approximately 12% and 50% of baseline, respectively. No animals in the placebo group entered remission during the 12-week observation of blood glucose levels. In all dose regimens, approximately half the mice (44% to 60%) had long-term remission (Table 6). There was no statistically significant difference in remission rates between the various dose regimens. The well-established 50 μg (5×/24 hr) dose regimen resulted in 56% of the mice being in remission for 12 weeks, which is similar to the originally published 67% remission rate. There was no apparent relationship between dose and rate of remission. As in previous studies, the majority of mice in all dose regimens that entered remission did so 1-2 weeks after treatment and all remained in remission for the 12 weeks of follow-up.

TABLE 6
Remission Rates of New-onset Diabetic Female NOD/ShiLtJ Mice Treated with
Anti-CD3 mAb F(ab′)2 Fragments

| | | | | |
|---|---|---|---|---|
| | | | Percent | |
| | | Total dose | (number) | |
| | | as a multiple | experiencing | Duration of |
| Dose regimen | Total dose | of 250 μg | remission | remission |

Study B

| | | | | |
|---|---|---|---|---|
| 50 μg × 5/24 hr (250 μg) | 250 μg (0.0125 mg/kg) | 1 | 56% (9/16) | 12 weeks^{§} |
| 25 μg × 4/72 hr (100 μg) | 100 μg (0.005 mg/kg) | 1/2.5 | 50% (8/16) | 12 weeks^{§} |
| 25 μg × 3/72 hr (75 μg) | 75 μg (0.0038 mg/kg) | 1/3.3 | 50% (8/16) | 12 weeks^{§} |
| 5 μg × 4/72 hr (20 μg) | 20 μg (0.001 mg/kg) | 1/12.5 | 60% (9/15) | 12 weeks^{§} |
| 5 μg × 3/72 hr (15 μg) | 15 μg (0.00075 mg/kg) | 1/16.6 | 44% (7/16) | 12 weeks^{§} |
| Placebo | — | — | 0% (0/15) | N/A |

Study C

| | | | | |
|---|---|---|---|---|
| 5 μg × 3/72 hr (15 μg) | 15 μg (0.00075 mg/kg) | 1/16.6 | 63% (5/8) | 12 weeks |
| 2 μg × 4/72 hr (8 μg) | 8 μg (0.0004 mg/kg) | 1/31.3 | 53% (10/19)* | 12-24 weeks |
| 1 μg × 4/72 hr (4 μg) | 4 μg (0.0002 mg/kg) | 1/62.5 | 16% (3/19)* | 12-24 weeks |
| Placebo | — | — | 0% (0/13) | N/A |

^{§}After 12 weeks of remission, mice were sacrificed for histological analysis.
In Study B, mice were sacrificed 12 weeks after treatment. In Studies B and C, remission rates were determined 12 weeks post-treatment. In Study C, mice in remission were not sacrificed and the range of durability was assessed up to 24 weeks post-treatment for the 2 and 1 μg dose regimens. Differences in remission rates between the 50, 25, 5, and 2 μg dose regimens in both Studies B and C were not significant, however the difference between the 2 and 1 μg dose regimens was significant (* p < 0.05)

Study B demonstrated that a total dose as low as 15 μg resulted in long-term remission of diabetes in NOD mice. In Study C, lower doses were examined to determine the minimum effective dose with the 72-hr dose regimen. Also, antibody-treated mice in Study C were followed for at least 12 weeks after treatment to determine the durability of remission and up to 24 weeks after treatment in the lowest dose regimens. The lowest dose regimen from Study B, 5 μg (3×/72 hr), was repeated, and 2 lower dose regimens, 2 μg (4×/72 hr) and 1 μg (4×/72 hr), were added. The 5 μg (3×/72 hr) and 2 μg (4×/72 hr) dose regimens, had remission rates of 63% and 53%, respectively, similar to the higher dose regimens in Study B. Again, there was no statistically significant difference in remission rates between the 5 μg (3×/72 hr) and 2 μg (4×/72 hr) dose regimens in study C or the various dose regimens in Study B. As in the higher dose regimens in Study B, these mice entered remission 1-2 weeks after treatment and the remission was long-lasting, up to the 24 weeks of follow-up. However, at the 1 μg (4×/72 hr) dose regimen, the remission rate dropped to 16% and this reduction was significantly different compared to the 2 μg (4×/72 hr) dose regimen (p<0.05). Yet, for mice that did enter remission, the remission was long-term (up to 24 weeks). Thus, the minimum effective dose of anti-CD3 mAb F(ab′)2 for the 4×/72-hr dose regimen is greater than 1 μg.

In both Studies B and C, partial remission was observed in 1 or 2 mice within each dose regimen, such that normal glycemia was detected in these mice for a transient period ranging anywhere from 3-11 weeks post-treatment. Thereafter, blood glucose levels quickly escalated and were sustained at levels greater than 250 mg/dL. There was no correlation between dose and the numbers of mice exhibiting partial remission. Overall, all of the mice that entered remission did so within 1-2 weeks after treatment consistent with previous studies, and the majority of remissions observed were durable for at least the 12-week observation period.

Treatment with Anti-CD3 mAb F(ab′)₂ Alters Proportions of T-Cell Subsets:

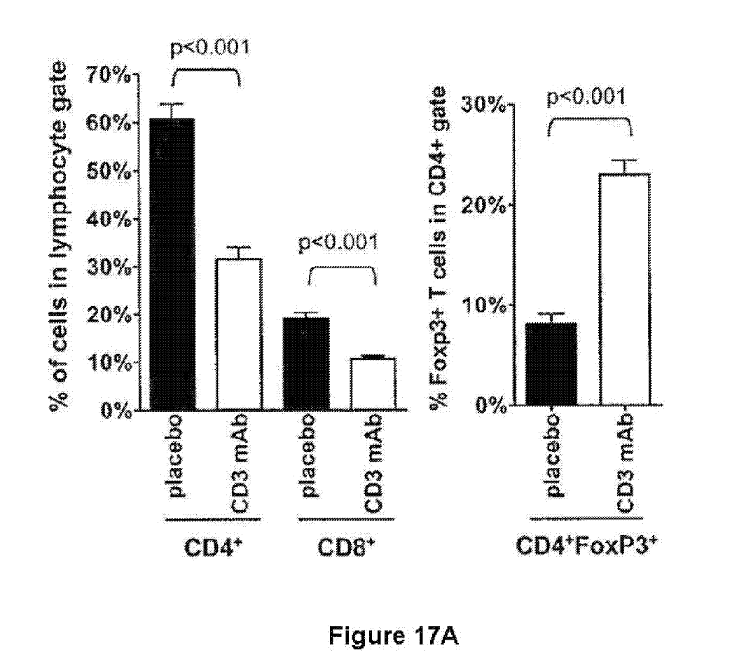

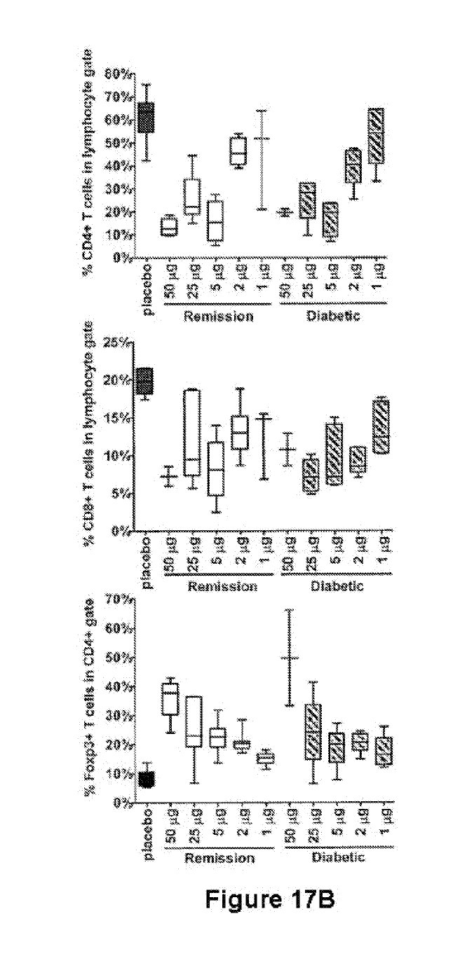

In addition to CD3/TCR-complex modulation, PD parameters often assessed in clinical studies of otelixizumab include changes in various immune cell subsets such as CD4+, CD8+, and CD4+FoxP3+ T-cells. To mirror the PD parameters routinely collected in clinical situations, similar flow cytometric PD parameters in the peripheral blood of mice from Studies B and C were evaluated. In Studies B and C, proportions of CD4+, CD8+, and CD4+FoxP3+ T-cells were assessed prior to dosing and again within 24 hr of the last dose. The CD4+FoxP3+ phenotype was used to identify Treg cells in the periphery, given that FoxP3 expression directly correlates with Treg cell function regardless of CD25 expression levels and because CD25 is also found on activated CD4+ T-cells. In Study B, T-cell subsets were also evaluated at the 12-week end point. We first compared T-cell subset proportions between 2 groups: 1) placebo and 2) all mice that received antibody in Studies B and C. At the time of the last dose, the mice that received anti-CD3 mAb F(ab′)₂ had significantly lower percentages of CD4+ T-cells (placebo: 60.6%±3.3%, treated: 31.6%±2.4%, p<0.001) and CD8+ T-cells (placebo: 19.2%±1.2%, treated: 10.7%±0.6%, p<0.001) in peripheral blood (FIG. 17A). However, there was no significant alteration in the CD4+:CD8+ T-cell ratio when comparing the placebo group to the anti-CD3 mAb F(ab′)-2-treated group as a whole. In contrast, the percentage of CD4+ T-cells in peripheral blood that were FoxP3+ (i.e., Treg cells) was markedly higher in the anti-CD3 mAb F(ab)-2-treated mice (23.0%±1.4%) compared with placebo mice (8.1%±1.0%, p<0.001).

Given the transient decline in total lymphocyte numbers in the peripheral blood and the increased percentage of CD4+FoxP3+ T-cells at the end of dosing, it was hypothesized that CD4+FoxP3+ T-cells were either selectively maintained or expanded as a result of anti-CD3 mAb F(ab′)₂ treatment. At the 12-week end point, flow cytometric analysis of peripheral blood showed that CD4+ and CD8+ T-cell populations had significantly recovered but remained below baseline levels and that the CD4+FoxP3+ T-cell population had diminished (from elevated post-dosing levels) to slightly above baseline levels (Table 7). While significant changes in the proportion of various T-cell subsets in peripheral blood were detected during the dosing period, long-term follow-up of peripheral blood PD parameters did not reveal any long-term changes. Potential differences in T-cell compartments sequestered at the site of inflammation (e.g., the pancreas) were not assessed.

TABLE 7
Assessment of T-cell Subset Populations in Peripheral Blood of Mice Treated with
Anti-CD3 mAb F(ab′)₂ Fragments (5, 25, or 50 μg) in Study B

| Assessment | | CD4+ | CD8+ | CD4+FoxP3+ |
|---|---|---|---|---|
| time | Treatment | (% of lymphocytes) | (% of lymphocytes) | (% of CD4+ cells) |
| Pretreatment | Anti-CD3 mAb F(ab′)₂ | 47.9% (±3.1) | 14.2 (±1.6) | 6.7 (±0.4) |
| | (n = 6) | | | |
| Last dose | 50 μg/24 hr (n = 6) | 15.4% (±2.0) | 9.0 (±1.4) | 40.3 (±5.8) |
| | 25 μg/72 hr (n = 11) | 25.5% (±2.9) | 10.1 (±1.5) | 24.3 (±3.4) |
| | 5 μg/72 hr (n = 10) | 16.8% (±3.1) | 8.9 (±1.4) | 19.3 (±2.0) |
| | Placebo (n = 5) | 54.1 (±3.4) | 19.8 (±0.8) | 6.8 (±0.6) |
| 12-week | 50 μg/24 hr (n = 8) | 32.9 (±2.5) | 11.8 (±2.6) | 9.0 (±2.8) |
| study end | 25 μg/72 hr (n = 6) | 28.7 (±3.1) | 14.8 (±2.1) | 10.0 (±1.7) |
| point | 5 μg/72 hr (n = 8) | 30.2 (±2.7) | 11.8 (±4.3) | 8.9 (±1.6) |

The proportion of CD4+, CD8+, and CD4+FoxP3+ T-cells (mean ± SEM) were measured by flow cytometry in peripheral blood prior to treatment within 24 hr of the last dose and at the 12-week study end point.

The PD parameters observed at completion of dosing were also analyzed by (anti-CD3 mAb F[ab′]₂) dose regimen and according to whether the mice had entered remission or remained diabetic after treatment. Reductions in proportions of CD4+ and CD8+ T-cells and increases in proportions of CD4+FoxP3+ T-cells tended to be greater at higher doses (FIG. 17B). Also, at the higher doses, reductions in CD4+ T-cell proportions were greater than that observed in CD8+ T-cells, resulting in a temporary decrease in the CD4+:CD8+ T-cell ratio. At the 12-week end point, the CD4+:CD8+ T-cell ratio returned to baseline, as both CD4+ and CD8+ T-cell populations had significantly recovered (Table 7). At the lower but still efficacious doses, a decrease in CD4+:CD8+ T-cell ratio was not observed.

Ultimately, unlike the CD3/TCR-complex modulation patterns elicited by varying doses of anti-CD3 mAb F(ab)₂ (FIG. 15B), a strictly dose-dependent relationship for the alterations in proportions of T-cell subsets was not observed. Furthermore, within each dose regimen, proportions of circulating CD4+, CD8+, and CD4+FoxP3+ T-cells at completion of dosing were similar in responder and non-responder mice. However, it is possible that at local sites of inflammation, such as the pancreas and pancreatic lymph nodes, there may be significant differences between responder and non-responder mice in the proportions of these T-cell populations.

Responder Mice have Greater Residual Beta-Cell Function at Initiation of Treatment:

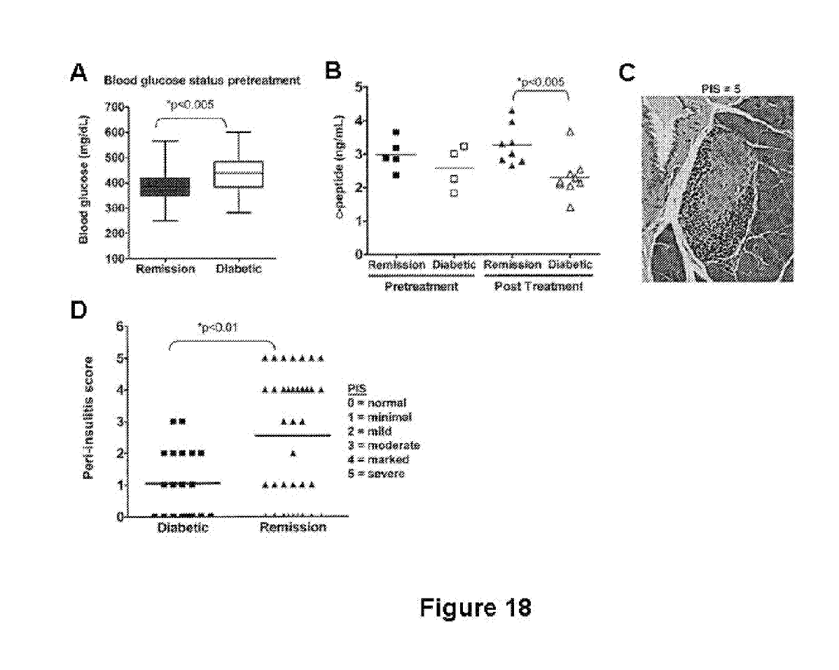

To investigate why some diabetic mice responded to therapy while others did not, even when they experienced similar changes in PD parameters, the pretreatment level of beta-cell function was evaluated by measuring blood glucose and random serum C-peptide levels. As shown in FIG. 18A, pretreatment blood glucose values were significantly lower in mice that entered remission than in those that remained diabetic (mean±standard error of the mean [SEM]: remission 383±9.3 mg/dL, diabetic 441±14.2 mg/dL, p<0.005) (FIG. 18A). This suggests that mice that had a higher level of residual beta-cell function at study entry were more likely to respond to treatment. Similarly, the remission group had higher random serum C-peptide levels than the diabetic group, but this difference was not statistically significant (FIG. 18B). These data suggest that efficacy of treatment may be related to baseline beta-cell function. At the end of the 12-week follow-up period, C-peptide levels were significantly higher in the remission group than in the diabetic group (FIG. 18B).

At the 12-week assessment in Study B, histological sections of pancreas were prepared and evaluated for islet content and the presence of leukocytes within the islets. Eighty-one percent of pancreatic sections from mice that entered remission contained islets (n=43), whereas 74% of pancreatic sections from treated mice that remained diabetic contained islets (n=27). In the placebo group, only 71% of pancreatic sections contained islets (n=14). While these differences were not statistically significant, probably due to the limited number of sections analyzed, the data suggest that the pancreata of non-responders were likely to have fewer preserved islets. Leukocytes present within the islets consisted almost entirely of lymphocytes that were always found at the islet periphery (FIG. 18C), rather than infiltrating throughout the islet as is observed during destructive intra-insulitis. This pattern of peri-insulitis is commonly observed in diabetic mice that have undergone some type of immune therapy. Interestingly, of the anti-CD3 mAb F(ab′)-2-treated mice, those that entered remission had markedly higher scores for peri-insulitis than mice that remained diabetic (FIG. 18D). This suggests that the lymphocytes present in peri-insulitis either are not destructive or are being held at bay by some regulatory mechanism, e.g., by the action of Treg cells.

Discussion:

In this Example, dose-ranging experiments were performed in new-onset diabetic NOD mice to determine if low dose regimens of anti-CD3 mAb F(ab′)₂ were efficacious and to examine potential PD effects associated with remission. Previous studies have shown that a daily dose regimen of 50 μg of anti-CD3 mAb F(ab′)₂ for 5 doses (250 μg total) resulted in high rates of remission. In the dose regimen used in this Example, nearly complete CD3/TCR-complex modulation occurred after the first dose and was sustained throughout the dosing period in peripheral blood. By lowering the dose of anti-CD3 mAb F(ab′)₂ and modifying the dose regimen, a pattern of transient and partial CD3/TCR-complex modulation during dosing that was as efficacious as the higher doses previously established in the literature was achieved. Changes in PD parameters in the peripheral blood of mice treated with anti-CD3 mAb F(ab′)₂, such as a transient decrease in lymphocyte counts, a decrease in the percentage of CD4+ and CD8+ T cells, and a marked increase in the proportion of CD4+FoxP3+ T cells, were present at all dose regimens tested. Furthermore, these PD effects were similar in responder and non-responders, indicating that drug was active in all treated mice. Instead, these data suggest that mice that successfully responded to anti-CD3 mAb F(ab′)₂ treatment had better residual beta-cell function at initiation of treatment. Overall, this Example shows that lower doses of anti-CD3 mAb F(ab′)₂ are as effective in new-onset diabetic NOD mice as the higher doses previously established in the literature.

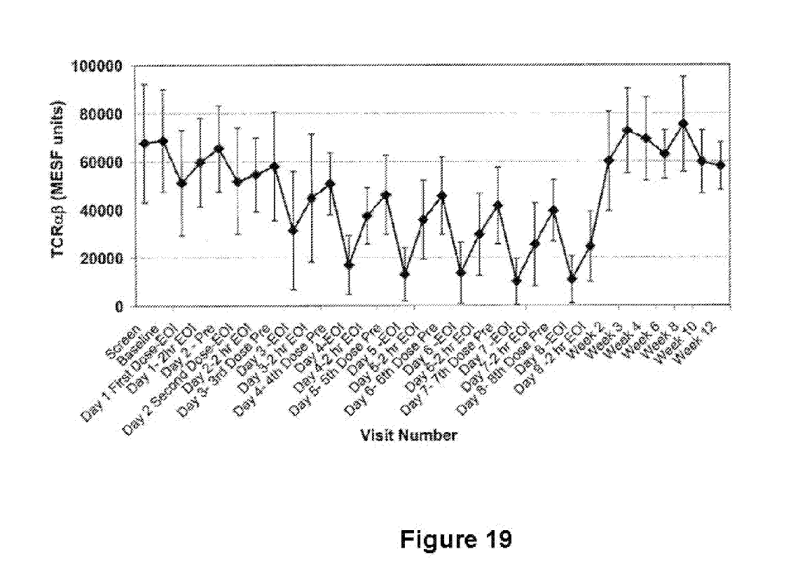

In a Phase 2 clinical study, new-onset type 1 diabetic subjects treated with high doses of otelixizumab had profound and sustained modulation of the CD3/TCR complex throughout the dosing period (Keymeulen, B., Vandemeulebroucke, E., Ziegler, A. G. et al. Insulin needs after CD3-antibody therapy in new-onset type 1 diabetes. N Engl J Med 2005; 352: 2598-608). Otelixizumab-treated subjects had improved beta-cell function as compared with placebo for as long as 18 months after dosing (Id.) and follow-up data showed a significant decrease in exogenous insulin use up to 48 months after dosing (Id.; You, S., Candon, S., Kuhn, C., Bach, J. F. & Chatenoud, L. CD3 antibodies as unique tools to restore self-tolerance in established autoimmunity their mode of action and clinical application in type 1 diabetes. Adv Immunol 2008; 100: 13-37, incorporated herein by reference in its entirety). Modifications of the high dose regimen of otelixizumab used in the BDR study to optimize safety and tolerability have been explored, specifically investigating regimens that result in lower and less sustained levels of CD3/TCR-complex modulation. These optimized otelixizumab dose regimens are associated with a transient pattern of CD3/TCR complex modulation and are very similar to what we describe in this study with the 72-hr dose regimen in mice (FIG. 19B). The safety advantages of lower doses of anti-CD3 mAb are numerous, including greatly reduced cytokine release, the expectation of sustained Epstein-Barr virus (EBV) immunosurveillance, and the lack of immunogenicity which would allow for re-dosing if required. Interestingly, preliminary clinical studies with teplizumab, another Fc-modified anti-CD3 mAb, suggest that higher doses do not improve efficacy and are associated with an increase in adverse events. (See Herold, K. C., Gitelman, S., Greenbaum, C. et al. Treatment of patients with new onset Type 1 diabetes with a single course of anti-CD3 mAb teplizumab preserves insulin production for up to 5 years. Clin Immunol 2009, incorporated herein by reference in its entirety.)

This Example demonstrated that anti-CD3 mAb F(ab′)₂ dose regimens featuring low doses 3 days apart elicited patterns of transient and partial CD3/TCR-complex modulation and resulted in remission rates comparable to the higher doses previously established in the NOD mouse model. Furthermore, even at low doses, remission was durable. A total dose of 8 resulted in 53% long-term remission for up to 24 weeks after treatment. This is comparable to the 56% remission in the 250 μg total dose regimen, despite the >30-fold difference in dose. It has been reported that single high doses (1 dose of 18-50 μg of anti-CD3 mAb F(ab′)₂) produce similarly high remission rates; however, the mice that responded favorably to such treatment were within a very limited glycemia range (300-349 mg/dL) at the start of treatment making a direct comparison with our data difficult.

Various PD parameters were evaluated in mice that received anti-CD3 mAb F(ab′)₂. CD3/TCR-complex modulation on peripheral T-cells was dose-dependent. Interestingly, as little as 30% CD3/TCR-complex modulation, elicited by the 2 μg (4×/72 hr), was sufficient to induce high rates of durable remission in new-onset diabetic NOD mice. The difference in the level of CD3/TCR-complex modulation between the 2 μg (4×/72 hr) dose regimen and the less effective dose regimen of 1 μg (4×/72 hr) was not large, ˜30% vs. 20%, but it was statistically significant. It is estimated that the 2 μg (4×/72 hr) dose regimen results in antibody occupying as little as one-fifth of the total number of CD3 molecules in the mouse. Overall, this work demonstrated that in the NOD mouse model 1) sustained CD3/TCR-complex modulation during the dosing period was not required for efficacy and remission can occur at lower doses that produce only transient CD3/TCR-complex modulation, and 2) partial CD3/TCR-complex modulation on circulating lymphocytes was sufficient to induce remission.

By the end of dosing, there were transient decreases in lymphocyte counts in the peripheral blood, similar to that observed in clinical studies with otelixizumab, but they were not strictly dose dependent. Also, at the end of dosing, there were reductions in the percentages of CD4+ and CD8+ T-cells and a marked increase in the proportion of CD4+FoxP3+ T-cells in the peripheral blood. Similar changes have been observed in new-onset type 1 diabetic subjects administered otelixizumab (Keymeulen, B., Vandemeulebroucke, E., Ziegler, A. G. et al. Insulin needs after CD3-antibody therapy in new-onset type 1 diabetes. N Engl J Med 2005; 352: 2598-608). In NOD mice, the altered proportions of T cell subsets were not strictly dose dependent, although they tended to be more marked at higher doses. Given that similar PD effects occurred in both mice that entered remission and in those that remained diabetic, it is likely that other factors in addition to these PD parameters play a role in predicting response to anti-CD3 mAb F(ab′)₂ treatment in NOD mice. Without wishing to be bound by theory, it is likely that an optimal amount of PD activity (including CD3/TCR-complex modulation, transient loss of circulating lymphocytes, and/or alterations in T-cell subsets) is one factor that determines efficacy, and that efficacy will also be dependent on the level of beta-cell mass and/or function prior to treatment.

That efficacy of anti-CD3 mAb F(ab′)₂ treatment is correlated with residual beta-cell status is supported by the observation that mice with better residual beta-cell function, as measured by blood glucose and serum C-peptide levels, were more likely to respond to treatment. It is also supported by other studies in which NOD mice that remained diabetic after anti-CD3 mAb F(ab′)₂ treatment were restored to full metabolic control with syngeneic islet transplantation. These observations are consistent with findings in the Phase 2 BDR study, where increases in endogenous insulin production were most pronounced in otelixizumab-treated subjects with initial residual beta-cell function at or above the 50th percentile. See Keymeulen, B., Vandemeulebroucke, E., Ziegler, A. G. et al. Insulin needs after CD3-antibody therapy in new-onset type 1 diabetes. N Engl J Med 2005; 352: 2598-608.

Overall, these results demonstrate that low sub-immunogenic doses of anti-CD3 mAb F(ab′)₂ that result in transient and partial CD3/TCR-complex modulation are sufficient to induce high rates of remission in new-onset diabetic NOD mice. While the autoimmune component of type 1 diabetes may be sufficiently resolved with anti-CD3 mAb therapy, glycemic control and functional remission of disease likely depend upon of the level of residual beta-cell function at the time of treatment. Successfully translating anti-CD3 mAb therapy into the clinic may therefore depend not only upon identifying dosing strategies that minimize adverse effects while maximizing efficacy, but also upon identifying the window of treatment during which patients are most likely to respond favorably to treatment.

### Example 8

### Mathematical Definition of Daily Maximal and Minimum Serum Drug (Otelixizumab) Concentrations and Levels of Free and Drug-Bound CD3/TCR Complex on CD4+ and CD8+ T Cells in Patients Undergoing Otelixizumab Treatment for Type I Diabetes

The mathematical definitions described below were based on the pharmacokinetic (PK) data described in Examples 1-7 and obtained from patients undergoing treatment with the otelixizumab anti-CD3 antibody using various dosing and scheduling regimens.

(a) Maximum and Minimum Blood (Serum) Drug (Otelixizumab) Concentrations after Each Daily Dose of Otelixizumab

If one assumes that a drug is administered as a bolus injection to the patient at n different occasions with n different doses (D₁, D₂, . . . , Dₙ) and at the same dosing interval τ and if the drug pharmacokinetics is described by a one compartmental model with a linear elimination, then the concentration after a first dose equals:

$$C ( t ) = \frac{{D}_{1}}{V} exp ( - {k}_{el} t ) \qquad ( 1 )$$

where t denotes time, V is the volume of distribution and kₑₗ is an elimination rate constant. In this case the minimal and maximal concentrations after a first dose are given by:

$${C}_{1 , max} = \frac{{D}_{1}}{V} \\ {C}_{1 , min} = \frac{{D}_{1}}{V} exp ( - {k}_{el} τ ) \qquad ( 2 )$$

The administration of a second dose at time t=τ leads to the new maximum being a sum of the concentration at the time of administration (C_{1,min}) and the concentration increase due to the next dose given (D₂/V):

$${C}_{2 , max} = {C}_{1 , min} + \frac{{D}_{2}}{V} = \frac{{D}_{1}}{V} exp ( - {k}_{el} τ ) + \frac{{D}_{2}}{V} \\ {C}_{2 , min} = {C}_{2 , min} exp ( - {k}_{el} τ ) = \frac{{D}_{1}}{V} exp ( - 2 {k}_{el} τ ) + \frac{{D}_{2}}{V} exp ( - {k}_{el} τ ) \qquad ( 3 )$$

Similarly for second dose administered at t=2τ leads to:

$${C}_{3 , max} = {C}_{2 , min} + \frac{{D}_{3}}{V} = \frac{{D}_{1}}{V} exp ( - 2 {k}_{el} τ ) + \frac{{D}_{2}}{V} exp ( - {k}_{el} τ ) + \frac{{D}_{3}}{V} \\ {C}_{3 , min} = {C}_{3 , min} exp ( - {k}_{el} τ ) = \frac{{D}_{1}}{V} exp ( - 3 {k}_{el} τ ) + \frac{{D}_{2}}{V} exp ( - 2 {k}_{el} τ ) + \frac{{D}_{3}}{V} exp ( - {k}_{el} τ ) \qquad ( 4 )$$

After n^{th} administration (at time t=(n−1)τ) we have

$${C}_{n , max} = \frac{{D}_{1}}{V} exp ( - ( n - 1 ) {k}_{el} τ ) + \frac{{D}_{2}}{V} exp ( - ( n - 2 ) {k}_{el} τ ) + … + \frac{{D}_{n}}{V} \\ {C}_{n , min} = \frac{{D}_{1}}{V} exp ( - {nk}_{el} τ ) + \frac{{D}_{2}}{V} exp ( - ( n - 1 ) {k}_{el} τ ) + … + \frac{{D}_{n}}{V} exp ( - {k}_{el} τ ) \qquad ( 5 )$$

It can be written in a compact way as:

$${C}_{n , max} = {\sum }_{i = 1}^{n} \frac{{D}_{i}}{V} exp ( - ( n - i ) {k}_{el} τ ) \\ {C}_{n , min} = {\sum }_{i = 1}^{n} \frac{{D}_{i}}{V} exp ( - ( n - i + 1 ) {k}_{el} τ ) \qquad ( 6 )$$

To use equation (6), the volume of distribution and elimination rate constant (or half-live) need to be known. When a concentration after the first dose is known, i.e. it equals C₀=D₁/V, then the Cₘₐₓ and Cₘᵢₙ at the n^{th} administration can be reparameterized to:

$${C}_{n , max} = {C}_{0} \cdot {\sum }_{i = 1}^{n} \frac{{D}_{i}}{{D}_{1}} exp ( - ( n - i ) {k}_{el} τ ) \\ {C}_{n , min} = {C}_{0} \cdot {\sum }_{i = 1}^{n} \frac{{D}_{i}}{{D}_{1}} exp ( - ( n - i + 1 ) {k}_{el} τ ) \qquad ( 7 )$$

In this case, only the elimination rate constant (or half-life) needs to be known. For otelixizumab, the half-life in the low dosing equals 0.50 day and volume of distribution 13.9 L. The maximal and minimal concentrations for a typical subject were calculated using equation (6) and these values of half life and volume of distribution (all the studies listed in Table 2, except the BDR study). For graphs of BDR studies (BDR Group A and BDR Group B), a longer half-life (1.52 day) and a volume of distribution of 7.56 L were used.

In this way, Cmin and Cmax after every dose in the following studies were calculated: Cohort C (also referred to as Cohort RT-C), Cohort CH1 (also referred to as TTEDD CH1), Cohort CH2 (also referred to as TTEDD CH2), Cohort CH3 (also referred to as TTEDD CH3), Cohort CH4 (also referred to as TTEDD CH4), and Cohort CH5 (also referred to as TTEDD CH5). In addition, the values for the BDR, Group A (also referred to as Study 1, Group A; six daily doses of 24 mg, 8 mg, 8 mg, 8 mg, 8 mg, and 8 mg) and BDR, Group B (also referred to as Study 1, Group B; six daily doses of 8 mg each) were determined.

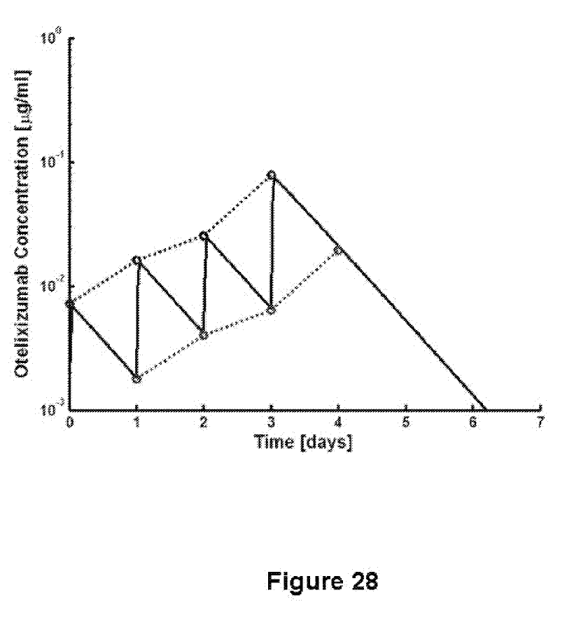

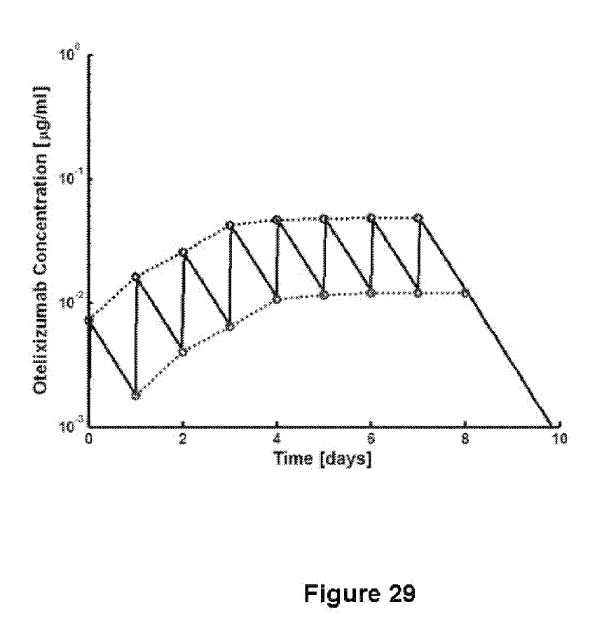

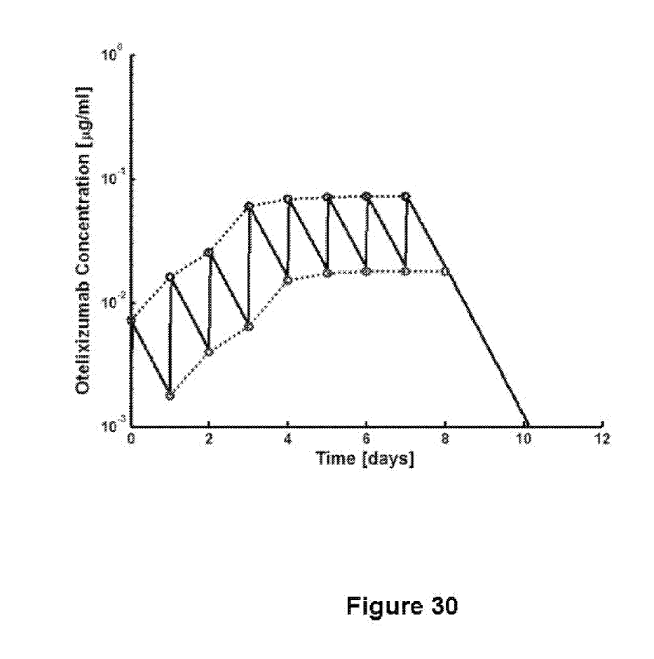

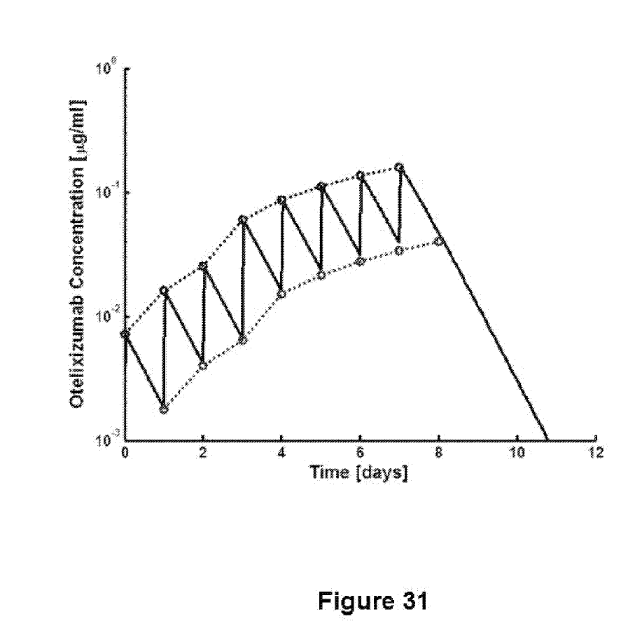

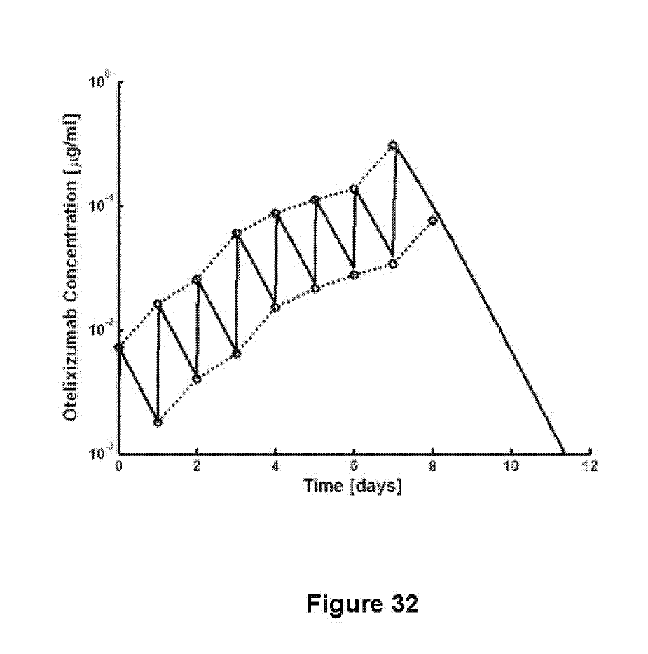

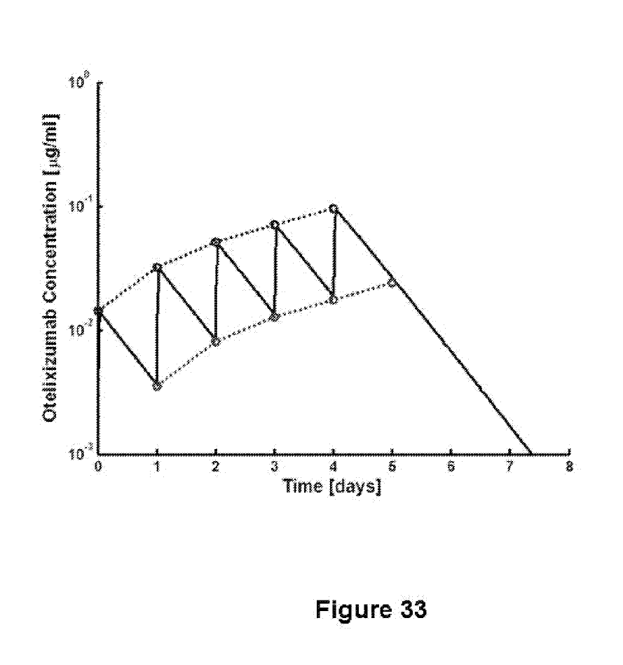

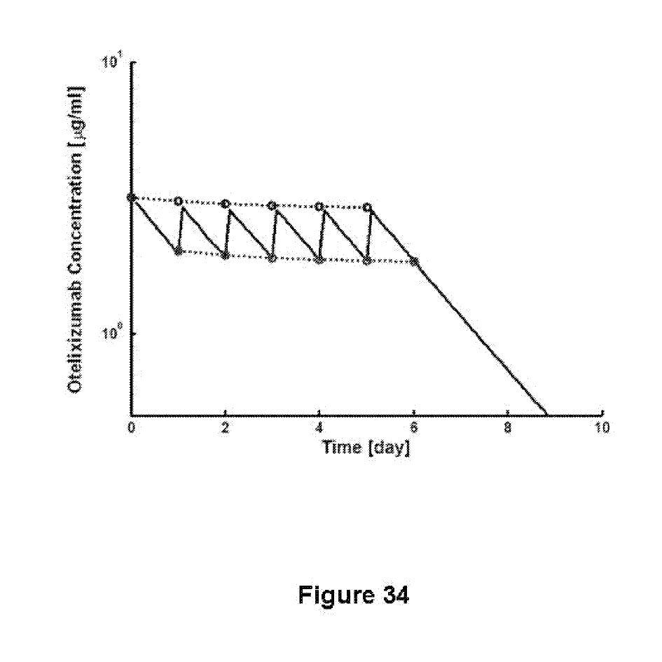

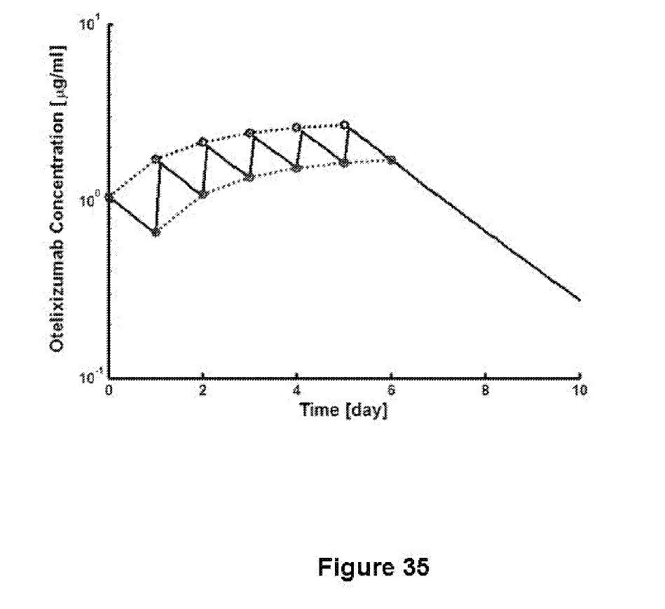

The calculated data are shown below in Tables 8 to 15 and in graphical form in FIGS. 28 to 35.

TABLE 8
Cₘᵢₙ and Cₘₐₓ for each daily dose of otelixizumab in Cohort C (RT-C)

| Nr. | Dose [mg] | Cₘᵢₙ [mg/L] | Cₘₐₓ [mg/L] |
|---|---|---|---|
| 1 | 0.1 | 0.002 | 0.007 |
| 2 | 0.2 | 0.004 | 0.016 |
| 3 | 0.3 | 0.006 | 0.026 |
| 4 | 1 | 0.02 | 0.078 |

TABLE 9
Cₘᵢₙ and Cₘₐₓ for each daily dose of otelixizumab in TTEDD CH1

| Nr. | Dose [mg] | Cₘᵢₙ [mg/L] | Cₘₐₓ [mg/L] |
|---|---|---|---|
| 1 | 0.1 | 0.002 | 0.007 |
| 2 | 0.2 | 0.004 | 0.016 |
| 3 | 0.3 | 0.006 | 0.026 |
| 4 | 0.5 | 0.011 | 0.042 |
| 5 | 0.5 | 0.012 | 0.047 |
| 6 | 0.5 | 0.012 | 0.048 |
| 7 | 0.5 | 0.012 | 0.048 |
| 8 | 0.5 | 0.012 | 0.048 |

TABLE 10
Cₘᵢₙ and Cₘₐₓ for each daily dose of otelixizumab in TTEDD CH2

| Nr. | Dose [mg] | Cₘᵢₙ [mg/L] | Cₘₐₓ [mg/L] |
|---|---|---|---|
| 1 | 0.1 | 0.002 | 0.007 |
| 2 | 0.2 | 0.004 | 0.016 |
| 3 | 0.3 | 0.006 | 0.026 |
| 4 | 0.75 | 0.015 | 0.060 |
| 5 | 0.75 | 0.017 | 0.069 |
| 6 | 0.75 | 0.018 | 0.071 |
| 7 | 0.75 | 0.018 | 0.072 |
| 8 | 0.75 | 0.018 | 0.072 |

TABLE 11
Cₘᵢₙ and Cₘₐₓ for each daily dose of otelixizumab in TTEDD CH3

| Nr. | Dose [mg] | Cₘᵢₙ [mg/L] | Cₘₐₓ [mg/L] |
|---|---|---|---|
| 1 | 0.1 | 0.002 | 0.007 |
| 2 | 0.2 | 0.004 | 0.016 |
| 3 | 0.3 | 0.006 | 0.026 |
| 4 | 0.75 | 0.015 | 0.06 |
| 5 | 1 | 0.022 | 0.087 |
| 6 | 1.25 | 0.028 | 0.112 |
| 7 | 1.5 | 0.034 | 0.136 |
| 8 | 1.75 | 0.040 | 0.16 |

TABLE 12
Cₘᵢₙ and Cₘₐₓ for each daily dose of otelixizumab in TTEDD CH4

| Nr. | Dose [mg] | Cₘᵢₙ [mg/L] | Cₘₐₓ [mg/L] |
|---|---|---|---|
| 1 | 0.1 | 0.002 | 0.007 |
| 2 | 0.2 | 0.004 | 0.016 |
| 3 | 0.3 | 0.006 | 0.026 |
| 4 | 0.75 | 0.015 | 0.060 |
| 5 | 1 | 0.022 | 0.087 |
| 6 | 1.25 | 0.028 | 0.112 |
| 7 | 1.5 | 0.034 | 0.136 |
| 8 | 3.75 | 0.076 | 0.304 |

TABLE 13
Cₘᵢₙ and Cₘₐₓ for each daily dose of otelixizumab in TTEDD CH5

| Nr. | Dose [mg] | Cₘᵢₙ [mg/L] | Cₘₐₓ [mg/L] |
|---|---|---|---|
| 1 | 0.2 | 0.004 | 0.014 |
| 2 | 0.4 | 0.008 | 0.032 |
| 3 | 0.6 | 0.013 | 0.051 |
| 4 | 0.8 | 0.018 | 0.070 |
| 5 | 1.1 | 0.024 | 0.097 |

TABLE 14
Cₘᵢₙ and Cₘₐₓ for each daily dose of otelixizumab in BDR Group A

| Nr. | Dose [mg] | Cₘᵢₙ [mg/L] | Cₘₐₓ [mg/L] |
|---|---|---|---|
| 1 | 24 | 2.010 | 3.175 |
| 2 | 8 | 1.943 | 3.068 |
| 3 | 8 | 1.900 | 3.001 |
| 4 | 8 | 1.873 | 2.958 |
| 5 | 8 | 1.856 | 2.931 |
| 6 | 8 | 1.845 | 2.914 |

TABLE 15
Cₘᵢₙ and Cₘₐₓ for each daily dose of otelixizumab in BDR Group B

| Nr. | Dose [mg] | Cₘᵢₙ [mg/L] | Cₘₐₓ [mg/L] |
|---|---|---|---|
| 1 | 8 | 0.670 | 1.058 |
| 2 | 8 | 1.094 | 1.728 |
| 3 | 8 | 1.363 | 2.152 |
| 4 | 8 | 1.533 | 2.421 |
| 5 | 8 | 1.641 | 2.591 |
| 6 | 8 | 1.709 | 2.699 |

(b) Maximum and Minimum Levels of Free and Drug-Bound CD3/TCR Complexes on CD4+ and CD8 T Cells after Each Daily Dose of Otelixizumab

Using the minimal and maximal concentrations calculated based on equation 6 above, the PK/PD model proposed for otelixizumab suggests that, for maximal concentrations, the minimum value of free receptors and the maximal value of drug receptor complexes are observed. Similarly for minimal concentrations the maximal value of free receptors and the minimal value of drug receptor complexes are observed. This assumption leads to the following equations describing the maximum/minimum values for receptor dynamics (these equations are based on equations 10 and 11 in Wiczling et al. (2010) J. Clin. Pharmacol. 50, 494, the disclosure of which is incorporated herein by reference in its entirety).

$$\% {FR}_{n . min} = 100 ( 1 - \frac{{C}_{n , max}}{{IC}_{50 , FR} + {C}_{n , max}} )$$

$$\% {FR}_{n , max} = 100 ( 1 - \frac{{C}_{n , min}}{{IC}_{50 , FR} + {C}_{n , min}} )$$

$${DR}_{n , max} = {SCL}_{1} \cdot {MFR}_{0} ( 1 - \frac{{C}_{n , max}}{{IC}_{50 , FR} + {C}_{n , max}} ) \cdot {C}_{n , max}$$

$${DR}_{n , min} = {SCL}_{1} \cdot {MFR}_{0} ( 1 - \frac{{C}_{n , min}}{{IC}_{50 , FR} + {C}_{n , min}} ) \cdot {C}_{n , min}$$

The parameters MFR₀, IC_{50,FR} and SCL1 are taken from Table II of Wiczling et al., supra)

| Parameter [units] | Population typical value |
|---|---|
| IC_{50,FR,CD4} [μg/mL] | 0.0144 |
| IC_{50,FR,CD8} [μg/mL] | 0.0162 |
| MFR_{0,CD4}[10⁵ MESF] | 3.39 |
| MFR_{0,CD8} [10⁵ MESF] | 2.42 |
| SCL_{1,} [(μg/mL)⁻¹] | 105 |

In this way, the FR4max (the maximum level of free receptors on CD4+ T cells), the FR4 min (the minimum level of free receptors on CD4+ T cells), the FR8max (the maximum level of free receptors on CD8+ T cells), the FR8 min (the minimum level of free receptors on CD8+ T cells), the DR4 min (the minimum level of drug bound receptors on CD4+ T cells), DR4max (the maximum level of drug bound receptors on CD4+ T cells), DR8 min (the minimum level of drug bound receptors on CD8+ T cells) and DR8max (the maximum level of drug bound receptors on CD8+ T cells) after every dose in the following studies were calculated: Cohort C (also referred to as Cohort RT-C), Cohort CH1 (also referred to as TTEDD CH1), Cohort CH2 (also referred to as TTEDD), Cohort CH3 (also referred to as TTEDD CH3), Cohort CH4 (also referred to as TTEDD CH4), and Cohort CH5 (also referred to as TTEDD CH5). In addition, the values for the BDR, Group A (also referred to as Study 1, Group A; six daily doses of 24 mg, 8 mg, 8 mg, 8 mg, 8 mg, and 8 mg) and BDR, Group B (also referred to as Study 1, Group B; six daily doses of 8 mg each) were determined.

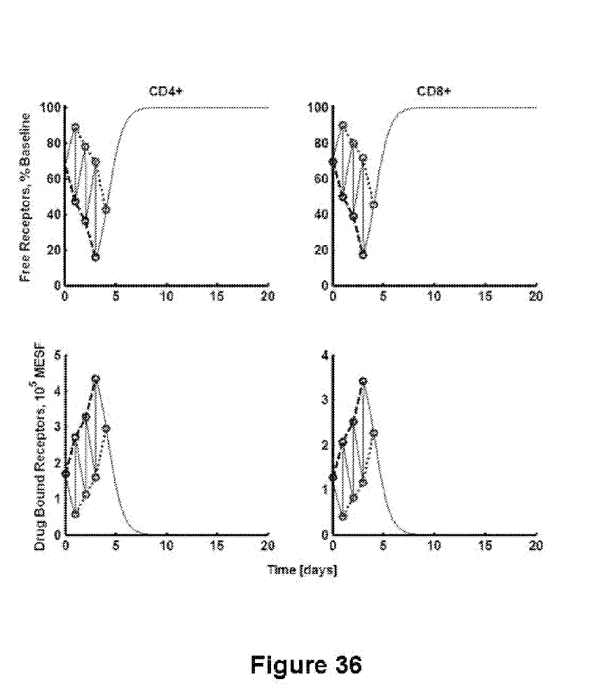

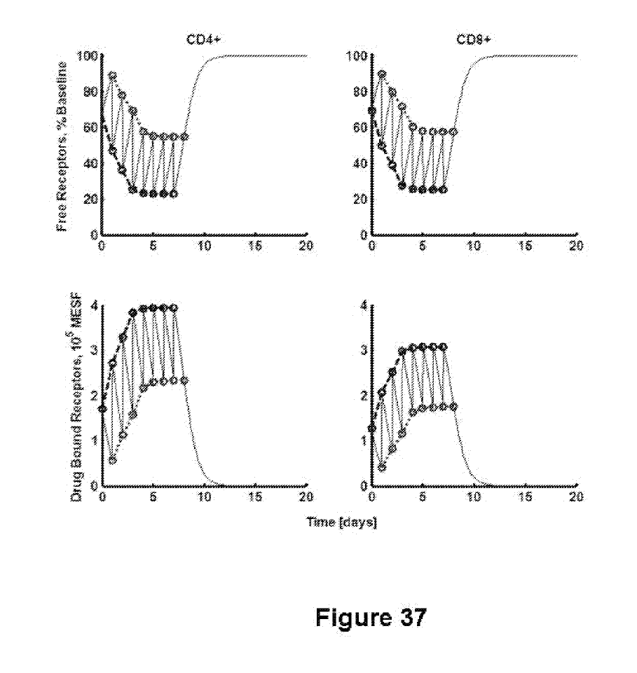

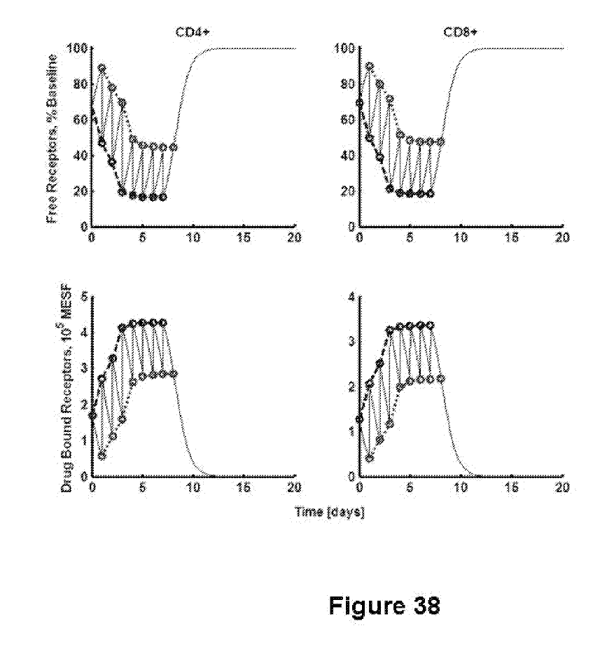

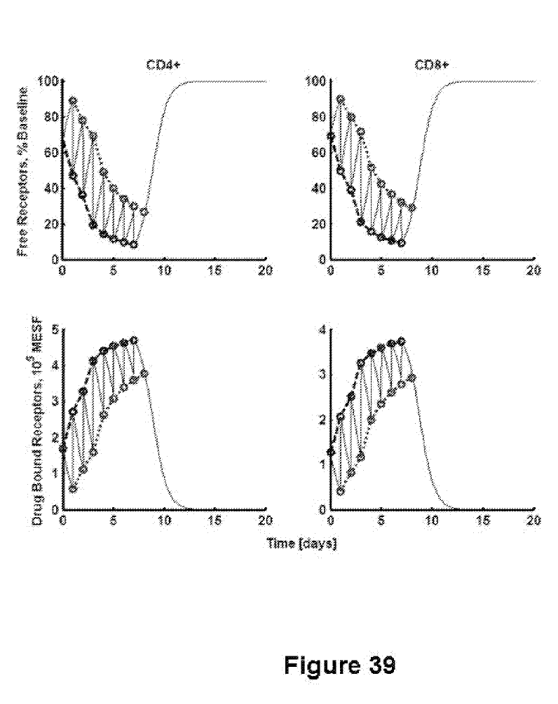

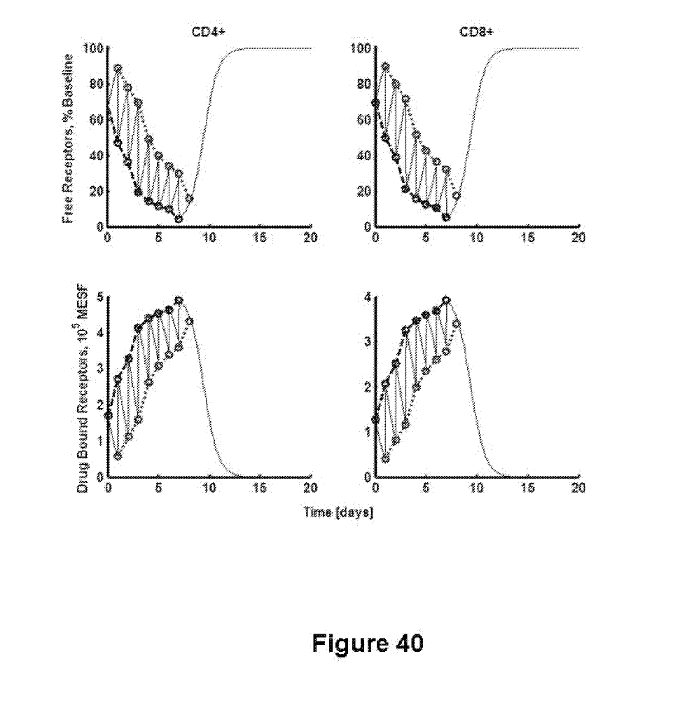

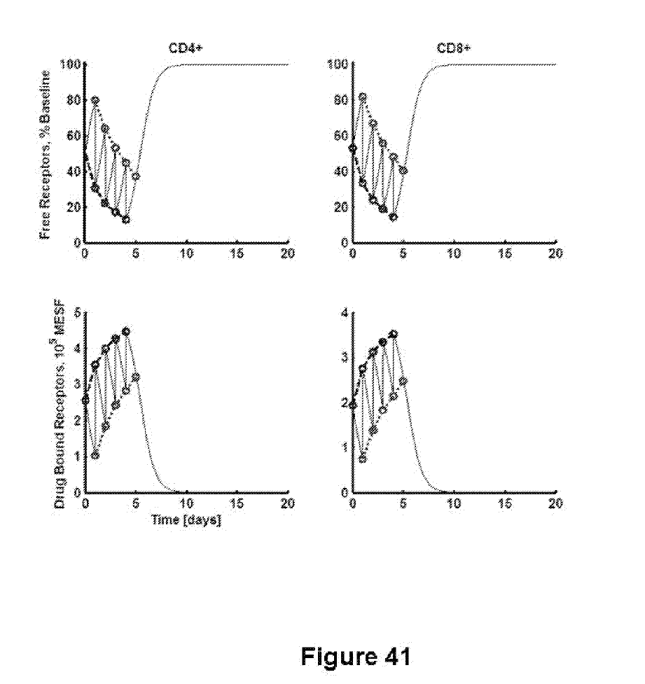

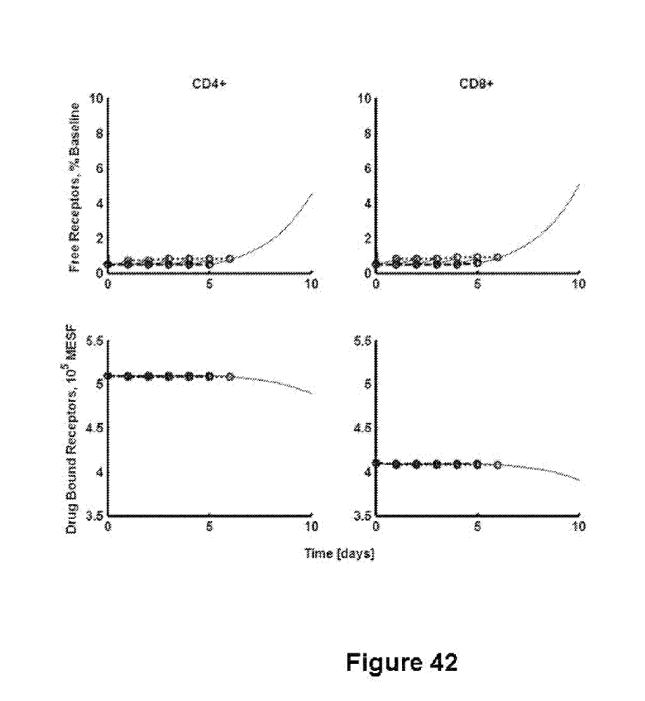

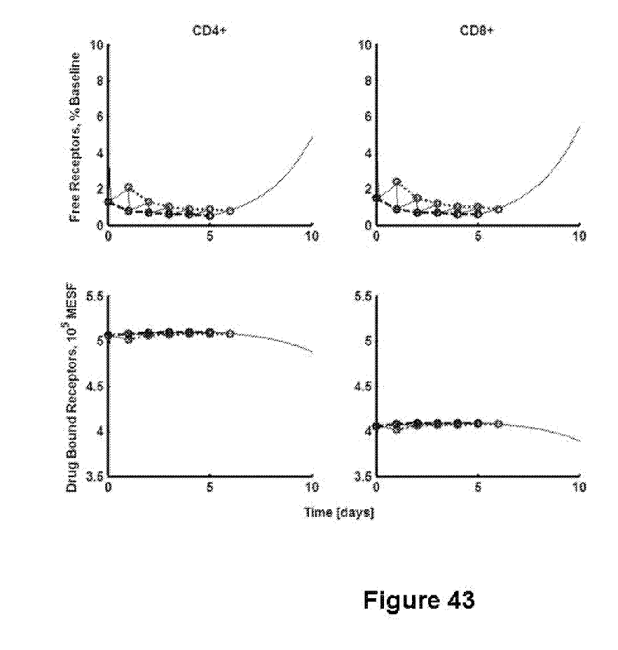

The calculated data are shown below in Tables 16 to 23, below, and in graphical form in FIGS. 36 to 43.

TABLE 16
Maximum and Minimum Levels of Free Receptors (FR) and Drug-Bound Receptors (DR) on
CD4+ and CD8+ T cells for Each Daily Dose of Otelixizumab in Cohort C

| | | | | | | DR4min | DR4max | DR8min | DR8max |
|---|---|---|---|---|---|---|---|---|---|
| | Dose | FR4max | FR4min | FR8max | FR8min | [105 | [105 | [105 | [105 |
| No. | [mg] | [%] | [%] | [%] | [%] | MESF] | MESF] | MESF] | MESF] |
| 1 | 0.1 | 88.9 | 66.7 | 90.0 | 69.2 | 0.569 | 1.71 | 0.411 | 1.27 |
| 2 | 0.2 | 78.0 | 47.1 | 80.0 | 50.0 | 1.12 | 2.71 | 0.823 | 2.06 |
| 3 | 0.3 | 69.2 | 36.0 | 71.6 | 38.7 | 1.58 | 3.28 | 1.17 | 2.52 |
| 4 | 1 | 42.3 | 15.5 | 45.2 | 17.1 | 2.95 | 4.33 | 2.25 | 3.41 |

TABLE 17
Maximum and Minimum Levels of Free Receptors (FR) and Drug-Bound
Receptors (DR) on CD4+ and CD8+ T cells for Each Daily Dose of
Otelixizumab in TTEDD CH1

| | | | | | | DR4ₘᵢₙ | DR4ₘₐₓ | DR8ₘᵢₙ | DR8ₘₐₓ |
|---|---|---|---|---|---|---|---|---|---|
| | Dose | FR4ₘₐₓ | FR4ₘᵢₙ | FR8ₘₐₓ | FR8ₘᵢₙ | [10⁵ | [10⁵ | [10⁵ | [10⁵ |
| No. | [mg] | [%] | [%] | [%] | [%] | MESF] | MESF] | MESF] | MESF] |
| 0 | 0.1 | 88.9 | 66.7 | 90.0 | 69.2 | 0.57 | 1.71 | 0.41 | 1.27 |
| 1 | 0.2 | 78.1 | 47.1 | 80.0 | 50.0 | 1.12 | 2.71 | 0.82 | 2.06 |
| 2 | 0.3 | 69.2 | 36.0 | 71.7 | 38.7 | 1.58 | 3.28 | 1.17 | 2.52 |
| 3 | 0.5 | 57.6 | 25.4 | 60.5 | 27.7 | 2.17 | 3.83 | 1.63 | 2.98 |
| 4 | 0.5 | 55.3 | 23.6 | 58.2 | 25.8 | 2.29 | 3.92 | 1.72 | 3.05 |
| 5 | 0.5 | 54.7 | 23.2 | 57.6 | 25.4 | 2.32 | 3.94 | 1.74 | 3.07 |
| 6 | 0.5 | 54.6 | 23.1 | 57.5 | 25.3 | 2.33 | 3.94 | 1.75 | 3.08 |
| 7 | 0.5 | 54.6 | 23.1 | 57.5 | 25.3 | 2.33 | 3.94 | 1.75 | 3.08 |

TABLE 18
Maximum and Minimum Levels of Free Receptors (FR) and Drug-Bound Receptors (DR) on
CD4+ and CD8+ T cells for Each Daily Dose of Otelixizumab in TTEDD CH2

| | | | | | | DR4min | DR4max | DR8min | DR8max |
|---|---|---|---|---|---|---|---|---|---|
| | Dose | FR4max | FR4min | FR8max | FR8min | [10⁵ | [10⁵ | [10⁵ | [10⁵ |
| No. | [mg] | [%] | [%] | [%] | [%] | MESF] | MESF] | MESF] | MESF] |
| 1 | 0.1 | 88.9 | 66.7 | 90.0 | 69.2 | 0.57 | 1.71 | 0.41 | 1.27 |
| 2 | 0.2 | 78.1 | 47.1 | 80.0 | 50.0 | 1.12 | 2.71 | 0.82 | 2.06 |
| 3 | 0.3 | 69.2 | 36.0 | 71.7 | 38.7 | 1.58 | 3.28 | 1.17 | 2.52 |
| 4 | 0.75 | 48.8 | 19.3 | 51.8 | 21.2 | 2.62 | 4.14 | 1.99 | 3.25 |
| 5 | 0.75 | 45.5 | 17.3 | 48.4 | 19.0 | 2.79 | 4.24 | 2.12 | 3.33 |
| 6 | 0.75 | 44.7 | 16.8 | 47.6 | 18.5 | 2.83 | 4.26 | 2.16 | 3.35 |
| 7 | 0.75 | 44.5 | 16.7 | 47.4 | 18.4 | 2.84 | 4.27 | 2.16 | 3.36 |
| 8 | 0.75 | 44.5 | 16.7 | 47.4 | 18.4 | 2.85 | 4.27 | 2.17 | 3.36 |

TABLE 19
Maximum and Minimum Levels of Free Receptors (FR) and Drug-Bound Receptors (DR) on
CD4+ and CD8+ T cells for Each Daily Dose of Otelixizumab in TTEDD CH3

| | | | | | | DR4min | DR4max | DR8min | DR8max |
|---|---|---|---|---|---|---|---|---|---|
| | Dose | FR4max | FR4min | FR8max | FR8min | [10⁵ | [10⁵ | [10⁵ | [10⁵ |
| No. | [mg] | [%] | [%] | [%] | [%] | MESF] | MESF] | MESF] | MESF] |
| 1 | 0.1 | 88.9 | 66.7 | 90.0 | 69.2 | 0.57 | 1.71 | 0.41 | 1.27 |
| 2 | 0.2 | 78.1 | 47.1 | 80.0 | 50.0 | 1.12 | 2.71 | 0.82 | 2.06 |
| 3 | 0.3 | 69.2 | 36.0 | 71.7 | 38.7 | 1.58 | 3.28 | 1.17 | 2.52 |
| 4 | 0.75 | 48.8 | 19.3 | 51.8 | 21.2 | 2.62 | 4.14 | 1.99 | 3.25 |
| 5 | 1 | 39.8 | 14.2 | 42.7 | 15.7 | 3.08 | 4.40 | 2.36 | 3.47 |
| 6 | 1.25 | 34.0 | 11.4 | 36.7 | 12.7 | 3.38 | 4.54 | 2.61 | 3.60 |
| 7 | 1.5 | 29.8 | 9.6 | 32.3 | 10.7 | 3.60 | 4.63 | 2.79 | 3.68 |
| 8 | 1.75 | 26.5 | 8.3 | 28.8 | 9.2 | 3.77 | 4.70 | 2.93 | 3.74 |

TABLE 20
Maximum and Minimum Levels of Free Receptors (FR) and Drug-Bound Receptors (DR) on
CD4+ and CD8+ T cells for Each Daily Dose of Otelixizumab in TTEDD CH4

| | | | | | | DR4min | DR4max | DR8min | DR8max |
|---|---|---|---|---|---|---|---|---|---|
| | Dose | FR4max | FR4min | FR8max | FR8min | [10⁵ | [10⁵ | [10⁵ | [10⁵ |
| No. | [mg] | [%] | [%] | [%] | [%] | MESF] | MESF] | MESF] | MESF] |
| 1 | 0.1 | 88.9 | 66.7 | 90.0 | 69.2 | 0.57 | 1.71 | 0.41 | 1.27 |
| 2 | 0.2 | 78.1 | 47.1 | 80.0 | 50.0 | 1.12 | 2.71 | 0.82 | 2.06 |
| 3 | 0.3 | 69.2 | 36.0 | 71.7 | 38.7 | 1.58 | 3.28 | 1.17 | 2.52 |
| 4 | 0.75 | 48.8 | 19.3 | 51.8 | 21.2 | 2.62 | 4.14 | 1.99 | 3.25 |
| 5 | 1 | 39.8 | 14.2 | 42.7 | 15.7 | 3.08 | 4.40 | 2.36 | 3.47 |
| 6 | 1.25 | 34.0 | 11.4 | 36.7 | 12.7 | 3.38 | 4.54 | 2.61 | 3.60 |
| 7 | 1.5 | 29.8 | 9.6 | 32.3 | 10.7 | 3.60 | 4.63 | 2.79 | 3.68 |
| 8 | 3.75 | 15.9 | 4.5 | 17.6 | 5.1 | 4.31 | 4.89 | 3.39 | 3.91 |

TABLE 21
Maximum and Minimum Levels of Free Receptors (FR) and Drug-Bound Receptors (DR) on
CD4+ and CD8+ T cells for Each Daily Dose of Otelixizumab in TTEDD CH5

| | | | | | | DR4min | DR4max | DR8min | DR8max |
|---|---|---|---|---|---|---|---|---|---|
| | Dose | FR4max | FR4min | FR8max | FR8min | [10⁵ | [10⁵ | [10⁵ | [10⁵ |
| No. | [mg] | [%] | [%] | [%] | [%] | MESF] | MESF] | MESF] | MESF] |
| 1 | 0.2 | 80.0 | 50.0 | 81.8 | 53.0 | 1.02 | 2.56 | 0.75 | 1.94 |
| 2 | 0.4 | 64.0 | 30.8 | 66.7 | 33.3 | 1.84 | 3.55 | 1.37 | 2.74 |
| 3 | 0.6 | 52.9 | 21.9 | 55.8 | 24.0 | 2.41 | 4.00 | 1.82 | 3.13 |
| 4 | 0.8 | 45.0 | 17.0 | 47.9 | 18.7 | 2.82 | 4.26 | 2.14 | 3.35 |
| 5 | 1.1 | 37.3 | 13.0 | 40.1 | 14.3 | 3.21 | 4.46 | 2.47 | 3.53 |

TABLE 22
Maximum and Minimum Levels of Free Receptors (FR) and Drug-Bound Receptors (DR) on
CD4+ and CD8+ T cells for Each Daily Dose of Otelixizumab in BDR, Group A

| | | | | | | DR4min | DR4max | DR8min | DR8max |
|---|---|---|---|---|---|---|---|---|---|
| | Dose | FR4max | FR4min | FR8max | FR8min | [10⁵ | [10⁵ | [10⁵ | [10⁵ |
| No. | [mg] | [%] | [%] | [%] | [%] | MESF] | MESF] | MESF] | MESF] |
| 1 | 24 | 0.71 | 0.45 | 0.80 | 0.51 | 5.09 | 5.10 | 4.08 | 4.10 |
| 2 | 8 | 0.73 | 0.47 | 0.83 | 0.52 | 5.09 | 5.10 | 4.08 | 4.09 |
| 3 | 8 | 0.75 | 0.48 | 0.84 | 0.54 | 5.09 | 5.10 | 4.08 | 4.09 |
| 4 | 8 | 0.76 | 0.48 | 0.86 | 0.54 | 5.09 | 5.10 | 4.08 | 4.09 |
| 5 | 8 | 0.77 | 0.49 | 0.86 | 0.55 | 5.09 | 5.10 | 4.08 | 4.09 |
| 6 | 8 | 0.77 | 0.49 | 0.87 | 0.55 | 5.09 | 5.10 | 4.08 | 4.09 |

TABLE 23
Maximum and Minimum Levels of Free Receptors (FR) and Drug-Bound Receptors (DR) on
CD4+ and CD8+ T cells for Each Daily Dose of Otelixizumab in BDR, Group B

| | | | | | | DR4min | DR4max | DR8min | DR8max |
|---|---|---|---|---|---|---|---|---|---|
| | Dose | FR4max | FR4min | FR8max | FR8min | [10⁵ | [10⁵ | [10⁵ | [10⁵ |
| No. | [mg] | [%] | [%] | [%] | [%] | MESF] | MESF] | MESF] | MESF] |
| 1 | 8 | 2.10 | 1.34 | 2.36 | 1.51 | 5.02 | 5.06 | 4.02 | 4.05 |
| 2 | 8 | 1.30 | 0.83 | 1.46 | 0.93 | 5.06 | 5.08 | 4.06 | 4.08 |
| 3 | 8 | 1.04 | 0.66 | 1.17 | 0.75 | 5.07 | 5.09 | 4.07 | 4.09 |
| 4 | 8 | 0.93 | 0.59 | 1.04 | 0.66 | 5.08 | 5.10 | 4.07 | 4.09 |
| 5 | 8 | 0.87 | 0.55 | 0.98 | 0.62 | 5.08 | 5.10 | 4.08 | 4.09 |
| 6 | 8 | 0.83 | 0.53 | 0.94 | 0.60 | 5.08 | 5.10 | 4.08 | 4.09 |

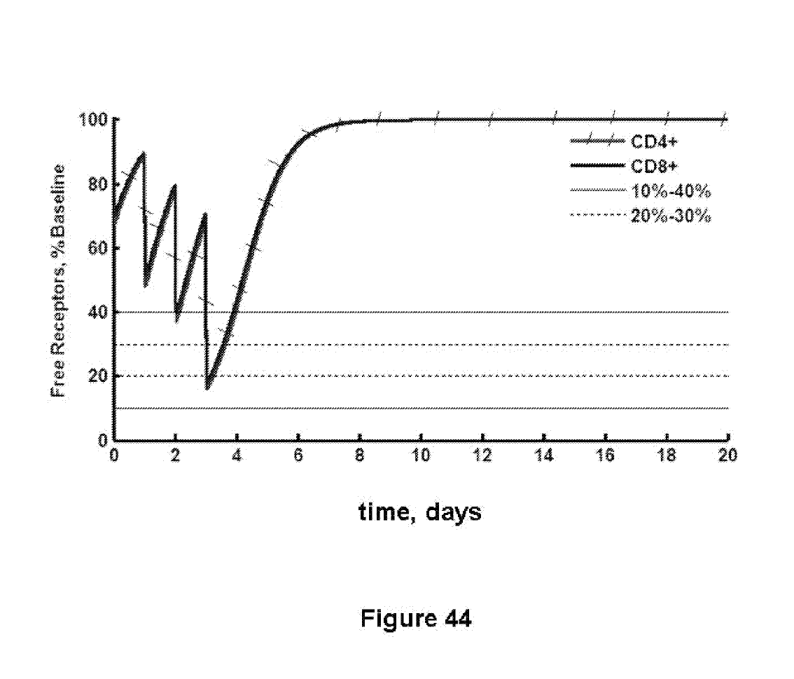

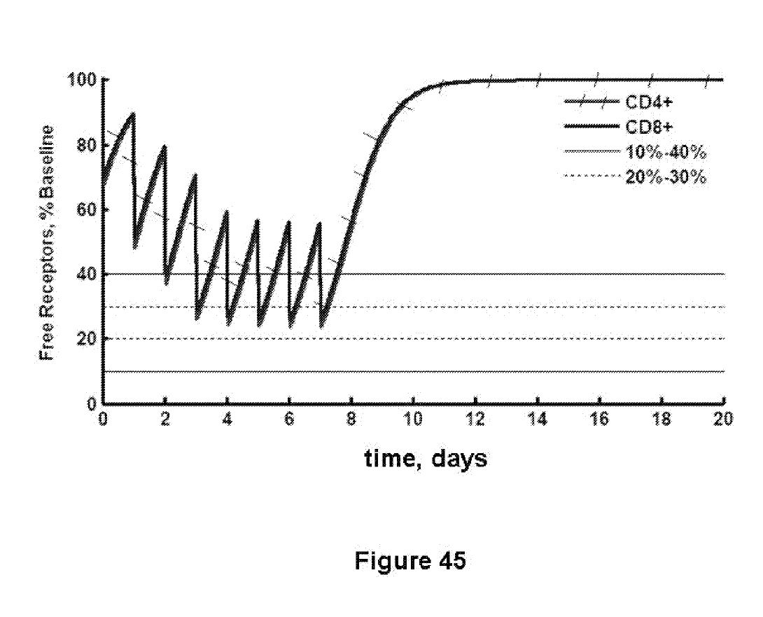

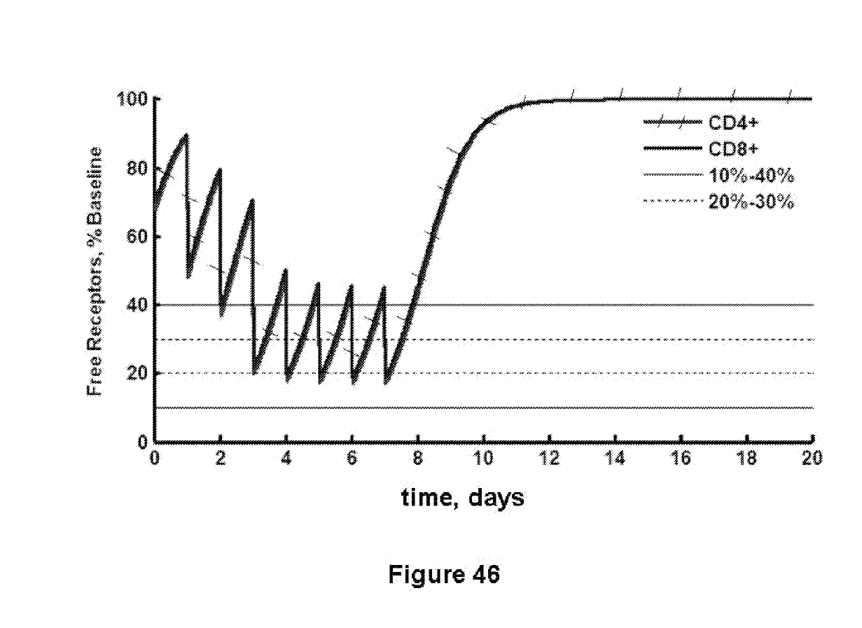

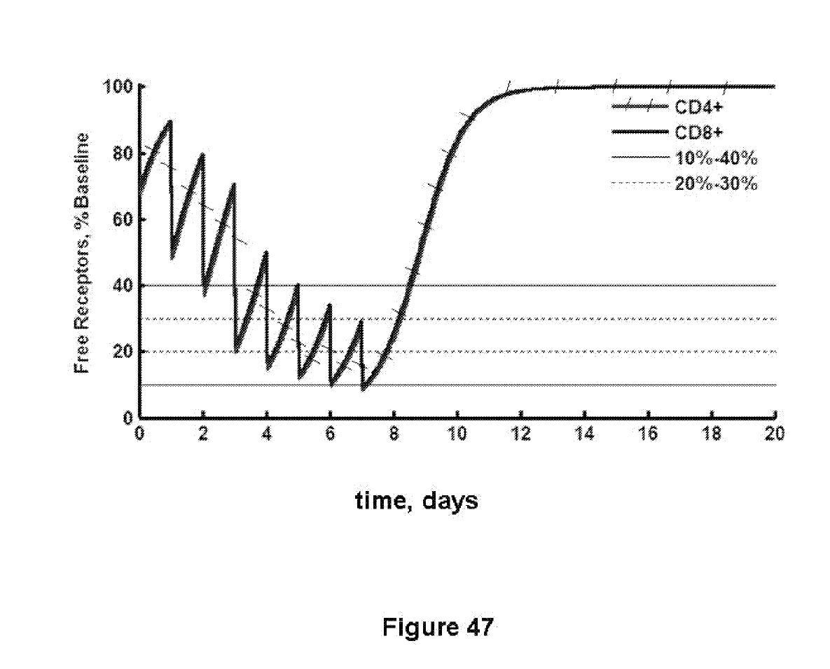

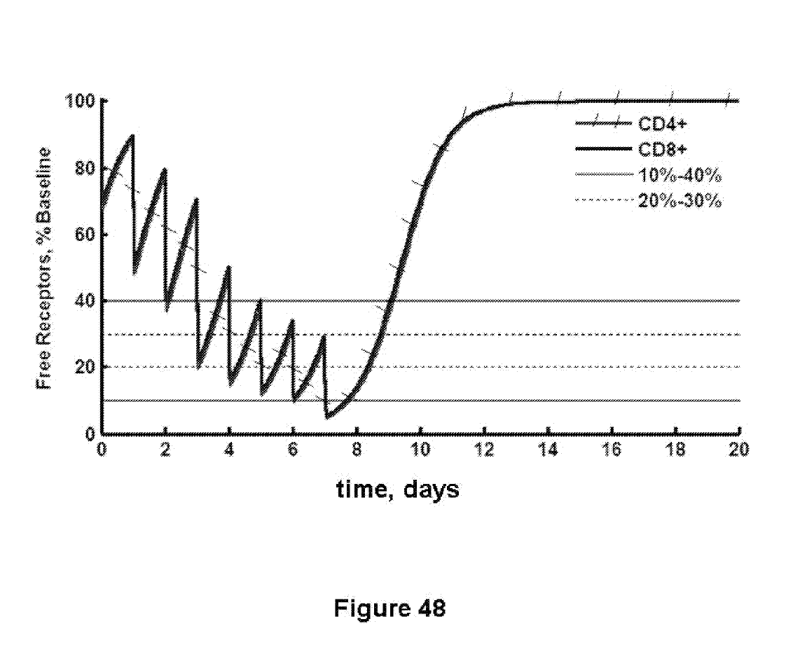

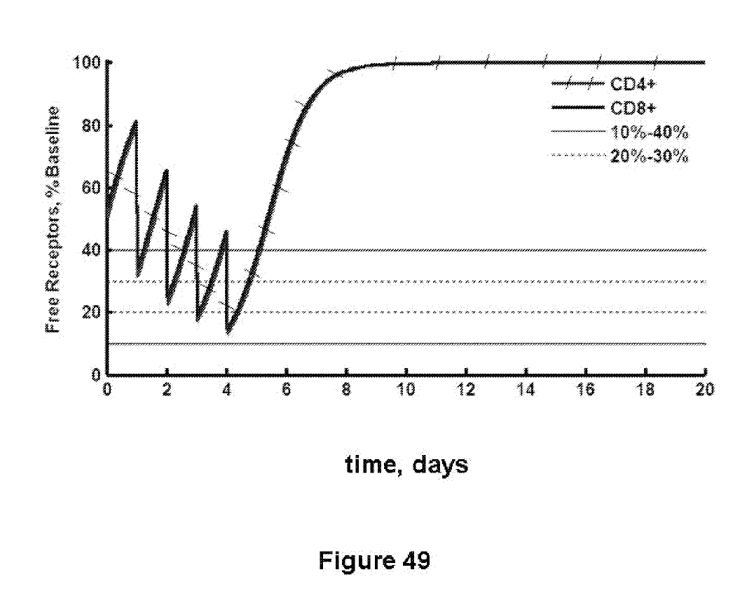

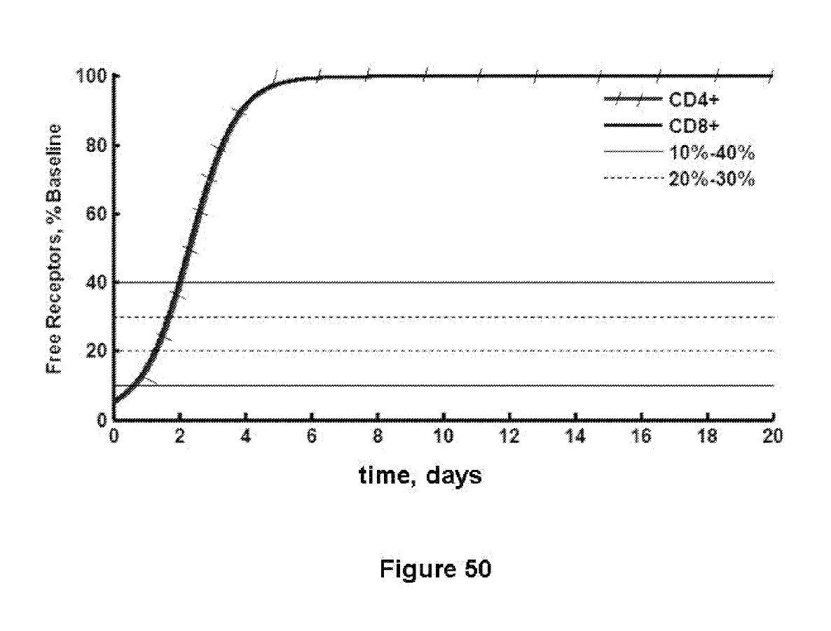

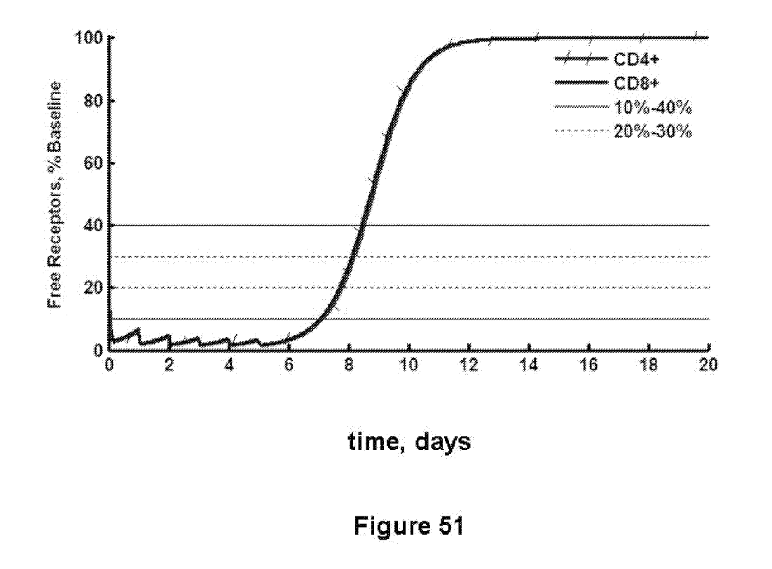

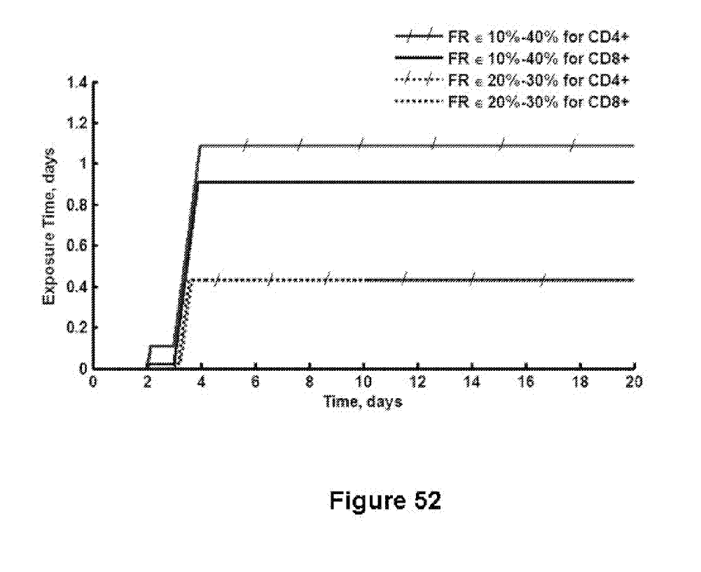

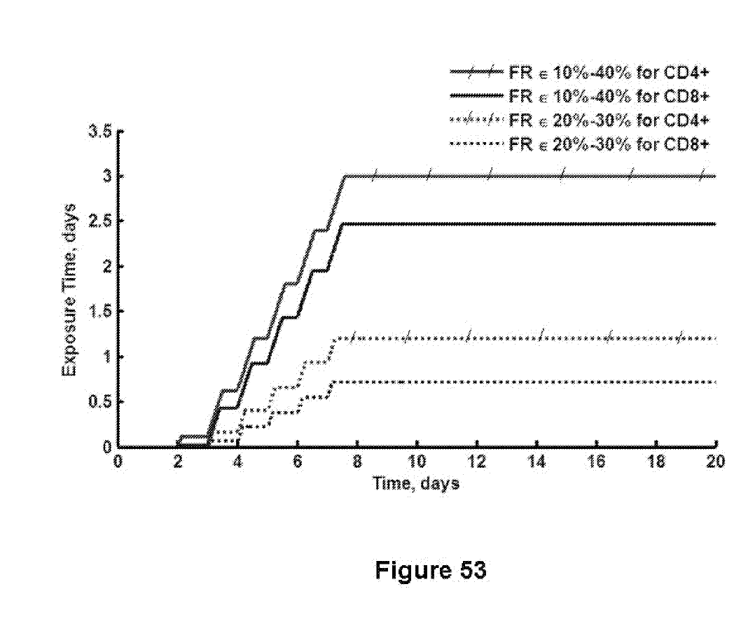

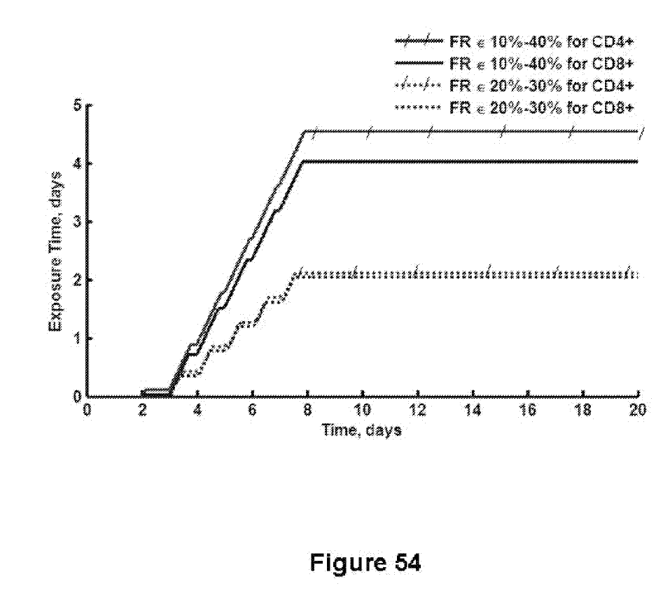

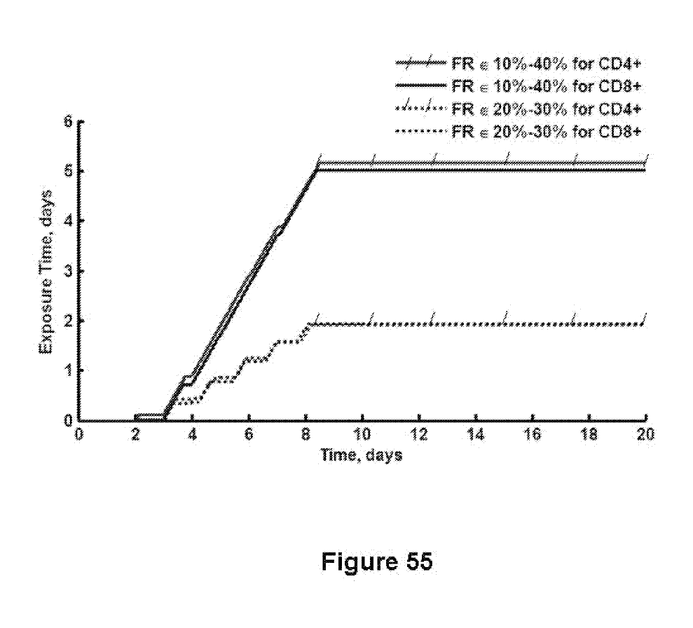

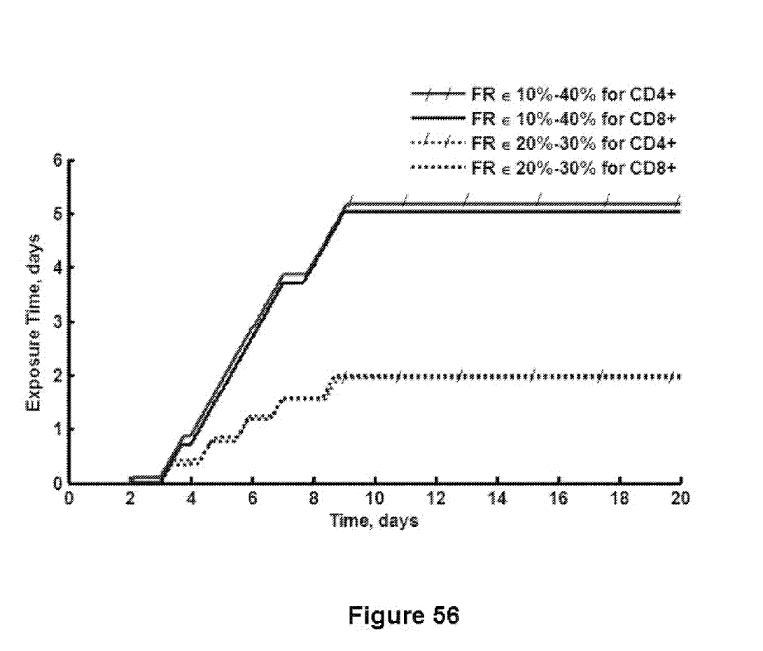

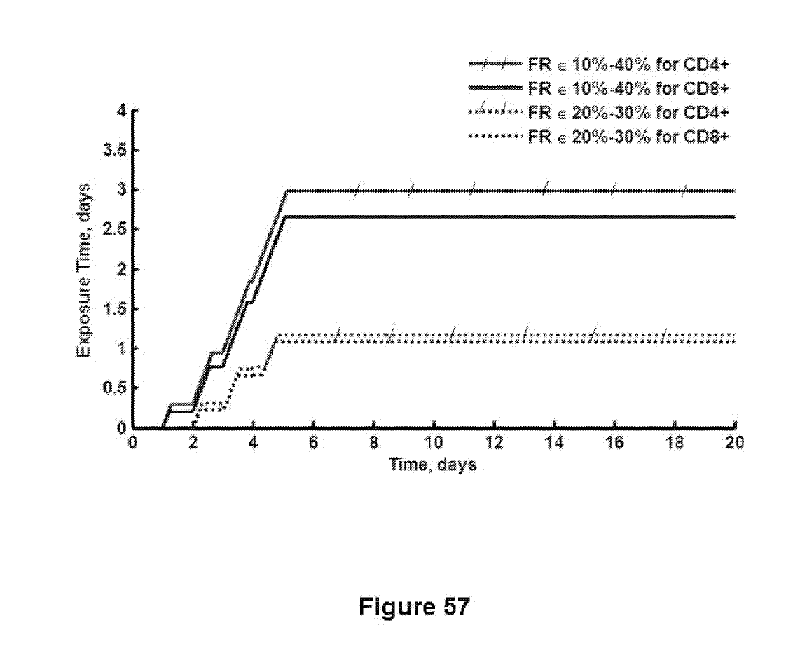

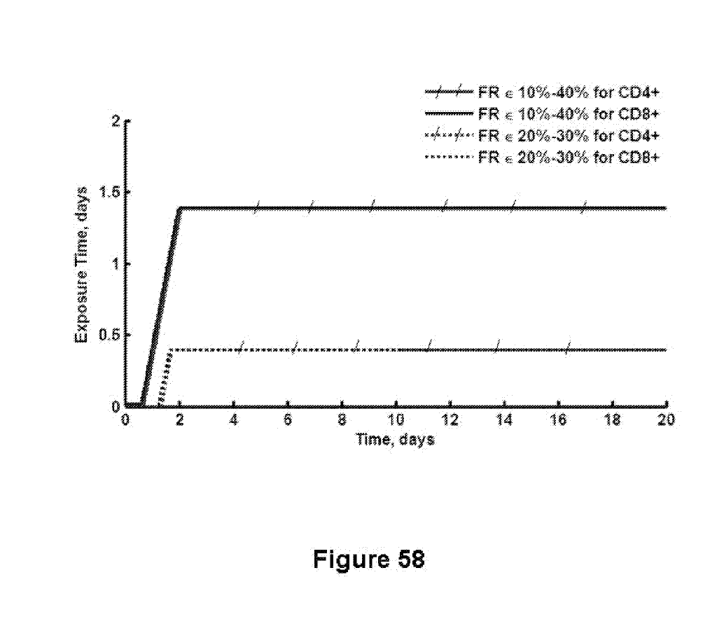

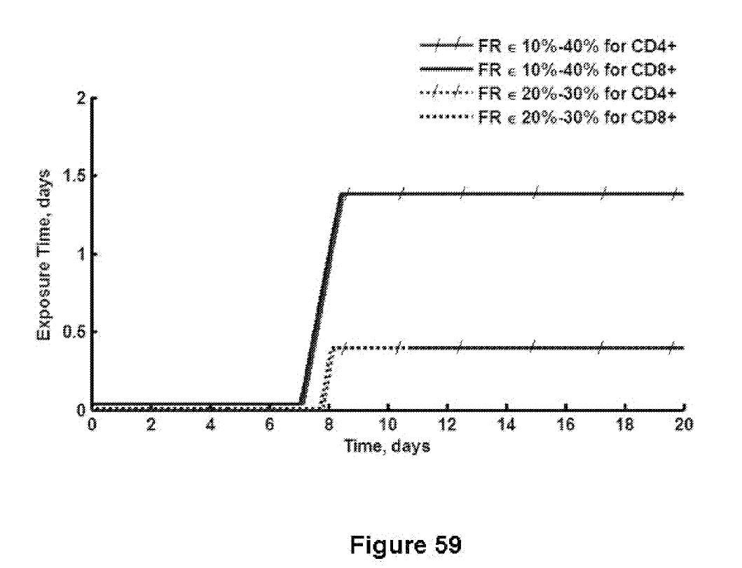

In addition to the data shown in Tables 16 to 23, line graphs were generated showing the level of free receptors on CD4+ and CD8+ T cells after various doses of otelixizumab and indicating the levels of 10%, 20%, 30%, and 40% of baseline values (FIGS. 44 to 51) using the model herein. The data in Tables 24 to 31 and line graphs (FIGS. 52 to 59) were generated showing the time for which T cells (CD4+ and CD8+) (exposure time) expressed levels of 10% to 40% and 20% to 30% of baseline levels after various daily doses of otelixizumab.

TABLE 24
Time in Days for Which CD4+ and CD8+ T cells Had Surface Levels of
Free Receptors (FR) of 10% to 40% and 20% to 30% of Baseline Levels
after Various Daily Doses of Otelixizumab in Cohort C

| | |
|---|---|
| Exposure Time | Exposure Time |
| [days] for | [days] for |
| FR >10% and | FR >20% and |
| FR <40% | FR <30% |

| Dose No. | CD4+ | CD8+ | CD4+ | CD8+ |
|---|---|---|---|---|
| 1 | 0 | 0 | 0 | 0 |
| 2 | 0 | 0 | 0 | 0 |
| 3 | 0.11 | 0.02 | 0 | 0 |
| 4 | 0.98 | 0.89 | 0.43 | 0.43 |
| Total | 1.09 | 0.91 | 0.43 | 0.43 |

TABLE 25
Time in Days for Which CD4+ and CD8+ T cells Had Surface Levels of
10% to 40% and 20% to 30% of Baseline Levels of Free Receptors (FR)
after Various Daily Doses of Otelixizumab in TTEDD CH1

| | |
|---|---|
| Exposure Time | Exposure Time |
| [days] for FR | [days] for FR |
| >10% and | >20% and |
| FR <40% | FR <30% |

| Dose No. | CD4+ | CD8+ | CD4+ | CD8+ |
|---|---|---|---|---|
| 1 | 0 | 0 | 0 | 0 |
| 2 | 0 | 0 | 0 | 0 |
| 3 | 0.11 | 0.02 | 0 | 0 |
| 4 | 0.51 | 0.41 | 0.16 | 0.07 |
| 5 | 0.58 | 0.49 | 0.24 | 0.15 |
| 6 | 0.6 | 0.51 | 0.26 | 0.16 |
| 7 | 0.6 | 0.52 | 0.27 | 0.17 |
| 8 | 0.6 | 0.52 | 0.27 | 0.17 |
| Total | 3 | 2.47 | 1.2 | 0.72 |

TABLE 26
Time in Days for Which CD4+ and CD8+ T cells Had Surface Levels of
10% to 40% and 20% to 30% of Baseline Levels of Free Receptors (FR)
after Various Daily Doses of Otelixizumab in TTEDD CH2

| | |
|---|---|
| Exposure Time | Exposure Time |
| [days] for | [days] for |
| FR >10% and | FR >20% and |
| FR <40% | FR <30% |

| Dose No. | CD4+ | CD8+ | CD4+ | CD8+ |
|---|---|---|---|---|
| 1 | 0 | 0 | 0 | 0 |
| 2 | 0 | 0 | 0 | 0 |
| 3 | 0.11 | 0.02 | 0 | 0 |
| 4 | 0.77 | 0.69 | 0.43 | 0.35 |
| 5 | 0.89 | 0.8 | 0.42 | 0.43 |
| 6 | 0.92 | 0.83 | 0.42 | 0.42 |
| 7 | 0.93 | 0.84 | 0.42 | 0.42 |
| 8 | 0.93 | 0.84 | 0.42 | 0.42 |
| Total | 4.55 | 4.02 | 2.11 | 2.04 |

TABLE 27
Time in Days for Which CD4+ and CD8+ T cells Had Surface Levels of
10% to 40% and 20% to 30% of Baseline Levels of Free Receptors (FR)
after Various Daily Doses of Otelixizumab in TTEDD CH3

| | |
|---|---|
| Exposure Time | Exposure Time |
| [days] for | [days] for |
| FR >10% and | FR >20% and |
| FR <40% | FR <30% |

| Dose No. | CD4+ | CD8+ | CD4+ | CD8+ |
|---|---|---|---|---|
| 1 | 0 | 0 | 0 | 0 |
| 2 | 0 | 0 | 0 | 0 |
| 3 | 0.11 | 0.02 | 0 | 0 |
| 4 | 0.77 | 0.69 | 0.43 | 0.35 |
| 5 | 1 | 0.98 | 0.41 | 0.42 |
| 6 | 1 | 1 | 0.41 | 0.42 |
| 7 | 0.96 | 1 | 0.31 | 0.4 |
| 8 | 1.32 | 1.32 | 0.36 | 0.35 |
| Total | 5.16 | 5.01 | 1.92 | 1.94 |

TABLE 28
Time in Days for Which CD4+ and CD8+ T cells Had Surface Levels of
10% to 40% and 20% to 30% of Baseline Levels of Free Receptors (FR)
after Various Daily Doses of Otelixizumab in TTEDD CH4

| | |
|---|---|
| Exposure Time | Exposure Time |
| [days] for | [days] for |
| FR >10% and | FR >20% and |
| FR <40% | FR <30% |

| Dose No. | CD4+ | CD8+ | CD4+ | CD8+ |
|---|---|---|---|---|
| 1 | 0 | 0 | 0 | 0 |
| 2 | 0 | 0 | 0 | 0 |
| 3 | 0.11 | 0.02 | 0 | 0 |
| 4 | 0.77 | 0.69 | 0.43 | 0.35 |
| 5 | 1 | 0.98 | 0.41 | 0.42 |
| 6 | 1 | 1 | 0.41 | 0.42 |
| 7 | 0.96 | 1 | 0.31 | 0.4 |
| 8 | 1.34 | 1.34 | 0.4 | 0.4 |
| Total | 5.18 | 5.03 | 1.96 | 1.99 |

TABLE 29
Time in Days for Which CD4+ and CD8+ T cells Had Surface Levels of
10% to 40% and 20% to 30% of Baseline Levels of Free Receptors (FR)
after Various Daily Doses of Otelixizumab in TTEDD CH5

| | |
|---|---|
| Exposure Time | Exposure Time |
| [days] for | [days] for |
| FR >10% and | FR >20% and |
| FR <40% | FR <30% |

| Dose No. | CD4+ | CD8+ | CD4+ | CD8+ |
|---|---|---|---|---|
| 1 | 0 | 0 | 0 | 0 |
| 2 | 0.29 | 0.2 | 0 | 0 |
| 3 | 0.65 | 0.56 | 0.31 | 0.23 |
| 4 | 0.9 | 0.82 | 0.43 | 0.43 |
| 5 | 1.15 | 1.07 | 0.43 | 0.42 |
| Total | 2.99 | 2.65 | 1.17 | 1.08 |

TABLE 30
Time in Days for Which CD4+ and CD8+ T cells Had Surface Levels of
10% to 40% and 20% to 30% of Baseline Levels of Free Receptors (FR)
after Various Daily Doses of Otelixizumab in Study II, Cohort 3.

| | |
|---|---|
| Exposure Time | Exposure Time |
| [days] for | [days] for |
| FR >10% and | FR >20% and |
| FR <40% | FR <30% |

| Dose No. | CD4+ | CD8+ | CD4+ | CD8+ |
|---|---|---|---|---|
| 1 | 1.38 | 1.39 | 0.4 | 0.4 |
| Total | 1.38 | 1.39 | 0.4 | 0.4 |

TABLE 31
Time in Days for Which CD4+ and CD8+ T cells Had Surface Levels of
10% to 40% and 20% to 30% of Baseline Levels of Free Receptors (FR)
after Various Daily Doses of Otelixizumab in BDR, Group B.

| | |
|---|---|
| Exposure Time | Exposure Time |
| [days] for FR | [days] for FR |
| >10% and | >20% and |
| FR <40% | FR <30% |

| Dose No. | CD4+ | CD8+ | CD4+ | CD8+ |
|---|---|---|---|---|
| 1 | 0.04 | 0.04 | 0 | 0.01 |
| 2 | 0 | 0 | 0 | 0 |
| 3 | 0 | 0 | 0 | 0 |
| 4 | 0 | 0 | 0 | 0 |
| 5 | 0 | 0 | 0 | 0 |
| 6 | 1.34 | 1.34 | 0.4 | 0.39 |
| Total | 1.38 | 1.38 | 0.4 | 0.4 |

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a human with an anti-CD3 antibody or an antigen binding fragment thereof, the method comprising:
administering the antibody or the fragment to the human in a regimen such that:
(a) in a therapy window of at least 2 days and no more than 6 days, for at least 48 hours of the window, the mean level of free CD3/TCR complexes on CD4+ and on CD8+ T cells is at least 10 percent and less than 40 percent of the mean baseline level;
(b) in a therapy window of 7 days or more, for at least 48 hours of the first 6 days of the window, the mean level of free CD3/TCR complexes on CD4+ and on CD8+ T cells is at least 10 percent and less than 40 percent of the mean baseline level;
(c) in a therapy window of at least 8 days, for at least 48 hours of the window, the mean level of free CD3/TCR complexes on CD4+ and on CD8+ T cells is at least 10 percent and less than 40 percent of the mean baseline level and at least 30 of the 48 hours occur after the first 6 days of the window; or
(d) in a therapy window of at least 4 days, for at least 90 hours of the window, the mean level of free CD3/TCR complexes on CD4+ and on CD8+ T cells is at least 10 percent and less than 40 percent of the mean baseline level,
wherein the antibody or fragment does not bind, or has reduced binding, to at least one class of Fc (gamma) receptor as compared to the OKT3 antibody.

2. The method of claim 1, wherein, for (b), (c), and (d), the regimen, the therapy window, or both the regimen and the therapy window are 14 days or more.

3. The method of claim 1, wherein, for (b), (c) and (d), the regimen, the therapy window, or both the regimen and the therapy window are not more than 14 days.

4. The method of claim 1, wherein the time of the therapy window in which the mean level of free CD3/TCR complexes is at least 10 percent and less than 40 percent of the mean baseline level is continuous.

5. The method of claim 1, wherein the time within the therapy window in which the mean level of free CD3/TCR complexes is at least 10 percent and less than 40 percent of the mean baseline level is not continuous.

6. The method of claim 1, wherein at least one dose of the antibody or fragment administered in an administration is greater than 0.5 mg and the maximum daily dose no greater than 3.0 mg.

7. The method of claim 1, wherein the maximum daily dose of the antibody or the fragment is 1.75 mg or less.

8. A method of treating a human with an anti-CD3 antibody or an antigen binding fragment thereof, the method comprising:
administering the antibody or the fragment to the human in a regimen such that:
(a) in a therapy window of at least two days and no more than 6 days, for at least 12 hours of the window, the mean level of free CD3/TCR complexes on CD4+ and on CD8+ T cells is at least 20 percent and less than 30 percent of the mean baseline level;
(b) in a therapy window of 7 days or more, for at least 18 hours of the first 6 days of the window, the mean level of free CD3/TCR complexes on CD4+ and on CD8+ T cells is at least 20 percent and less than 30 percent of the mean baseline level;
(c) in a therapy window of at least 7 days, for at least 24 hours of the window, the mean level of free CD3/TCR complexes on CD4+ and on CD8+ T cells is at least 20 percent and less than 30 percent of the mean baseline level and at least 15 of the at least 24 hours occur after the first 6 days of the window; or
(d) in a therapy window of at least 7 days, for at least 40 hours of the window, the mean level of free CD3/TCR complexes on CD4+ and on CD8+ T cells is at least 20 percent and less than 30 percent of the mean baseline level and at least half of the at least 40 hours in the window occur after the first 6 days of the window,
wherein the antibody or fragment does not bind, or has reduced binding, to at least one class of Fc (gamma) receptor as compared to the OKT3 antibody.

9. The method of claim 8, wherein, for (b), (c), and (d), the regimen, the therapy window, or both the regimen and the therapy window are 14 days or more.

10. The method of claim 8, wherein, for (b), (c) and (d), the regimen, the therapy window, or both the regimen and the therapy window are not more than 14 days.

11. The method of claim 8, wherein the time within the therapy window in which the mean level of free CD3/TCR complexes is at least 20 percent and less than 30 percent of the mean baseline level is not continuous.

12. The method of claim 1, wherein the first at least four days of the regimen is a dosing ramp.

13. A method of treating a human with an anti-CD3 antibody or an antigen binding fragment thereof, the method comprising:
administering the antibody or the fragment to the human in a regimen such that:
(a) in a regimen of 3 days or more, the daily dose administered is at least 1 mg and no greater than 3 mg in any 24 hour period and on each of at least 3 days of the regimen;
(b) in a regimen of 3 days or more, the daily dose administered is at least 1 mg and no greater than 1.75 mg in any 24 hour period and on each of at least 3 days of the regimen;
(c) in a regimen of 3 days or more, the daily dose administered is at least 14 μg/kg and no greater than 42 μg/kg in any 24 hour period and on each of at least 3 days of the regimen;
(d) in a regimen of 3 days or more, the total dose administered is 2.5 mg to 9 mg and no greater than 3 mg on any single day of the regimen;
(e) in a regimen of 3 days or more, the total dose administered is 2.5 mg to 6.6 mg and no greater than 2.2 mg on any single day of the regimen;
(f) in a regimen of 3 days or more, the total dose administered is 35 μg/kg to 126 μg/kg and no greater than 42 μg/kg on any single day of the regimen;
(g) in a regimen of 3 days or more, the total dose administered is 35 μg/kg to 93 μg/kg and no greater than 31 μg/kg on any single day of the regimen;
(h) in a therapy window of at least three days, where a dose is administered over a period of 24 hours or more, the total dose administered to the human is at least 2.5 mg; or
(i) in a therapy window of at least three days, where a dose is administered over a period of 24 hours or more, the total dose administered to the human is at least 35 μg/kg,
wherein the antibody or fragment does not bind, or has reduced binding, to at least one class of Fc (gamma) receptor as compared to the OKT3 antibody and, optionally, the three days are not continuous.

14. A method of treating a human with an anti-CD3 antibody, or an antigen binding fragment thereof, the method comprising administering the antibody or fragment to the human in a regimen that comprises a dosing ramp of at least four days, wherein the antibody or fragment does not bind or has reduced binding to at least one class of the Fc (gamma) receptor as compared to the OKT3 antibody.

15. The method of claim 14, wherein, for at least days two to four of the ramp, the dosing produces a daily decrease in the mean maximum levels of free CD3/TCR complexes on CD4+ and on CD8+ T cells as compared to the mean baseline levels, wherein the differences between the mean maximum levels on any day of the at least day two to day four of the ramp and the mean maximum levels on the preceding day are not greater than 25 percent of the mean maximum levels on the preceding day.

16. The method of claim 14, wherein the first dose of the ramp produces a decrease in the mean maximum levels of free CD3/TCR complexes on CD4+ and on CD8+ T cells as compared to the mean baseline levels of no greater than 30 percent of the mean maximum levels preceding the first dose of the ramp.

17. The method of claim 14, wherein, for at least days two to four of the ramp, the dosing produces a daily decrease in mean maximum levels of free TCR complex molecules on CD4+ and on CD8+ T cells as compared to the mean baseline levels, wherein the differences between the mean maximum levels on any day of the at least day two to day four of the ramp and the mean maximum levels on the preceding day are at least 5 percent of the mean maximum levels on the preceding day.

18. The method of claim 14, wherein, for at least days two through four of the ramp, the dosing of the ramp produces a daily increase in minimum concentration of the anti-CD3 antibody or the fragment (Cmin) in the peripheral blood, optionally peripheral blood plasma, of the human.

19. The method of claim 14, wherein, for at least days two through four of the ramp, the dosing produces a daily increase in the Cmiₙ in the peripheral blood of the human of no greater than 2.5 times the Cₘin in the peripheral blood on the preceding day, when the concentration of the antibody or fragment in the peripheral blood or peripheral blood plasma of the human is greater than 0.002 mg/L.

20. The method of claim 14, wherein the first dose of the ramp produces a Cₘin in the peripheral blood or peripheral blood plasma of the human of no greater than 0.01 mg/L.

21. The method of claim 14, wherein, for at least days two through four of the ramp, the dosing produces a daily increase in Cₘin in the peripheral blood of the human of at least 10 percent as compared to the Cₘin in the peripheral blood or peripheral blood plasma of the human on the preceding day.

22.-37. (canceled)


SEQUENCE LISTING (10 sequences)

SEQ ID NO: 1 - protein, 449 aa, Artificial Sequence
  HUMANIZED MURINE ANTIBODY HEAVY CHAIN
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 216 aa, Artificial Sequence
  HUMANIZED MURINE ANTIBODY LIGHT CHAIN
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 119 aa, Artificial Sequence [sequence omitted]

SEQ ID NO: 4 - protein, 111 aa, Artificial Sequence
  HUMANIZED MURINE ANTIBODY LIGHT CHAIN VARIABLE REGION
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLR

SEQ ID NO: 5 - protein, 5 aa, Mus Musculus
SFPMA

SEQ ID NO: 6 - protein, 17 aa, Mus Musculus
TISTSGGRTYYRDSVKG

SEQ ID NO: 7 - protein, 10 aa, Mus Musculus
FRQYSGGFDY

SEQ ID NO: 8 - protein, 13 aa, Mus Musculus
TLSSGNIENNYVH

SEQ ID NO: 9 - protein, 7 aa, Mus Musculus
DDDKRPD

SEQ ID NO: 10 - protein, 9 aa, Mus Musculus
HSYVSSFNV